(19) United States
(12) Patent Application Publication
(10) Pub. No.: US 2011/0143408 A1
(43) Pub. Date: Jun. 16, 2011

(54) ZYMOMONAS WITH IMPROVED ARABINOSE UTILIZATION

(75) Inventor: JIANJUN YANG, Hockessin, DE (US)

(73) Assignee: E. I. DU PONT DE NEMOURS AND COMPANY, Wilmington, DE (US)

(21) Appl. No.: 12/796,025

(22) Filed: Jun. 8, 2010

Related U.S. Application Data
(60) Provisional application No. 61/218,164, filed on Jun. 18, 2009.
(60) Provisional application No. 61/218,166, filed on Jun. 18, 2009.

(51) Int. Cl. C12P 7/06; C12N 1/21; C12N 15/74

(52) U.S. Cl. 435/161; 435/252.3; 435/471

(57) ABSTRACT

Several strains of arabinose-utilizing Zymomonas were engineered to express an arabinose-proton symporter which was found to provide the strains with improved ability to utilize arabinose. These strains have improved ethanol production in media containing arabinose, either as the sole carbon source or as one sugar in a mixture of sugars.

### STATEMENT OF GOVERNMENT RIGHTS

This invention was made with United States Government support under Contract No. DE-FC36-07G017056 awarded by the Department of Energy. The U.S. Government has certain rights in this invention.

### FIELD OF THE INVENTION

The invention relates to the fields of microbiology and fermentation. More specifically, engineering of Zymomonas strains to confer improved arabinose utilization, and methods of making ethanol using the strains are described.

### BACKGROUND OF THE INVENTION

Production of ethanol by microorganisms provides an alternative energy source to fossil fuels and is therefore an important area of current research. It is desirable that microorganisms producing ethanol, as well as other useful products, be capable of using xylose and arabinose as carbon sources since these are the predominant pentose sugars in hydrolyzed lignocellulosic materials, which can provide an abundantly available, low cost source of carbon substrate for biocatalysts to use in fermentation.

Zymomonas mobilis and other bacterial ethanologens which do not naturally utilize xylose and arabinose may be genetically engineered for utilization of these sugars. To provide for xylose utilization, strains have been engineered to express genes encoding the following proteins: 1) xylose isomerase, which catalyses the conversion of xylose to xylulose; 2) xylulokinase, which phosphorylates xylulose to form xylulose 5-phosphate; 3) transketolase; and 4) transaldolase (U.S. Pat. No. 5,514,583, U.S. Pat. No. 6,566,107; Zhang et al. (1995) Science 267:240-243). To provide for arabinose utilization, additional genes encoding the following proteins have been introduced: 1) L-arabinose isomerase to convert L-arabinose to L-ribulose, 2) L-ribulokinase to convert L-ribulose to L-ribulose-5-phosphate, and 3) L-ribulose-5-phosphate-4-epimerase to convert L-ribulose-5-phosphate to D-xylulose (U.S. Pat. No. 5,843,760).

Though some strains of Z mobilis have been engineered for arabinose utilization, typically only a low percentage of the arabinose present in a fermentation medium is utilized by these engineered strains. There remains a need to improve arabinose utilization in Zymomonas and other bacterial ethanologens to enhance ethanol production when fermentation is in arabinose containing media.

### SUMMARY OF THE INVENTION

The present invention relates to strains of Zymomonas and Zymobacter that are genetically engineered to have improved ability to use arabinose by introducing a gene for expression of an arabinose-proton symporter, and to production of ethanol using these strains. These strains have improved production of ethanol when grown in media containing arabinose.

Accordingly, the invention provides a recombinant microorganism of the genus Zymomonas or Zymobacter that utilizes arabinose to produce ethanol, said microorganism comprising at least one heterologous gene encoding an arabinose-proton symporter.

In addition, the invention provides a process for generating a recombinant microorganism of the genus Zymomonas or Zymobacter that has increased arabinose utilization comprising:

a) providing a recombinant Zymomonas or Zymobacter strain that utilizes arabinose to produce ethanol under suitable conditions; and

b) introducing at least one gene encoding a heterologous arabinose-proton symporter to the strain of (a).

In another embodiment the invention provides a process for producing ethanol comprising:

a) providing a recombinant Zymomonas or Zymobacter strain that utilizes arabinose to produce ethanol, said strain comprising at least one heterologous gene encoding an arabinose-proton symporter;

b) culturing the strain of (a) in a medium comprising arabinose whereby arabinose is converted by said strain to ethanol.

In another embodiment the invention provides a method for improving arabinose utilization by an arabinose-utilizing microorganism comprising:

(a) providing an arabinose-utilizing microorganism wherein said microorganism is selected from the group consisting of a recombinant Zymomonas or Zymobacter strain that utilizes arabinose to produce ethanol;

(b) introducing into the genome of said microorganism at least one heterologous gene encoding an arabinose-proton symporter wherein said symporter is expressed by said microorganism; and

(c) contacting the microorganism of (b) with a medium comprising arabinose, wherein said microorganism metabolizes said arabinose at an increased rate as compared to said microorganism that is lacking the arabinose-proton symporter.

### BRIEF DESCRIPTION OF THE FIGURES AND SEQUENCE DESCRIPTIONS

The invention can be more fully understood from the following detailed description, the Figures, and the accompanying sequence descriptions that form a part of this application.

FIG. 1 shows a diagram of the ethanol fermentation pathway in Zymomonas engineered for xylose and arabinose utilization, where glf means glucose-facilitated diffusion transporter.

FIG. 2 is a drawing of a plasmid map of pARA205.

FIG. 3 is a drawing of a plasmid map of pARA354.

FIG. 4 shows graphs of growth and metabolite profiles of ZW705 (A), ZW705-ara354 (B), and ZW705-ara354A7 (C) in MRM3A2.5X2.5G5 during a 96-hour time course.

FIG. 5 shows graphs of growth and metabolite profiles of ZW705 (A), ZW705-ara354 (B), and ZW705-ara354A7 (C) in MRM3A2.5X2.5G5 during a 96-hour time course.

FIG. 6 is a drawing of a plasmid map of pARA112.

FIG. 7 is a drawing of a plasmid map of pARA113.

FIG. 8 shows graphs of growth and metabolite profiles of ZW705-ara354A7 (A), ZW705-ara354A7-ara112-2 (B), and ZW705-ara354A7-ara112-3 (C) in MRM3A5 during a 96-hour time course.

FIG. 9 shows graphs of growth and metabolite profiles of ZW705-ara354A7 (A), ZW705-ara354A7-ara112-2 (B), and ZW705-ara354A7-ara112-3 (C) in MRM3A2.5X2.5G5 during a 96-hour time course

FIG. 10 shows graphs of growth and metabolite profiles of ZW705-ara354 (A), ZW705-ara354-ara112-1 (B), and ZW705-ara354-ara112-2 (C) in MRM3A5 during a 96-hour time course.

FIG. 11 shows graphs of growth and metabolite profiles of ZW705-ara354 (A), ZW705-ara354-ara112-1 (B), and ZW705-ara354-ara112-2 (C) in MRM3A2.5X2.5G5 during a 96-hour time course.

FIG. 12 shows graphs of growth and metabolite profiles of ZW801-ara354 (A), ZW801-ara354-ara112-5 (B), and ZW801-ara354-ara112-6 (C) in MRM3A5 during a 96-hour time course.

FIG. 13 shows graphs of growth and metabolite profiles of ZW801-ara354 (A), ZW801-ara354-ara112-5 (B), and ZW801-ara354-ara112-6 (C) in MRM3A2.5X2.5G5 during a 96-hour time course.

The following sequences conform with 37 C.F.R. 1.821-1.825 (“Requirements for Patent Applications Containing Nucleotide Sequences and/or Amino Acid Sequence Disclosures—the Sequence Rules”) and consistent with World Intellectual Property Organization (WIPO) Standard ST.25 (1998) and the sequence listing requirements of the EPO and PCT (Rules 5.2 and 49.5(a-bis), and Section 208 and Annex C of the Administrative Instructions). The symbols and format used for nucleotide and amino acid sequence data comply with the rules set forth in 37 C.F.R. §1.822.

TABLE 1
Protein and coding region SEQ ID NOs for arabinose-proton
symporters encoded by araE

| | SEQ ID NO: | SEQ ID NO: |
|---|---|---|
| Organism | coding region | peptide |
| E. coli | 1 | 2 |
| Shigella flexneri | 3 | 4 |
| Shigella boydii | 5 | 6 |
| Shigella dysenteriae | 7 | 8 |
| Salmonella typhimurium | 9 | 10 |
| Salmonella enterica | 11 | 12 |
| Klebsiella pneumoniae | 13 | 14 |
| Klebsiella oxytoca | 15 | 16 |
| Enterobacter cancerogenus | 17 | 18 |
| Bacillus amyloliquefaciens | 19 | 20 |

SEQ ID NOs:21 and 22 are the amino acid sequence and coding region, respectively, for the araA gene of E. coli.

SEQ ID NOs:23 and 24 are the amino acid sequence and coding region, respectively, for the araB gene of E. coli.

SEQ ID NOs:25 and 26 are the amino acid sequence and coding region, respectively, for the araD gene of E. coli.

SEQ ID NO:27 is the nucleotide sequence of the araB-araA DNA fragment PCR product.

SEQ ID NOs:28 and 29 are the nucleotide sequences of primers for PCR amplification of the araB-araA DNA fragment.

SEQ ID NO:30 is the nucleotide sequence of the araD DNA fragment PCR product, iIncluding RBS and 3′ UTR.

SEQ ID NOs:31 and 32 are the nucleotide sequences of primers for PCR amplification of the araD DNA fragment, Including RBS and 3′ UTR.

SEQ ID NO:33 is the nucleotide sequence of the Pgap promoter of Z. mobilis.

SEQ ID NOs:34 and 35 are the nucleotide sequences of primers for PCR amplification of the Pgap promoter DNA fragment.

SEQ ID NO:36 is the nucleotide sequence of the Pgap promoter

DNA fragment PCR product.

SEQ ID NOs:37 and 38 are the nucleotide sequences of primers for PCR amplification of the spectinomycin resistance cassette.

SEQ ID NOs:39 and 40 are the nucleotide sequences of primers for mutagenesis of Pgap to remove the added NcoI site.

SEQ ID NO:41 is the nucleotide sequence of the pARA205 plasmid. SEQ ID NOs:42 and 43 are the nucleotide sequences of primers for PCR amplification of the LDH-L DNA fragment.

SEQ ID NO:44 is the nucleotide sequence of the LDH-L DNA fragment PCR product.

SEQ ID NOs:45 and 46 are the nucleotide sequences of primers for PCR amplification of the LDH-R DNA fragment.

SEQ ID NO:47 is the nucleotide sequence of the LDH-R DNA fragment PCR product.

SEQ ID NO:48 is the nucleotide sequence of the LoxPw-aadA-LoxPw DNA fragment PCR product.

SEQ ID NO:49 is the nucleotide sequence of the pARA354 plasmid.

SEQ ID NOs:50 and 51 are the nucleotide sequences of primers for PCR amplification to check 5′ integration of P_{gap}-araBAD-aadA.

SEQ ID NOs:52 and 53 are the nucleotide sequences of primers for PCR amplification to check 3′ integration of P_{gap}-araBAD-aadA.

SEQ ID NOs:54 and 55 are the nucleotide sequences of primers for PCR amplification of the araE coding region DNA fragment.

SEQ ID NO:56 is the nucleotide sequence of the araE DNA fragment PCR product.

SEQ ID NOs:57 and 58 are the nucleotide sequences of primers for PCR amplification of the araFGH DNA fragment.

SEQ ID NO:59 is the nucleotide sequence of the araFGH DNA fragment PCR product.

SEQ ID NOs:60 and 61 are the nucleotide sequences of primers for PCR amplification of the Actinoplanes missouriensis P_{gi} DNA fragment.

SEQ ID NO:62 is the nucleotide sequence of the Actinoplanes missouriensis GI promoter in the plasmid used as PCR template.

SEQ ID NO:63 is the nucleotide sequence of the Actinoplanes missouriensis P_{gi} DNA fragment PCR product.

SEQ ID NO:64 is the nucleotide sequence of the chloramphenicol resistance marker.

SEQ ID NO:65 is the nucleotide sequence of the pARA112 plasmid.

SEQ ID NO:66 is the nucleotide sequence of the pARA113 plasmid.

### DETAILED DESCRIPTION

The present invention describes improved arabinose-utilizing recombinant Zymomonas or Zymobacter strains that are further engineered to express an arabinose-proton symporter, and a process for engineering the strains by introducing a gene encoding an arabinose-proton symporter. In other aspects, the present invention describes processes for improving arabinose utilization, and for producing ethanol in media comprising arabinose, using said strains. The arabinose-utilizing strains expressing an arabinose-proton symporter have improved arabinose utilization and are useful for producing ethanol in media comprising arabinose.

Ethanol produced by the present strains with improved arabinose utilization may be used as an alternative energy source to fossil fuels.

The following abbreviations and definitions will be used for the interpretation of the specification and the claims.

As used herein, the terms “comprises,” “comprising,” “includes,” “including,” “has,” “having,” “contains” or “containing,” or any other variation thereof, are intended to cover a non-exclusive inclusion. For example, a composition, a mixture, process, method, article, or apparatus that comprises a list of elements is not necessarily limited to only those elements but may include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

“Gene” refers to a nucleic acid fragment that expresses a specific protein, which may include regulatory sequences preceding (5′ non-coding sequences) and following (3′ non-coding sequences) the coding sequence. “Native gene” or “wild type gene” refers to a gene as found in nature with its own regulatory sequences. “Chimeric gene” refers to any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. “Endogenous gene” refers to a native gene in its natural location in the genome of an organism. A “foreign” gene refers to a gene not normally found in the host organism, but that is introduced into the host organism by gene transfer. Foreign genes can comprise native genes inserted into a non-native organism, or chimeric genes.

The term “araE” refers to a gene or genetic construct that encodes a bacterial arabinose-proton symporter protein which is a low affinity and high capacity arabinose transporter with a Km of 1.25×10⁻⁴ M. Genes encoding the arabinose-proton symporter protein may be isolated from a multiplicity of bacteria and those from enteric bacteria, such as Escherichia, Klebsiella, Salmonella, and Shigella are particularly useful in the present invention.

The term “arabinose utilization” when used in the context of a microorganism refers to the ability of that microorganism to utilize arabinose for the production of products, particularly ethanol.

The term “adapted strain” refers to a microorganism that has been selected for growth on a particular carbon source in order to improve it's ability use that carbon source for the production of products. An “arabinose adapted strain” for example is a strain of microorganism that has been selected for growth on high concentrations of arabinose.

The term “genetic construct” refers to a nucleic acid fragment that encodes for expression of one or more specific proteins. In the genetic construct the gene may be native, chimeric, or foreign in nature. Typically a genetic construct will comprise a “coding sequence”. A “coding sequence” refers to a DNA sequence that codes for a specific amino acid sequence.

“Promoter” or “Initiation control regions” refers to a DNA sequence capable of controlling the expression of a coding sequence or functional RNA. In general, a coding sequence is located 3′ to a promoter sequence. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental conditions. Promoters which cause a gene to be expressed in most cell types at most times are commonly referred to as “constitutive promoters”.

The term “expression”, as used herein, refers to the transcription and stable accumulation of sense (mRNA) or antisense RNA derived from a gene. Expression may also refer to translation of mRNA into a polypeptide. “Antisense inhibition” refers to the production of antisense RNA transcripts capable of suppressing the expression of the target protein. “Overexpression” refers to the production of a gene product in transgenic organisms that exceeds levels of production in normal or non-transformed organisms. “Co-suppression” refers to the production of sense RNA transcripts or fragments capable of suppressing the expression of identical or substantially similar foreign or endogenous genes (U.S. Pat. No. 5,231,020).

The term “transformation” as used herein, refers to the transfer of a nucleic acid fragment into a host organism, resulting in genetically stable inheritance. The transferred nucleic acid may be in the form of a plasmid maintained in the host cell, or some transferred nucleic acid may be integrated into the genome of the host cell. Host organisms containing the transformed nucleic acid fragments are referred to as “transgenic” or “recombinant” or “transformed” organisms.

The terms “plasmid” and “vector” as used herein, refer to an extra chromosomal element often carrying genes which are not part of the central metabolism of the cell, and usually in the form of circular double-stranded DNA molecules. Such elements may be autonomously replicating sequences, genome integrating sequences, phage or nucleotide sequences, linear or circular, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell.

The term “operably linked” refers to the association of nucleic acid sequences on a single nucleic acid fragment so that the function of one is affected by the other. For example, a promoter is operably linked with a coding sequence when it is capable of affecting the expression of that coding sequence (i.e., that the coding sequence is under the transcriptional control of the promoter). Coding sequences can be operably linked to regulatory sequences in sense or antisense orientation.

The term “selectable marker” means an identifying factor, usually an antibiotic or chemical resistance gene, that is able to be selected for based upon the marker gene's effect, i.e., resistance to an antibiotic, wherein the effect is used to track the inheritance of a nucleic acid of interest and/or to identify a cell or organism that has inherited the nucleic acid of interest.

As used herein the term “codon degeneracy” refers to the nature in the genetic code permitting variation of the nucleotide sequence without affecting the amino acid sequence of an encoded polypeptide. The skilled artisan is well aware of the “codon-bias” exhibited by a specific host cell in usage of nucleotide codons to specify a given amino acid. Therefore, when synthesizing a gene for improved expression in a host cell, it is desirable to design the gene such that its frequency of codon usage approaches the frequency of preferred codon usage of the host cell.

The term “codon-optimized” as it refers to genes or coding regions of nucleic acid molecules for transformation of various hosts, refers to the alteration of codons in the gene or coding regions of the nucleic acid molecules to reflect the typical codon usage of the host organism without altering the polypeptide encoded by the DNA.

The term “carbon source” refers to sugars such as oligosaccharides and monosaccharides that can be used by a microorganism in a fermentation process (“fermentable sugar”) to produce a product suh as ethanol. A microorganism may have the ability to use a single carbon source for the production of a product and as such the carbon source is refereed to herein as a “sole” carbon source.

The term “lignocellulosic” refers to a composition comprising both lignin and cellulose. Lignocellulosic material may also comprise hemicellulose.

The term “cellulosic” refers to a composition comprising cellulose and additional components, including hemicellulose.

The term “saccharification” refers to the production of fermentable sugars or carbon sources from polysaccharides.

The term “pretreated biomass” means biomass that has been subjected to pretreatment prior to saccharification.

“Biomass” refers to any cellulosic or lignocellulosic material and includes materials comprising cellulose, and optionally further comprising hemicellulose, lignin, starch, oligosaccharides and/or monosaccharides. Biomass may also comprise additional components, such as protein and/or lipid. Biomass may be derived from a single source, or biomass can comprise a mixture derived from more than one source; for example, biomass could comprise a mixture of corn cobs and corn stover, or a mixture of grass and leaves. Biomass includes, but is not limited to, bioenergy crops, agricultural residues, municipal solid waste, industrial solid waste, sludge from paper manufacture, yard waste, wood and forestry waste. Examples of biomass include, but are not limited to, corn cobs, crop residues such as corn husks, corn stover, grasses, wheat, wheat straw, barley straw, hay, rice straw, switchgrass, waste paper, sugar cane bagasse, sorghum bagasse or stover, soybean stover, components obtained from milling of grains, trees, branches, roots, leaves, wood chips, sawdust, shrubs and bushes, vegetables, fruits, flowers and animal manure.

“Biomass hydrolysate” refers to the product resulting from saccharification of biomass. The biomass may also be pretreated or pre-processed prior to saccharification.

The term “heterologous” means not naturally found in the location of interest. For example, a heterologous gene refers to a gene that is not naturally found in the host organism, but that is introduced into the host organism by gene transfer. For example, a heterologous nucleic acid molecule that is present in a chimeric gene is a nucleic acid molecule that is not naturally found associated with the other segments of the chimeric gene, such as the nucleic acid molecules having the coding region and promoter segments not naturally being associated with each other.

As used herein, an “isolated nucleic acid molecule” is a polymer of RNA or DNA that is single- or double-stranded, optionally containing synthetic, non-natural or altered nucleotide bases. An isolated nucleic acid molecule in the form of a polymer of DNA may be comprised of one or more segments of cDNA, genomic DNA or synthetic DNA.

A nucleic acid fragment is “hybridizable” to another nucleic acid fragment, such as a cDNA, genomic DNA, or RNA molecule, when a single-stranded form of the nucleic acid fragment can anneal to the other nucleic acid fragment under the appropriate conditions of temperature and solution ionic strength. Hybridization and washing conditions are well known and exemplified in Sambrook, J., Fritsch, E. F. and Maniatis, T. Molecular Cloning: A Laboratory Manual, 2^{nd} ed., Cold Spring Harbor Laboratory: Cold Spring Harbor, N.Y. (1989), particularly Chapter 11 and Table 11.1 therein (entirely incorporated herein by reference). The conditions of temperature and ionic strength determine the “stringency” of the hybridization. Stringency conditions can be adjusted to screen for moderately similar fragments (such as homologous sequences from distantly related organisms), to highly similar fragments (such as genes that duplicate functional enzymes from closely related organisms). Post-hybridization washes determine stringency conditions. One set of preferred conditions uses a series of washes starting with 6×SSC, 0.5% SDS at room temperature for 15 min, then repeated with 2×SSC, 0.5% SDS at 45° C. for 30 min, and then repeated twice with 0.2×SSC, 0.5% SDS at 50° C. for 30 min. A more preferred set of stringent conditions uses higher temperatures in which the washes are identical to those above except for the temperature of the final two 30 min washes in 0.2×SSC, 0.5% SDS was increased to 60° C. Another preferred set of highly stringent conditions uses two final washes in 0.1×SSC, 0.1% SDS at 65° C. An additional set of stringent conditions include hybridization at 0.1×SSC, 0.1% SDS, 65° C. and washes with 2×SSC, 0.1% SDS followed by 0.1×SSC, 0.1% SDS, for example.

Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the greater the value of Tm for hybrids of nucleic acids having those sequences. The relative stability (corresponding to higher Tm) of nucleic acid hybridizations decreases in the following order: RNA:RNA, DNA:RNA, DNA:DNA. For hybrids of greater than 100 nucleotides in length, equations for calculating Tm have been derived (see Sambrook et al., supra, 9.50-9.51). For hybridizations with shorter nucleic acids, i.e., oligonucleotides, the position of mismatches becomes more important, and the length of the oligonucleotide determines its specificity (see Sambrook et al., supra, 11.7-11.8). In one embodiment the length for a hybridizable nucleic acid is at least about 10 nucleotides. Preferably a minimum length for a hybridizable nucleic acid is at least about 15 nucleotides; more preferably at least about 20 nucleotides; and most preferably the length is at least about 30 nucleotides. Furthermore, the skilled artisan will recognize that the temperature and wash solution salt concentration may be adjusted as necessary according to factors such as length of the probe.

A “substantial portion” of an amino acid or nucleotide sequence is that portion comprising enough of the amino acid sequence of a polypeptide or the nucleotide sequence of a gene to putatively identify that polypeptide or gene, either by manual evaluation of the sequence by one skilled in the art, or by computer-automated sequence comparison and identification using algorithms such as BLAST (Altschul, S. F., et al., J. Mol. Biol., 215:403-410 (1993)). In general, a sequence of ten or more contiguous amino acids or thirty or more nucleotides is necessary in order to putatively identify a polypeptide or nucleic acid sequence as homologous to a known protein or gene. Moreover, with respect to nucleotide sequences, gene specific oligonucleotide probes comprising 20-30 contiguous nucleotides may be used in sequence-dependent methods of gene identification (e.g., Southern hybridization) and isolation (e.g., in situ hybridization of bacterial colonies or bacteriophage plaques). In addition, short oligonucleotides of 12-15 bases may be used as amplification primers in PCR in order to obtain a particular nucleic acid fragment comprising the primers. Accordingly, a “substantial portion” of a nucleotide sequence comprises enough of the sequence to specifically identify and/or isolate a nucleic acid fragment comprising the sequence. The instant specification teaches the complete amino acid and nucleotide sequence encoding particular fungal proteins. The skilled artisan, having the benefit of the sequences as reported herein, may now use all or a substantial portion of the disclosed sequences for purposes known to those skilled in this art. Accordingly, the instant invention comprises the complete sequences as reported in the accompanying Sequence Listing, as well as substantial portions of those sequences as defined above.

The term “complementary” is used to describe the relationship between nucleotide bases that are capable of hybridizing to one another. For example, with respect to DNA, adenosine is complementary to thymine and cytosine is complementary to guanine.

The terms “homology” and “homologous” are used interchangeably herein. They refer to nucleic acid fragments wherein changes in one or more nucleotide bases do not affect the ability of the nucleic acid fragment to mediate gene expression or produce a certain phenotype. These terms also refer to modifications of the nucleic acid fragments of the instant invention such as deletion or insertion of one or more nucleotides that do not substantially alter the functional properties of the resulting nucleic acid fragment relative to the initial, unmodified fragment. It is therefore understood, as those skilled in the art will appreciate, that the invention encompasses more than the specific exemplary sequences.

Moreover, the skilled artisan recognizes that homologous nucleic acid sequences encompassed by this invention are also defined by their ability to hybridize, under moderately stringent conditions (e.g., 0.5×SSC, 0.1% SDS, 60° C.) with the sequences exemplified herein, or to any portion of the nucleotide sequences disclosed herein and which are functionally equivalent to any of the nucleic acid sequences disclosed herein.

The term “percent identity”, as known in the art, is a relationship between two or more polypeptide sequences or two or more polynucleotide sequences, as determined by comparing the sequences. In the art, “identity” also means the degree of sequence relatedness between polypeptide or polynucleotide sequences, as the case may be, as determined by the match between strings of such sequences. “Identity” and “similarity” can be readily calculated by known methods, including but not limited to those described in: 1.) Computational Molecular Biology (Lesk, A. M., Ed.) Oxford University: NY (1988); 2.) Biocomputing: Informatics and Genome Projects (Smith, D. W., Ed.) Academic: NY (1993); 3.) Computer Analysis of Sequence Data, Part I (Griffin, A. M., and Griffin, H. G., Eds.) Humania: NJ (1994); 4.) Sequence Analysis in Molecular Biology (von Heinje, G., Ed.) Academic (1987); and 5.) Sequence Analysis Primer (Gribskov, M. and Devereux, J., Eds.) Stockton: NY (1991).

Preferred methods to determine identity are designed to give the best match between the sequences tested. Methods to determine identity and similarity are codified in publicly available computer programs. Sequence alignments and percent identity calculations may be performed using the MegAlign™ program of the LASERGENE bioinformatics computing suite (DNASTAR Inc., Madison, Wis.). Multiple alignment of the sequences is performed using the “Clustal method of alignment” which encompasses several varieties of the algorithm including the “Clustal V method of alignment” corresponding to the alignment method labeled Clustal V (described by Higgins and Sharp, CABIOS. 5:151-153 (1989); Higgins, D. G. et al., Comput. Appl. Biosci., 8:189-191 (1992)) and found in the MegAlign™ program of the LASERGENE bioinformatics computing suite (DNASTAR Inc.). For multiple alignments, the default values correspond to GAP PENALTY=10 and GAP LENGTH PENALTY=10. Default parameters for pairwise alignments and calculation of percent identity of protein sequences using the Clustal method are KTUPLE=1, GAP PENALTY=3, WINDOW=5 and DIAGONALS SAVED=5. For nucleic acids these parameters are KTUPLE=2, GAP PENALTY=5, WINDOW=4 and DIAGONALS SAVED=4. After alignment of the sequences using the Clustal V program, it is possible to obtain a “percent identity” by viewing the “sequence distances” table in the same program. Additionally the “Clustal W method of alignment” is available and corresponds to the alignment method labeled Clustal W (described by Higgins and Sharp, CABIOS. 5:151-153 (1989); Higgins, D. G. et al., Comput. Appl. Biosci. 8:189-191 (1992)) and found in the MegAlign™ v6.1 program of the LASERGENE bioinformatics computing suite (DNASTAR Inc.). Default parameters for multiple alignment (GAP PENALTY=10, GAP LENGTH PENALTY=0.2, Delay Divergen Seqs(%)=30, DNA Transition Weight=0.5, Protein Weight Matrix=Gonnet Series, DNA Weight Matrix=IUB). After alignment of the sequences using the Clustal W program, it is possible to obtain a “percent identity” by viewing the “sequence distances” table in the same program.

It is well understood by one skilled in the art that many levels of sequence identity are useful in identifying polypeptides, from other species, wherein such polypeptides have the same or similar function or activity. Useful examples of percent identities include, but are not limited to: 24%, 30%, 35%, 40%, 45%, 50%, 55%, 60%, 65%, 70%, 75%, 80%, 85%, 90%, or 95%, or any integer percentage from 24% to 100% may be useful in describing the present invention, such as 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%. Suitable nucleic acid fragments not only have the above homologies but typically encode a polypeptide having at least 50 amino acids, preferably at least 100 amino acids, more preferably at least 150 amino acids, still more preferably at least 200 amino acids, and most preferably at least 250 amino acids.

The term “sequence analysis software” refers to any computer algorithm or software program that is useful for the analysis of nucleotide or amino acid sequences. “Sequence analysis software” may be commercially available or independently developed. Typical sequence analysis software will include, but is not limited to: 1.) the GCG suite of programs (Wisconsin Package Version 9.0, Genetics Computer Group (GCG), Madison, Wis.); 2.) BLASTP, BLASTN, BLASTX (Altschul et al., J. Mol. Biol., 215:403-410 (1990)); 3.) DNASTAR (DNASTAR, Inc. Madison, Wis.); 4.) Sequencher (Gene Codes Corporation, Ann Arbor, Mich.); and 5.) the FASTA program incorporating the Smith-Waterman algorithm (W. R. Pearson, Comput. Methods Genome Res., [Proc. Int. Symp.] (1994), Meeting Date 1992, 111-20. Editor(s): Suhai, Sandor. Plenum: New York, N.Y.). Within the context of this application it will be understood that where sequence analysis software is used for analysis, that the results of the analysis will be based on the “default values” of the program referenced, unless otherwise specified. As used herein “default values” will mean any set of values or parameters that originally load with the software when first initialized.

Standard recombinant DNA and molecular cloning techniques used here are well known in the art and are described by Sambrook, J., Fritsch, E. F. and Maniatis, T. Molecular Cloning: A Laboratory Manual, 2^{nd} ed.; Cold Spring Harbor Laboratory: Cold Spring Harbor, N.Y., 1989 (hereinafter “Maniatis”); and by Silhavy, T. J., Bennan, M. L. and Enquist, L. W. Experiments with Gene Fusions; Cold Spring Harbor Laboratory: Cold Spring Harbor, N.Y., 1984; and by Ausubel, F. M. et al., In Current Protocols in Molecular Biology, published by Greene Publishing and Wiley-Interscience, 1987.

The present invention relates to engineered strains of arabinose-utilizing Zymomonas or Zymobacter that have improved arabinose utilization when fermented in arabinose containing media, and to processes for ethanol production using the strains. A challenge for improving ethanol production by fermentation of a biocatalyst in media that includes biomass hydrolysate, produced typically by pretreatment and saccharification of biomass, is obtaining efficient utilization of arabinose. Arabinose is one of the predominant pentose sugars in hydrolyzed lignocellulosic materials, the other being xylose. Applicants have discovered that expression of an arabinose-proton symporter leads to increased efficiency in arabinose utilization by arabinose-utilizing strains, and thus to higher ethanol yields when fermentation is in arabinose containing media.

### Arabinose-Utilizing Host Strain

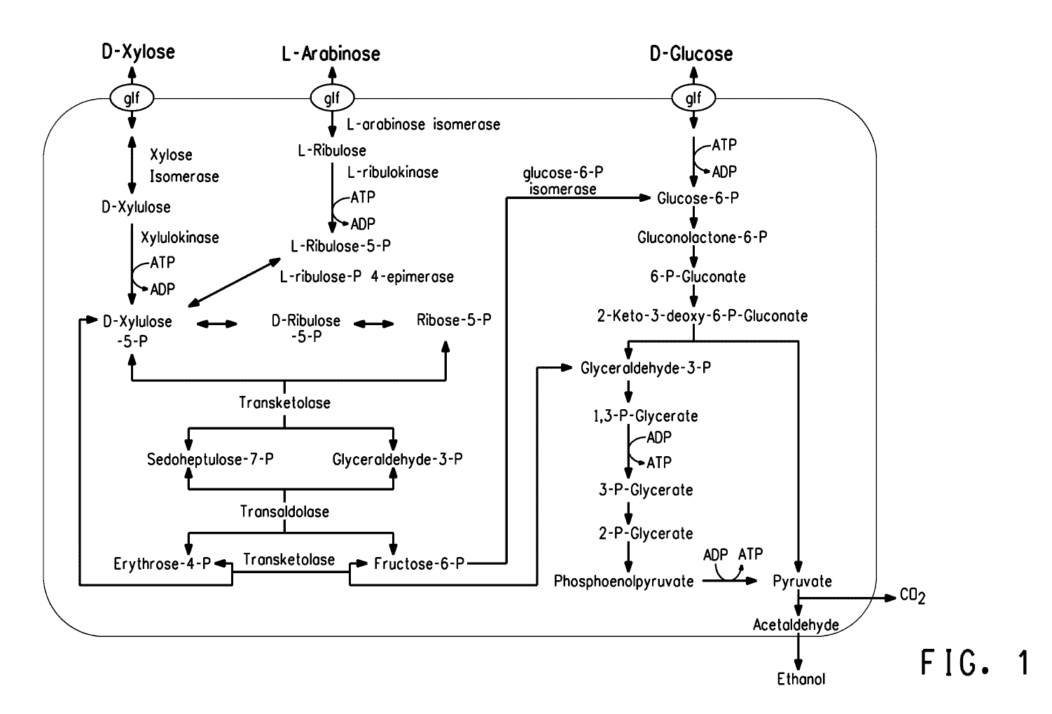

Any strain of Zymomonas or Zymobacter that is able to utilize arabinose as a carbon source may be used as a host for preparing the strains of the present invention. Strains of Zymomonas, such as Z. mobilis that have been engineered for arabinose fermentation to ethanol are particularly useful. Zymomonas has been engineered for arabinose utilization by introducing genes encoding 1) L-arabinose isomerase to convert L-arabinose to L-ribulose, 2) L-ribulokinase to convert L-ribulose to L-ribulose-5-phosphate, and 3) L-ribulose-5-phosphate-4-epimerase to convert L-ribulose-5-phosphate to D-xylulose (U.S. Pat. No. 5,843,760 and described in Examples 1 and 2 herein; see diagram in FIG. 1). DNA sequences encoding these enzymes may be obtained from any microorganisms that are able to metabolize arabinose. Sources for the coding regions include Klebsiella, Escherichia, Rhizobium, Agrobacterium, and Salmonella. Particularly useful are the coding regions of E. coli which are for L-arabinose isomerase: coding region of araA (coding region SEQ ID NO:21; protein SEQ ID NO:22), for L-ribulokinase: coding region of araB (coding region SEQ ID NO:23; protein SEQ ID NO:24), and for L-ribulose-5-phosphate-4-epimerase: coding region of araD (coding region SEQ ID NO:25; protein SEQ ID NO:26). These proteins and their coding regions may be readily identified in other arabinose utilizing microorganisms, such as those listed above, by one skilled in the art using bioinformatics or experimental methods as described below for araE.

In addition, transketolase and transaldolase activities are used in the biosynthetic pathway from arabinose to ethanol (see FIG. 1). Transketolase and transaldolase are two enzymes of the pentose phosphate pathway that convert xylulose 5-phosphate to intermediates that couple pentose metabolism to the glycolytic Entner-Douderoff pathway permitting the metabolism of arabinose or xylose to ethanol. These may be endogenous activities, or endogenous activities may complement introduced activities for these enzymes.

Typically, arabinose-utilizing Zymomonas is also engineered for xylose utilization. Typically four genes have been introduced into Z mobilis for expression of four enzymes involved in xylose metabolism (FIG. 1) as described in U.S. Pat. No. 5,514,583, which is herein incorporated by reference. These include genes encoding transketolase and transaldolase as described above, as well as xylose isomerase, which catalyzes the conversion of xylose to xylulose and xylulokinase, which phosphorylates xylulose to form xylulose 5-phosphate (see FIG. 1). DNA sequences encoding these enzymes may be obtained from any of numerous microorganisms that are able to metabolize xylose, such as enteric bacteria, and some yeasts and fungi. Sources for the coding regions include Xanthomonas, Klebsiella, Escherichia, Rhodobacter, Flavobacterium, Acetobacter, Gluconobacter, Rhizobium, Agrobacterium, Salmonella, Pseudomonads, and Zymomonas. Particularly useful are the coding regions of E. coli.

For expression, the encoding DNA sequences for arabinose-utilizing proteins and xylose-utilizing proteins are operably linked to promoters that are expressed in Z. mobilis cells, and transcription terminators. Examples of promoters that may be used include the promoters of the Z. mobilis glyceraldehyde-3-phosphate dehydrogenase encoding gene (GAP promoter; Pgap), of the Z. mobilis enolase encoding gene (ENO promoter; Peno), and of the Actinoplanes missouriensis xylose isomerase encoding gene (GI promoter, Pgi). The coding regions may be individually expressed from a promoter typically as a chimeric gene, or two or more coding regions may be joined in an operon with expression from the same promoter. The resulting chimeric genes and/or operons are typically constructed in or transferred to a vector for further manipulations.

Vectors are well known in the art. Particularly useful for expression in Zymomonas are vectors that can replicate in both E. coli and Zymomonas, such as pZB188 which is described in U.S. Pat. No. 5,514,583. Vectors may include plasmids for autonomous replication in a cell, and plasmids for carrying constructs to be integrated into the cell genome. Plasmids for DNA integration may include transposons, regions of nucleic acid sequence homologous to the target cell genome, site-directed integration sequences, or other sequences supporting integration. In homologous recombination, DNA sequences flanking a target integration site are placed bounding a spectinomycin-resistance gene, or other selectable marker, and the desired chimeric gene leading to insertion of the selectable marker and chimeric gene into the target genomic site as described in Example 2 herein. In addition, the selectable marker may be bounded by site-specific recombination sites, so that after expression of the corresponding site-specific recombinase, the resistance gene may be excised from the genome.

Xylose-utilizing strains that are of particular use include CP4(pZB5) (U.S. Pat. No. 5,514,583), ATCC31821/pZB5 (U.S. Pat. No. 6,566,107), 8b (US 20030162271; Mohagheghi et al., (2004) Biotechnol. Lett. 25; 321-325), and ZW658 with derivatives ZW800 and ZW801-4 (commonly owned and co-pending US Patent App. Pub. #US20080286870; deposited, ATTCC # PTA-7858). Also ZW705 may be used, which is described in commonly owned and co-pending U.S. patent application Ser. No. 12/641,642, which is herein incorporated by reference. Arabinose utilizing strains that may be used are disclosed in U.S. Pat. No. 5,843,760, which is herein incorporated by reference, as well as being described herein in Examples 1 and 2.

### Adaptation for Arabinose Utilization

A Z. mobilis strain engineered for xylose and arabinose utilization as described above was found by Applicants to utilize about 33% of arabinose in media where arabinose is the sole carbon source (at 50 g/L), and about 68% of arabinose in media including mixed sugars of 25 g/L arabinose, 25 g/L xylose, and 50 g/L glucose in test growth conditions. In an attempt to derive a strain with improved arabinose utilization, applicants adapted cells from the xylose and arabinose utilizing strain by serial growth in media with 50 g/L arabinose as the sole carbon source as described herein in Example 2. Using this process, isolated strains were obtained that had a substantial improvement in arabinose utilization in media where arabinose is the sole carbon source, which are arabinose-adapted strains. For example, one strain used about 83% of arabinose in media where 50 g/L arabinose is the sole carbon source. In mixed sugars media containing 25 g/L arabinose, 25 g/L xylose, and 50 g/L glucose, there was less improvement: about 74% of arabinose was used. Also in mixed sugars media arabinose utilization was delayed as compared to utilization of glucose and xylose.

To obtain strains with improved arabinose utilization, strains engineered for expression of arabinose utilization genes as described above may be adapted by serial growth in media containing arabinose as the sole carbon source in concentrations between about 20 g/L and 100 g/L, or higher. Adaptation may be in lower concentrations of arabinose, but with initial growth in about 20 g/L or higher. Serial growth is typically for at least about 25 doublings. Adaptation may be before or after introducing a heterologous arabinose-proton symporter, that is described below, to an arabinose utilizing strain. In addition, cells may be adapted both before and after introduction of a heterologous arabinose-proton symporter.

### Discovery for Engineering Improved Arabinose Utilization

Applicants engineered xylose and arabinose utilizing strains of Zymomonas for expression of the two different arabinose transport systems present in E. coli. The two systems are 1) an ABC transporter consisting of three proteins encoded by araFGH: 33 kD preiplasmic arabinose binding protein encoded by araF, 55 kD membrane bound ATPase encoded by araG, and 34 kD membrane bound protein encoded by araH; and 2) an arabinose-proton symporter consisting of one protein: 52 kD arabinose-proton symporter encoded by araE. The ABC transporter is a high affinity and low capacity arabinose transporter with a Km of 3×10⁻⁶ M, while the arabinose-proton symporter is a low affinity and high capacity arabinose transporter with a Km of 1.25×10⁻⁴ M. Applicants found that expression of the ABC transporter actually resulted in reduced arabinose utilization in arabinose only media. Expression of the arabinose-proton symporter increased arabinose utilization in both arabinose only media and mixed sugars media. Thus applicants have discovered that the E. coli ABC transporter does not improve arabinose utilization while the arabinose-proton symporter does improve arabinose utilization in Zymomonas. With expression of the arabinose-proton symporter, arabinose utilization was greatly increased in both arabinose only media and in mixed sugars media.

Expression of an arabinose-proton symporter increased arabinose utilization in all strains tested. These include an arabinose and xylose utilizing Z. mobilis strain with no adaptation, an arabinose and xylose utilizing Z. mobilis strain that had been adapted for xylose utilization in stress conditions (disclosed in commonly owned and co-pending U.S. patent application Ser. No. 12/641,642, which is herein incorporated by reference), and an arabinose and xylose utilizing Z. mobilis strain that had been adapted for xylose utilization in stress conditions and also for arabinose utilization as described herein above and in Example 2. In strains without arabinose adaptation, arabinose utilization was increased by at least about 28% in arabinose only media as well as in mixed sugars media. Also in an arabinose adapted strain, arabinose utilization was increased by at least about 28% in mixed sugars media. In arabinose only media the level of arabinose utilization in the arabinose adapted parental strain without expression of the arabinose-proton symporter is already at about 80%, and therefore the increase in arabinose utilization cannot exceed 20%, and is about 18%.

Thus any Zymomonas or Zymobacter strain that is capable of utilizing arabinose, also called an arabinose utilizing strain, may be used to create the present strains. Particularly useful are strains that additionally utilize xylose and glucose. In these strains arabinose utilization is improved by at least about 10% by expressing an arabinose-proton symporter. Arabinose utilization may be improved by at least about 10%, 12%, 16%, 18%, 20%, 24%, 28%, or more. The % improvement may vary depending on the growth conditions used including the type of media and the parental microorganism used for engineering expression of the arabinose-proton symporter, as well as the specific resulting engineered strain. Factors causing variation include level of expression of the introduced arabinose-proton symporter and resulting transporter activity level, which may vary between transformants.

### Expression of an Arabinose-Proton Symporter

In the present engineered Zymomonas or Zymobacter cells any bacterial arabinose-proton symporter may be expressed to provide increased arabinose utilization. Bacterial arabinose-proton symporter proteins and their encoding sequences for expression in Zymomonas or Zymobacter are heterologous, as they are not naturally found in Zymomonas or Zymobacter. Examples of arabinose-proton symporter protein and encoding sequences that may be expressed include those encoded by the araE genes of E. coli (coding region SEQ ID NO:1; protein SEQ ID NO:2), Shigella flexneri (coding region SEQ ID NO:3; protein SEQ ID NO:4), Shigella boydii (coding region SEQ ID NO:5; protein SEQ ID NO:6), Shigella dysenteriae (coding region SEQ ID NO:7; protein SEQ ID NO:8), Salmonella typhimurium (coding region SEQ ID NO:9; protein SEQ ID NO:10), Salmonella enterica (coding region SEQ ID NO:11; protein SEQ ID NO:12), Klebsiella pneumoniae (coding region SEQ ID NO13; protein SEQ ID NO:14), Klebsiella oxytoca (coding region SEQ ID NO:15; protein SEQ ID NO:16), Enterobacter cancerogenus (coding region SEQ ID NO:17; protein SEQ ID NO:18) and Bacillus amyloliquefaciens (coding region SEQ ID NO:19; protein SEQ ID NO:20).

Because the sequences of arabinose-proton symporter coding regions and the encoded proteins are well known, as exemplified in the SEQ ID NOs listed above and given in Table 1, additional suitable arabinose-proton symporters may be readily identified by one skilled in the art on the basis of sequence similarity using bioinformatics approaches. Typically BLAST (described above) searching of publicly available databases with known arabinose-proton symporter amino acid sequences, such as those provided herein, is used to identify additional arabinose-proton symporters, and their encoding sequences, that may be used in the present strains. These proteins may have at least about 80-85%, 85%-90%, 90%-95% or 95%-99% sequence identity to any of the arabinose-proton symporters of SEQ ID NOS:2, 4, 6, 8, 10, 12, 14, 16, 18, or 20 while having arabinose-proton symporter activity. Identities are based on the Clustal W method of alignment using the default parameters of GAP PENALTY=10, GAP LENGTH PENALTY=0.1, and Gonnet 250 series of protein weight matrix.

In addition to using protein or coding region sequence and bioinformatics methods to identify additional arabinose-proton symporters, the sequences described herein or those recited in the art may be used to experimentally identify other homologs in nature. For example each of the arabinose-proton symporter encoding nucleic acid fragments described herein may be used to isolate genes encoding homologous proteins. Isolation of homologous genes using sequence-dependent protocols is well known in the art. Examples of sequence-dependent protocols include, but are not limited to: 1.) methods of nucleic acid hybridization; 2.) methods of DNA and RNA amplification, as exemplified by various uses of nucleic acid amplification technologies [e.g., polymerase chain reaction (PCR), Mullis et al., U.S. Pat. No. 4,683,202; ligase chain reaction (LCR), Tabor, S. et al., Proc. Acad. Sci. USA 82:1074 (1985); or strand displacement amplification (SDA), Walker, et al., Proc. Natl. Acad. Sci. U.S.A., 89:392 (1992)]; and 3.) methods of library construction and screening by complementation.

For example, coding regions for similar proteins or polypeptides to the arabinose-proton symporter encoding sequences described herein could be isolated directly by using all or a portion of the instant nucleic acid fragments as DNA hybridization probes to screen libraries from any desired organism using methodology well known to those skilled in the art. Specific oligonucleotide probes based upon the disclosed nucleic acid sequences can be designed and synthesized by methods known in the art (Maniatis, supra). Moreover, the entire sequences can be used directly to synthesize DNA probes by methods known to the skilled artisan (e.g., random primers DNA labeling, nick translation or end-labeling techniques), or RNA probes using available in vitro transcription systems. In addition, specific primers can be designed and used to amplify a part of (or full-length of) the instant sequences. The resulting amplification products can be labeled directly during amplification reactions or labeled after amplification reactions, and used as probes to isolate full-length DNA fragments by hybridization under conditions of appropriate stringency.

Typically, in PCR-type amplification techniques, the primers have different sequences and are not complementary to each other. Depending on the desired test conditions, the sequences of the primers should be designed to provide for both efficient and faithful replication of the target nucleic acid. Methods of PCR primer design are common and well known in the art (Thein and Wallace, “The use of oligonucleotides as specific hybridization probes in the Diagnosis of Genetic Disorders”, in Human Genetic Diseases: A Practical Approach, K. E. Davis Ed., (1986) pp 33-50, IRL: Herndon, Va.; and Rychlik, W., In Methods in Molecular Biology, White, B. A. Ed., (1993) Vol. 15, pp 31-39, PCR Protocols: Current Methods and Applications. Humania: Totowa, N.J.).

Generally two short segments of the described sequences may be used in polymerase chain reaction protocols to amplify longer nucleic acid fragments encoding homologous genes from DNA or RNA. The polymerase chain reaction may also be performed on a library of cloned nucleic acid fragments wherein the sequence of one primer is derived from the described nucleic acid fragments, and the sequence of the other primer takes advantage of the presence of the polyadenylic acid tracts to the 3′ end of the mRNA precursor encoding microbial genes.

Alternatively, the second primer sequence may be based upon sequences derived from the cloning vector. For example, the skilled artisan can follow the RACE protocol (Frohman et al., PNAS USA 85:8998 (1988)) to generate cDNAs by using PCR to amplify copies of the region between a single point in the transcript and the 3′ or 5′ end. Primers oriented in the 3′ and 5′ directions can be designed from the instant sequences. Using commercially available 3′ RACE or 5′ RACE systems (e.g., BRL, Gaithersburg, Md.), specific 3′ or 5′ cDNA fragments can be isolated (Ohara et al., PNAS USA 86:5673 (1989); Loh et al., Science 243:217 (1989)).

Alternatively, the described arabinose-proton symporter encoding sequences may be employed as hybridization reagents for the identification of homologs. The basic components of a nucleic acid hybridization test include a probe, a sample suspected of containing the gene or gene fragment of interest, and a specific hybridization method. Probes are typically single-stranded nucleic acid sequences that are complementary to the nucleic acid sequences to be detected. Probes are “hybridizable” to the nucleic acid sequence to be detected. The probe length can vary from 5 bases to tens of thousands of bases, and will depend upon the specific test to be done. Typically a probe length of about 15 bases to about 30 bases is suitable. Only part of the probe molecule need be complementary to the nucleic acid sequence to be detected. In addition, the complementarity between the probe and the target sequence need not be perfect. Hybridization does occur between imperfectly complementary molecules with the result that a certain fraction of the bases in the hybridized region are not paired with the proper complementary base.

Hybridization methods are well defined. Typically the probe and sample must be mixed under conditions that will permit nucleic acid hybridization. This involves contacting the probe and sample in the presence of an inorganic or organic salt under the proper concentration and temperature conditions. The probe and sample nucleic acids must be in contact for a long enough time that any possible hybridization between the probe and sample nucleic acid may occur. The concentration of probe or target in the mixture will determine the time necessary for hybridization to occur. The higher the probe or target concentration, the shorter the hybridization incubation time needed. Optionally, a chaotropic agent may be added. The chaotropic agent stabilizes nucleic acids by inhibiting nuclease activity. Furthermore, the chaotropic agent allows sensitive and stringent hybridization of short oligonucleotide probes at room temperature (Van Ness and Chen, Nucl. Acids Res. 19:5143-5151 (1991)). Suitable chaotropic agents include guanidinium chloride, guanidinium thiocyanate, sodium thiocyanate, lithium tetrachloroacetate, sodium perchlorate, rubidium tetrachloroacetate, potassium iodide and cesium trifluoroacetate, among others. Typically, the chaotropic agent will be present at a final concentration of about 3 M. If desired, one can add formamide to the hybridization mixture, typically 30-50% (v/v).

Various hybridization solutions can be employed. Typically, these comprise from about 20 to 60% volume, preferably 30%, of a polar organic solvent. A common hybridization solution employs about 30-50% v/v formamide, about 0.15 to 1 M sodium chloride, about 0.05 to 0.1 M buffers (e.g., sodium citrate, Tris-HCl, PIPES or HEPES (pH range about 6-9)), about 0.05 to 0.2% detergent (e.g., sodium dodecylsulfate), or between 0.5-20 mM EDTA, FICOLL (Pharmacia Inc.) (about 300-500 kdal), polyvinylpyrrolidone (about 250-500 kdal) and serum albumin. Also included in the typical hybridization solution will be unlabeled carrier nucleic acids from about 0.1 to 5 mg/mL, fragmented nucleic DNA (e.g., calf thymus or salmon sperm DNA, or yeast RNA), and optionally from about 0.5 to 2% wt/vol glycine. Other additives may also be included, such as volume exclusion agents that include a variety of polar water-soluble or swellable agents (e.g., polyethylene glycol), anionic polymers (e.g., polyacrylate or polymethylacrylate) and anionic saccharidic polymers (e.g., dextran sulfate).

Nucleic acid hybridization is adaptable to a variety of assay formats. One of the most suitable is the sandwich assay format. The sandwich assay is particularly adaptable to hybridization under non-denaturing conditions. A primary component of a sandwich-type assay is a solid support. The solid support has adsorbed to it or covalently coupled to it immobilized nucleic acid probe that is unlabeled and complementary to one portion of the sequence.

Expression of an arabinose-proton symporter is achieved by transforming with a sequence encoding an arabinose-proton symporter. As known in the art, there may be variations in DNA sequences encoding an amino acid sequence due to the degeneracy of the genetic code. The coding sequence may be codon-optimized for maximal expression in the target Zymomonas or Zymobacter host cell, as well known to one skilled in the art. Typically a chimeric gene including a promoter active in Zymomonas cells that is operably linked to the desired coding region, as well as a transcription terminator, is used for expression. Any promoter that is active in Zymomonas cells may be used, such as the examples cited above for expression of proteins for arabinose utilization. A chimeric gene constructed with a promoter and arabinose-symporter coding region is a heterologous gene for expression in Zymomonas or Zymobacter since the coding region is from a different organism as described above. Vectors for expression and/or integration are as described above for expression of proteins for arabinose utilization.

### Improved Ethanol Production

The present strains have improved arabinose utilization in media with arabinose as the only carbohydrate source and in media with mixed sugars including arabinose The present strains also have improved ethanol production. As compared to the parental strain prior to introduction of an arabinose-proton symporter expression gene, ethanol production of the strain expressing an arabinose-proton symporter is increased. The increase in ethanol production may vary depending on the media and growth conditions used in fermentation as well as the arabinose-proton symporter expressing strain used as the biocatalyst. Typically ethanol production may be increased by at least about 10%, and may be increased by about 10%, 12%, 16%, 18%, 20%, 24%, 28%, or more.

### Fermentation of Improved Arabinose-Utilizing Strain

An engineered arabinose-utilizing strain expressing an arabinose-proton symporter and genes or operons for expression of L-arabinose isomerase, L-ribulokinase, L-ribulose-5-phosphate-4-epimerase, transaldolase and transketolase may be used in fermentation to produce a product that is a natural product of the strain, or a product that the strain is engineered to produce. For example, Zymomonas mobilis and Zymobacter palmae are natural ethanolagens. Preferred are strains that also utilize xylose and are engineered in addition for expression of xylose isomerase and xylulokinase. As an example, production of ethanol by a Z. mobilis strain of the invention, that utilizes xylose and arabinose, is described. Z mobilis also utilizes glucose naturally.

For production of ethanol, recombinant xylose and arabinose-utilizing Z. mobilis expressing an arabinose-proton symporter is brought in contact with medium that contains arabinose. Typically the medium contains mixed sugars including arabinose, xylose, and glucose. The medium may contain biomass hydrolysate that includes these sugars that are derived from treated cellulosic or lignocellulosic biomass.

When the mixed sugars concentration is high such that growth is inhibited, the medium includes sorbitol, mannitol, or a mixture thereof as disclosed in commonly owned and co-pending US Patent Pub. #US20080081358 A1. Galactitol or ribitol may replace or be combined with sorbitol or mannitol. The Z. mobilis grows in the medium where fermentation occurs and ethanol is produced. The fermentation is run without supplemented air, oxygen, or other gases (which may include conditions such as anaerobic, microaerobic, or microaerophilic fermentation), for at least about 24 hours, and may be run for 30 or more hours. The timing to reach maximal ethanol production is variable, depending on the fermentation conditions. Typically, if inhibitors are present in the medium, a longer fermentation period is required. The fermentations may be run at temperatures that are between about 30° C. and about 37° C., at a pH of about 4.5 to about 7.5.

The present Z. mobilis may be grown in medium containing mixed sugars including arabinose in laboratory scale fermenters, and in scaled up fermentation where commercial quantities of ethanol are produced. Where commercial production of ethanol is desired, a variety of culture methodologies may be applied. For example, large-scale production from the present Z. mobilis strains may be produced by both batch and continuous culture methodologies. A classical batch culturing method is a closed system where the composition of the medium is set at the beginning of the culture and not subjected to artificial alterations during the culturing process. Thus, at the beginning of the culturing process the medium is inoculated with the desired organism and growth or metabolic activity is permitted to occur adding nothing to the system. Typically, however, a “batch” culture is batch with respect to the addition of carbon source and attempts are often made at controlling factors such as pH and oxygen concentration. In batch systems the metabolite and biomass compositions of the system change constantly up to the time the culture is terminated. Within batch cultures cells moderate through a static lag phase to a high growth log phase and finally to a stationary phase where growth rate is diminished or halted. If untreated, cells in the stationary phase will eventually die. Cells in log phase are often responsible for the bulk of production of end product or intermediate in some systems. Stationary or post-exponential phase production can be obtained in other systems.

A variation on the standard batch system is the Fed-Batch system. Fed-Batch culture processes are also suitable for growth of the present Z. mobilis strains and comprise a typical batch system with the exception that the substrate is added in increments as the culture progresses. Fed-Batch systems are useful when catabolite repression is apt to inhibit the metabolism of the cells and where it is desirable to have limited amounts of substrate in the medium. Measurement of the actual substrate concentration in Fed-Batch systems is difficult and is therefore estimated on the basis of the changes of measurable factors such as pH and the partial pressure of waste gases such as CO₂. Batch and Fed-Batch culturing methods are common and well known in the art and examples may be found in Biotechnology: A Textbook of Industrial Microbiology, Crueger, Crueger, and Brock, Second Edition (1989) Sinauer Associates, Inc., Sunderland, Mass., or Deshpande, Mukund V., Appl. Biochem. Biotechnol., 36, 227, (1992), herein incorporated by reference.

Commercial production of ethanol may also be accomplished with a continuous culture. Continuous cultures are open systems where a defined culture medium is added continuously to a bioreactor and an equal amount of conditioned medium is removed simultaneously for processing. Continuous cultures generally maintain the cells at a constant high liquid phase density where cells are primarily in log phase growth. Alternatively, continuous culture may be practiced with immobilized cells where carbon and nutrients are continuously added, and valuable products, by-products or waste products are continuously removed from the cell mass. Cell immobilization may be performed using a wide range of solid supports composed of natural and/or synthetic materials as is known to one skilled in the art.

Continuous or semi-continuous culture allows for the modulation of one factor or any number of factors that affect cell growth or end product concentration. For example, one method will maintain a limiting nutrient such as the carbon source or nitrogen level at a fixed rate and allow all other parameters to moderate. In other systems a number of factors affecting growth can be altered continuously while the cell concentration, measured by medium turbidity, is kept constant. Continuous systems strive to maintain steady state growth conditions and thus the cell loss due to medium being drawn off must be balanced against the cell growth rate in the culture. Methods of modulating nutrients and growth factors for continuous culture processes as well as techniques for maximizing the rate of product formation are well known in the art of industrial microbiology and a variety of methods are detailed by Brock, supra.

Particularly suitable for ethanol production is a fermentation regime as follows. The desired Z. mobilis strain of the present invention is grown in shake flasks in semi-complex medium at about 30° C. to about 37° C. with shaking at about 150 rpm in orbital shakers and then transferred to a 10 L seed fermentor containing similar medium. The seed culture is grown in the seed fermentor anaerobically until OD₆₀₀ is between 3 and 6, when it is transferred to the production fermentor where the fermentation parameters are optimized for ethanol production. Typical inoculum volumes transferred from the seed tank to the production tank range from about 2% to about 20% v/v. Typical fermentation medium contains minimal medium components such as potassium phosphate (1.0-10.0 g/L), ammonium sulfate (0-2.0 g/L), magnesium sulfate (0-5.0 g/L), a complex nitrogen source such as yeast extract or soy based products (0-10 gL). A final concentration of about 5 mM sorbitol or mannitol is present in the medium. Mixed sugars including arabinose and at least one additional sugar such as glucose (or sucrose), providing a carbon source, are continually added to the fermentation vessel on depletion of the initial batched carbon source (50-200 g/l) to maximize ethanol rate and titer. Carbon source feed rates are adjusted dynamically to ensure that the culture is not accumulating glucose in excess, which could lead to build up of toxic byproducts such as acetic acid. In order to maximize yield of ethanol produced from substrate utilized, biomass growth is restricted by the amount of phosphate that is either batched initially or that is fed during the course of the fermentation. The fermentation is controlled at pH 5.0-6.0 using caustic solution (such as ammonium hydroxide, potassium hydroxide, or sodium hydroxide) and either sulfuric or phosphoric acid.

The temperature of the fermentor is controlled at 30° C.-35° C. In order to minimize foaming, antifoam agents (any class—silicone based, organic based etc) are added to the vessel as needed. An antibiotic, for which there is an antibiotic resistant marker in the strain, such as kanamycin, may be used optionally to minimize contamination.

In addition, fermentation may be concurrent with saccharification using an SSF (simultaneous saccharification and fermentation) process. In this process sugars are produced from biomass as they are metabolized by the production biocatalyst.

Any set of conditions described above, and additionally variations in these conditions that are well known in the art, are suitable conditions for production of ethanol by an arabinose-utilizing recombinant Zymomonas or Zymobacter strain that is engineered to express an arabinose-proton symporter by introducing a heterologous coding region of an arabinose-proton symporter.

### EXAMPLES

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various uses and conditions.

### General Methods

Standard recombinant DNA and molecular cloning techniques used here are well known in the art and are described by Sambrook, J., Fritsch, E. F. and Maniatis, T., Molecular Cloning: A Laboratory Manual, 2^{nd} ed., Cold Spring Harbor Laboratory: Cold Spring Harbor, N.Y. (1989) (hereinafter “Maniatis”); and by Silhavy, T. J., Bennan, M. L. and Enquist, L. W., Experiments with Gene Fusions, Cold Spring Harbor Laboratory: Cold Spring Harbor, N.Y. (1984); and by Ausubel, F. M. et al., Current Protocols in Molecular Biology, published by Greene Publishing Assoc. and Wiley-Interscience, Hoboken, N.J. (1987).

The meaning of abbreviations is as follows: “kb” means kilobase(s), “bp” means base pairs, “nt” means nucleotide(s), “hr” means hour(s), “min” means minute(s), “sec” means second(s), “d” means day(s), “L” means liter(s), “ml” means milliliter(s), “4” means microliter(s), “μg” means microgram(s), “ng” means nanogram(s), “mM” means millimolar, “μM” means micromolar, “nm” means nanometer(s), “μmol” means micromole(s), “pmol” means picomole(s), “Cm” means chloramphenicol, “Cm^{r}” means chloramphenicol resistant, “Cm^{S}“ means chloramphenicol sensitive, “Sp^{r}” means spectinomycin resistance, “Sp^{S}“ means spectinomycin sensitive, “UTR” means untranslated region, “RBS” means ribosome binding site.

Primers were synthesized by Sigma (St. Luis, Mo.) unless otherwise specified

### Example 1

### Construction and Expression of Operon for Arabinose Utilization Proteins in Zymomonas

To engineer Zymomonas mobilis for arabinose utilization, the E. coli araA, araB, and araC coding regions were constructed in an operon with a Z. mobilis promoter and expressed on a plasmind in Z. mobilis cells. AraB, araA, and araD encode the proteins L-ribulose kinase, L-arabinose isomerase, and L-ribulose-5-phosphate-4-epimerase, respectively, which provide an arabinose assimilation pathway, in conjunction with transketolase and transaldolase activities (see FIG. 1).

1. Cloning E. Coli araBAD Coding Sequences and Z. Mobilis P_{Gap} Promoter

The araB, araA, and araD coding regions of E. coli (SEQ ID NOs:23, 21, and 25, respectively) are present in the araBAD operon. An araB-araA DNA fragment (araBA; SEQ ID NO:27) was prepared using oligonucleotide primers ara1 (SEQ ID NO:28) and ara2 (SEQ ID NO:29) which are forward and reverse primers, respectively. Primer ara1 adds the nucleotides CC before the start codon ATG of the araB coding region to create an NcoI site. Primer ara2 adds an XbaI site after the stop codon of the araA coding region. An araD DNA fragment (SEQ ID NO:30) was prepared using oligonucleotide primers ara3 (SEQ ID NO:31) and primer ara4 (SEQ ID NO:32) which are forward and reverse primers, respectively. Primer ara3 adds an Xba site at the 5′ end of the ribosome binding site (RBS) sequence 5′ to the araD coding region. Primer ara4 adds a HindIII site after the 3′ untranslated region (UTR) that is 3′ to the araD coding region. Each pair of primers was used in a standard PCR reaction, including 50 μl AccuPrime Pfx SuperMix (Invitrogene, Carlsbad, Calif.), 1 μl of 10 μM forward and reverse primers, and 2 μl (approx. 50 to 100 ng) E. coli genomic DNA prepared from MG1655 (ATCC# 700926; a K12 strain) using a Wizard Genomic DNA Purification Kit (Promega, Madison, Wis.). A reaction using primers ara1 and ara2 was carried out for 5 min at 95° C., followed by 35 cycles of 30 sec at 95° C./30 sec at 56° C./3.5 min at 68° C., and ended for 7 min at 68° C. It resulted in a 3226-bp araB-araA fragment with a 5′ NcoI site and a 3′ XbaI site (SEQ ID NO:27). Another reaction using primers ara3 and ara4 was carried out using a similar program, except the extension time at 68° C. was shortened to 1.5 min. It produced an 889-bp araD fragment (including the araD 3′ UTR) with a 5′ XbaI site and a 3′ HindIII site (SEQ ID NO:30).

The native E. coli promoter for the araBAD operon is an inducible promoter that is not suitable for the desired expression in Z. mobilis. The Z. mobilis GAP (Glyceraldehydes-3-phosphate dehydrogenase) promoter (P_{gap}; SEQ ID NO:33) was used since it is a strong constitutive promoter for expression in Z. mobilis. A DNA fragment containing the Z. mobilis P_{gap} was prepared using oligonucleotide primers ara10 and ara11. Primer ara10 (SEQ ID NO:34) is a forward primer that adds a SacI and an ApeI site at the 5′ end of the promoter DNA fragment. Primer ara11 (SEQ ID NO:35) is a reverse primer that changes the last two nucleotides of the promoter from AC to CC, thus it adds an NcoI site at the 3′ end of the promoter DNA fragment. These two primers were used in a standard PCR reaction, as described above, using a plasmid containing the P_{gap} as the DNA template to produce a 323-bp P_{gap} promoter DNA fragment with 5′ SacI and SpeI sites and a 3′ NcoI site (SEQ ID NO:36).

Each of these PCR products was cloned into the TOPO Blunt Zero Vector (Invitrogen, Calsbad, Calif.) by following the manufacturer's instructions. The resultant plasmids pTP-araB-araA, pTP-araD and pTP-P_{gap} were propagated in E. coli DH5a cells (Invitrogen) and each was prepared using a Qiagen DNA Miniprep Kit. Their sequences were confirmed by DNA sequencing.

2. Assembling P_{gap}-araBAD Operon in a Shuttle Vector

A P_{gap}-araBAD operon was assembled in a Zymomonas-E. coli shuttle vector called pZB188aada, which is based on the vector pZB188 (Zhang et al. (1995) Science 267:240-243; U.S. Pat. No. 5,514,583) which includes a 2,582 by Z. mobilis genomic DNA fragment containing a replication region allowing the vector to replicate in Zymomonas cells. In pZB188aada the tetracycline resistance cassette (Tc^{r}-cassette) of pZB188 was replaced with a spectinomycin resistance cassette (Spec^{r}-cassette). The Spec^{r}-cassette was generated by PCR using plasmid pHP15578 (Cahoon et al, (2003) Nature Biotechnology 21: 1082-1087) as a template and Primers 1 (SEQ ID NO:32 from CL4236) and 2 (SEQ ID NO:33 from CL4236). Plasmid pHP15578 contains the complete nucleotide sequence for the Spec^{r}-cassette and its promoter, which is based on the published sequence of the Tranposon Tn7 aadA gene (GenBank accession number X03043) that codes for 3′ (9)-O-nucleotidyltransferase.

Primer 1 (SEQ ID NO: 37):
CTACTCATTTatcgatGGAGCACAGGATGACGCCT
Primer 2 (SEQ ID NO: 38):
CATCTTACTacgcgtTGGCAGGTCAGCAAGTGCC

The underlined bases of Primer 1 (forward primer) hybridize just upstream from the promotor for the Spec^{r}-cassette (to nts 4-22 of GenBank accession number X03043), while the lower case letters correspond to a ClaI site that was added to the 5′ end of the primer. The underlined bases of Primer 2 (reverse primer) hybridize about 130 bases downstream from the stop codon for the Spec^{r}-cassette (to nts 1002-1020 of GenBank accession number X03043), while the lower case letters correspond to an AflIII site that was added to the 5′ end of the primer. The 1048 by PCR-generated Spec^{r}-cassette was double-digested with ClaI and AflIII, and the resulting DNA fragment was purified using the QIAquick PCR Purification Kit (Qiagen, Cat. No. 28104) and the vendor's recommended protocol. Plasmid pZB188 (isolated from E. coli SSC110 (dcm⁻, dam⁻) in order to obtain non-methylated plasmid DNA for cutting with ClaI (which is sensitive to dam methylation) was double-digested with ClaI and BssHII to remove the Tc^{r}-cassette, and the resulting large vector fragment was purified by agarose gel electrophoresis. This DNA fragment and the cleaned up PCR product were then ligated together, and the transformation reaction mixture was introduced into E. coli JM110 using chemically competent cells that were obtained from Stratagene (Cat. No. 200239). Note that BssHII and AflIII generate compatible “sticky ends”, but both sites are destroyed when they are ligated together. Transformants were plated on LB medium that contained spectinomycin (100 μg/ml) and grown at 37° C. A spectinomycin-resistant transformant that contained a plasmid with the correct size insert was identified by restriction digestion analysis with NotI and named pZB188/aada.

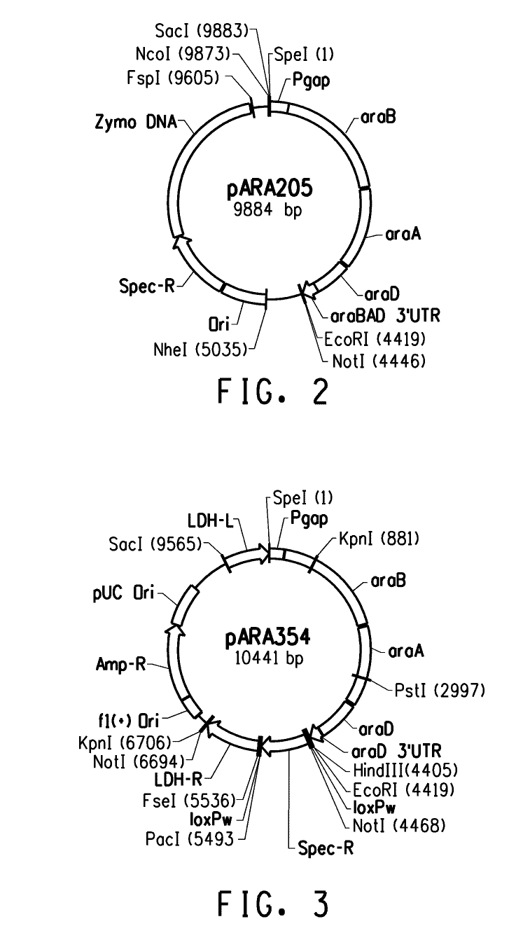

The pTP-P_{gap} SpeI-NcoI P_{gap} fragment, the pTP-araB-araA NcoI-XbaI araB-araA fragment, and the pTP-araD XbaI-NotI araD fragment were all cloned into a NotI-SpeI pZB188/aada vector, forming a pZB188aada-based shuttle vector that contained a P_{gap}-araBAD operon. The resulting plasmid, named pARA201, was propagated in E. coli DH5a and prepared using a Qiagen DNA Miniprep Kit. pARA205 (FIG. 2; SEQ ID NO:41) was prepared from pARA201 by restoring the nucleotides at the 3′ end of P_{gap} from CC back to the original AC nucleotides. This was done using a QickChange XL Site-Directed Mutagenesis Kit (Stratagene, La Jolla, Calif.). For this mutagenesis, the forward primer ara31 (SEQ ID NO:30) and the reverse primer ara32 (SEQ ID NO:40) were used to make the changes by following the manufacturer's instructions. pARA205 was propagated in E. coli DH5a and prepared using a Qiagen DNA Miniprep Kit.

3. Expressing araBAD in Z. Mobilis

To confirm that P_{gap}-araBAD is a functional operon in Z. mobilis, pARA205 was introduced into Z. mobilis strain ZW801-4 for expression. ZW801-4 is a xylose-utilizing strain of Z. mobilis. The construction and characterization of strains ZW658, ZW800 and ZW801-4 was described in commonly owned and co-pending U.S. Patent Application Publication US20080286870 A1, which is herein incorporated by reference. ZW658 (ATCC # PTA-7858) was constructed by integrating two operons, P_{gap}xylAB and P_{gap}taltkt, containing four xylose-utilizing genes encoding xylose isomerase, xylulokinase, transaldolase and transketolase, into the genome of ZW1 (ATCC #31821) via sequential transposition events, and followed by adaptation on selective media containing xylose. ZW800 is a derivative of ZW658 which has a double-crossover insertion of a spectinomycin resistance cassette in the sequence encoding the glucose-fructose oxidoreductase (GFOR) enzyme to knockout this activity. ZW801-4 is a derivative of ZW800 in which the spectinomycin resistance cassette was deleted by site-specific recombination leaving an in-frame stop codon that prematurely truncates the protein.

Competent cells of ZW801-4 were prepared by growing the seed cells overnight in MRM3G5 (1% yeast extract, 15 mM KH₂PO₄, 4 mM MgSO₄, and 50 g/L glucose) at 30° C. with 150 rpm shaking, up to an OD₆₀₀ value near 5. Cells were harvested and resuspended in fresh medium to an OD₆₀₀ value of 0.05. They were grown further under the same conditions to early or middle log phase (OD₆₀₀ near 0.5). Cells were harvested and washed twice with ice-cold water and then once with ice-cold 10% glycerol. The resultant competent cells were collected and resuspended in ice-cold 10% glycerol to an OD₆₀₀ value near 100. Since transformation of Z. mobilis requires non-methylated DNA, pARA205 plasmid was transformed into E. coli SCS110 competent cells (Stratagene). One colony of transformed cells was grown in 10 mL LB-Amp100 (LB broth containing 100 mg/L ampicillin) overnight at 37° C. DNA was prepared from the 10 mL-culture, using a Qiagen DNA Miniprep Kit.

Approximately 500 ng of non-methylated pARA205 plasmid DNA was mixed with 50 μL of ZW801-4 competent cells in a 1 MM Electroporation Cuvette (VWR, West Chester, Pa.). The plasmid DNA was electroporated into the cells at 2.0 KV using a BT720 Transporater Plus (BTX-Genetronics, San Diego, Calif.). The transformed cells were recovered in 1 mL MMG5 medium (50 g/L glucose, 10 g/L yeast extract, 5 g/L tryptone, 2.5 g/L (NH₄)₂SO₄, 0.2 g/L K₂HPO₄, and 1 mM MgSO₄) for 4 hours at 30° C. and grown on MMG5-Spec250 plates (MMG5 with 250 mg/L spectinomycin and 15 g/L agar) for 2 days at 30° C., inside an anaerobic jar with an AnaeroPack (Mitsubishi Gas Chemical, New York, N.Y.). Individual colonies were streaked onto a MMA5-Spec250 plate (as same as MMG5-Spec250 but glucose was replaced by 50 g/L arabinose) and a new MMG5-Spec250 plate in duplicate. Under the same conditions as described above, the streaks grew well although growth on the MMA5-Spec250 plate took longer time. This indicated that the P_{gap}-araBAD operon was expressed.

Two streaks of the transformed cells growing on the MMG5-Spec250 plate (ZW801-ara205-4 and ZW801-ara205-5) were selected for a 72-hour growth assay. In the assay, cells from each streak were grown overnight in 2 mL MRM3G5-Spec250 (MRM3G5 with 250 mg/L spectinomycin) at 30° C. with 150 rpm shaking. Cells were harvested, washed with MRM3A5 (same as MRM3G5 but glucose was replaced by arabinose), and resuspended in MRM3A5-Spec250 (MRM3A5 containing 250 mg/L spectinomycin) to have a start OD₆₀₀ at 0.1. Four mL of the suspension were placed in a 14 mL capped Falcon tube and grown for 72 hours at 30° C. with 150 rpm shaking. At the end of growth, OD₆₀₀ was measured. Then, 1 mL of the culture was centrifuged at 10,000×g to remove cells. The supernatant was filtered through a 0.22 μm Costar Spin-X Centrifuge Tube Filter (Corning Inc, Corning, N.Y.) and analyzed by running through a BioRad Aminex HPX-A7H ion exclusion column (BioRad, Hercules, Calif.) with 0.01 N H₂SO₄ at a speed of 0.6 mL/min at 55° C. on an Agilent 1100 HPLC system (Agilent Technologies, Santa Clara, Calif.) to determine ethanol and sugar concentrations. In parallel, ZW801-4 was grown (without antibiotics) and analyzed as a control. The results given in Table 2 demonstrate that expression of araBAD enabled Z. mobilis ZW801-4 to grow and produce ethanol using arabinose as the sole carbon source.

TABLE 2
72-hour growth assay for ZW801-ara205 strains in MRM3A5

| Strain | Growth (OD₆₀₀) | Ethanol (g/L) | Arabinoase (g/L) |
|---|---|---|---|
| ZW801-4 | 0.106 | 0 | 51.20 |
| ZW801-ara205-4 | 1.75 | 7.22 | 33.15 |
| ZW801-ara205-5 | 1.96 | 10.68 | 27.16 |

### Example 2

### Integration of Arabinose Utilization Operon into the Z. mobilis Genome and

### Characterization of Resulting Strains

This example describes stable integration of the P_{gap}-araBAD operon into two xylose-utilizing strains of Z. mobilis.

1. Building P_{gap}-araBAD Operon into a Suicide Vector.

To integrate the P_{gap}-araBAD operon into the genome of Z. mobilis, a suicide vector for DCO (double cross over) homologous recombination was prepared. Besides P_{gap}-araBAD, this vector included DCO homologous recombination fragments to direct integration of P_{gap}-araBAD and an aadA gene to provide a selective marker for spectinomycin resistance. We chose the IdhA locus as the insertion site. Two IdhA DNA fragments for DCO, LDH-L and LDH-R, were synthesized by PCR using Z. mobilis ZW801-4 DNA as template. The reaction used AccuPrime Mix and followed the standard PCR procedure described in Example 1. The LDH-L DNA fragment was synthesized using forward primer ara20 (SEQ ID NO:42) and reverse primer ara21 (SEQ ID NO:43). The resulting product was an 895-bp DNA fragment including sequence 5′ to the IdhA coding region and nucleotides 1-493 of the IdhA coding region, with a 5′ SacI site and a 3′ SpeI site (SEQ ID NO:44). The LDH-R DNA fragment was synthesized using forward primer ara22 (SEQ ID NO:45) and reverse primer ara23 (SEQ ID NO:46). The resulting product was a 1169 by fragment including nucleotides 494-996 of the IdhA coding region and sequence 3′ to the IdhA coding region, with a 5′ EcoRI site and a 3′ NotI site (SEQ ID NO:47).

pBS SK(+) (a Bluescript plasmid; Stratagene) was used as a suicide vector since pBS vectors cannot replicate in Zymomonas. pARA354 (SEQ ID NO:49) was constructed by cloning the P_{gap}-araBAD operon of pARA205, the LDH-L fragment, and the LDH-R fragment into pBS SK(+). In addition a DNA fragment containing the aadA marker (for spectinomycin resistance) bounded by wild type LoxP sites (LoxPw-aadA-LoxPw fragment; SEQ ID NO:48) was included in pARA354. pARA354 has the P_{gap}-araBAD operon and LoxPw-aadA-LoxPw marker fragment located between the LDH-L and LDH-R sequences.

FIG. 3 shows a map of the 10,441 bp pARA354. It has an f1(+) origin and an ampicillin resistance gene for plasmid propagation in E. coli. Since LDH-L and LDH-R contained the first 493 base pairs and the remaining 503 base pairs of the IdhA coding sequence, respectively, pARA354 was designed to direct insertion of P_{gap}-araBAD and aadA into the IdhA coding sequence of Z. mobilis between nucleotides #493 and #494 by crossover recombination.

2. Developing the P_{gap}-araBAD Integration Strains

Z. mobilis strain ZW705 is an engineered strain of Z. mobilis, with improved xylose utilization in stress conditions that was derived from ZW801-4 by adaptation in continuous culture as described in co-pending and commonly owned U.S. patent application Ser. No. 12/641,642, which is herein incorporated by reference. ZW801-4 xylose-utilizing Zymomonas cells were continuously grown in medium comprising at least about 50 g/L xylose to produce a culture comprising ethanol, then ammonia and acetic acid were added creating a stress culture. The cells were further continuously grown in the stress culture and cells with improved xylose utilization were isolated, including the ZW705 strain.

To transform pARA354 into both ZW705 and ZW801-4 strains, 800 ng non-methylated plasmid DNA was electroporated into 50 μl competent cells prepared from each strain. DNA demethylation, competent cell preparation, and electroporation were performed as described in Example 1. Colonies of transformed cells of each strain were grown on a MMG5-Spec250 plate for 2 days at 30° C. inside an anaerobic jar with an AnaeroPack. Because pARA354 could not replicate in Z. mobilis, spectinomycin resistance indicated these colonies were integration strains. The colonies were streaked on to a new MMG5-Spec250 plate and a MMA5-Spec250 plate, in duplicate, and grown for 2 days and 4 days respectively. Their growth on the MMA5-Spec250 plate also indicated the integration. To further demonstrate the integration, the junctions between the P_{gap}-araBAD-aadA fragment and Z. mobilis genomic DNA were inspected by the standard 35-cycle PCR reaction, containing PCR Super Mix (Invitrogen), a pair of primers, and the tested transformed cells. One PCR cycle included 45 seconds denaturing at 95° C., 45 seconds annealing at 58° C., and 2 minutes extension at 72° C. Primer ara45 (SEQ ID NO:50) and primer ara42 (SEQ ID NO:51) were a forward primer located at upstream of the LDH-L sequence in the Z. mobilis genomic DNA and a reverse primer located in the araB gene of pARA354, respectively. This pair of primers amplified a 1694-bp fragment from all colonies inspected by PCR. Also used were primer ara46 (SEQ ID NO:52) and primer ara43 (SEQ ID NO:53) which area forward primer located in the aadA gene of pARA354 and a reverse primer located downstream of the LDH-R sequence in Z. mobilis genomic DNA, respectively. This pair of primers amplified a 1521-bp fragment from all colonies inspected by PCR. Therefore, the P_{gap}-araBAD-aadA fragment had been integrated into ZW801-4 and ZW705 genomes successfully by the DCO approach. Because DCO homologous recombination was a target specific integration, every colony resulting from the integration in ZW801-4 or ZW705 would have the identical genotype. A colony from each of the integrations was grown in 5 mL MRMG5-Spec250 overnight at 30° C. with 150 rpm shaking. Cells were collected by centrifugation, resuspended in 0.5 mL 50% glycerol, and then stored at −80° C. The strains were named ZW705-ara354 and ZW801-ara354.

To further improve function of the integrated P_{gap}-araBAD operon, the ZW705-ara354 strain was subjected to adaptation. For this purpose, an overnight culture of ZW705-ara354 was collected by centrifugation, washed with MRM3A5, and resuspended in MRM3A5-Spec250 with OD₆₀₀ at 0.1. Four mL of this suspension was placed in a 14 mL Falcon capped tube and grown for 72 hours in a 30° C. 150 rpm shaker, until the OD₆₀₀ was above 1. Then the culture was inoculated to a new falcon tube containing 4 mL fresh MRM3A5-Spec250 to reach a starting OD₆₀₀ near 0.1 for a second run of growth. Totally, 9 successive runs were completed. Each run brought the OD₆₀₀ from approximately 0.1 to above 1 and took 3 to 4 days, except the 4^{th} run which took 6 days since the cells grew much more slowly. In order to characterize the adapted strains, the 9^{th} run was diluted 100-fold, and 10 μl of the dilution was spread and grown on a MMA5-Spec250 plate for 3 days at 30° C. in an anaerobic jar with an AnaeroPack. Individual colonies (i.e. adaptation strains) were picked and grown overnight in 3 mL MRM3G5-Spec250 on a 30° C. 150 rpm shaker. They were subjected to the 72-hour growth assay in MRM3A5-Spec250, as described in Example 1. ZW705-ara354 strain was used as a control in the assay. Analysis data for 5 adaptation strains (ZW705-ara354A4 to A8) are presented in Table 3, showing that all adaptation strains performed better than ZW705-ara354. ZW705-ara354A7 was the best strain in terms of growth, ethanol production, and arabinose utilization.

TABLE 3
72-hour growth assay for adaptation strains of ZW705-ara354 in
MRM3A5

| Strain | Growth (OD₆₀₀) | Ethanol (g/L) | Arabinoase (g/L) |
|---|---|---|---|
| ZW705-ara354 | 1.03 | 9.10 | 32.71 |
| ZW705-ara354A4 | 3.29 | 19.03 | 10.31 |
| ZW705-ara354A5 | 3.71 | 18.56 | 10.07 |
| ZW705-ara354A6 | 3.61 | 18.47 | 9.23 |
| ZW705-ara354A7 | 4.04 | 19.73 | 7.36 |
| ZW705-ara354A8 | 2.96 | 17.37 | 12.18 |

3. Characterizing Growth and Metabolite Profiles of the P_{gap}-araBAD Integration Strains, with and without Adaptation.

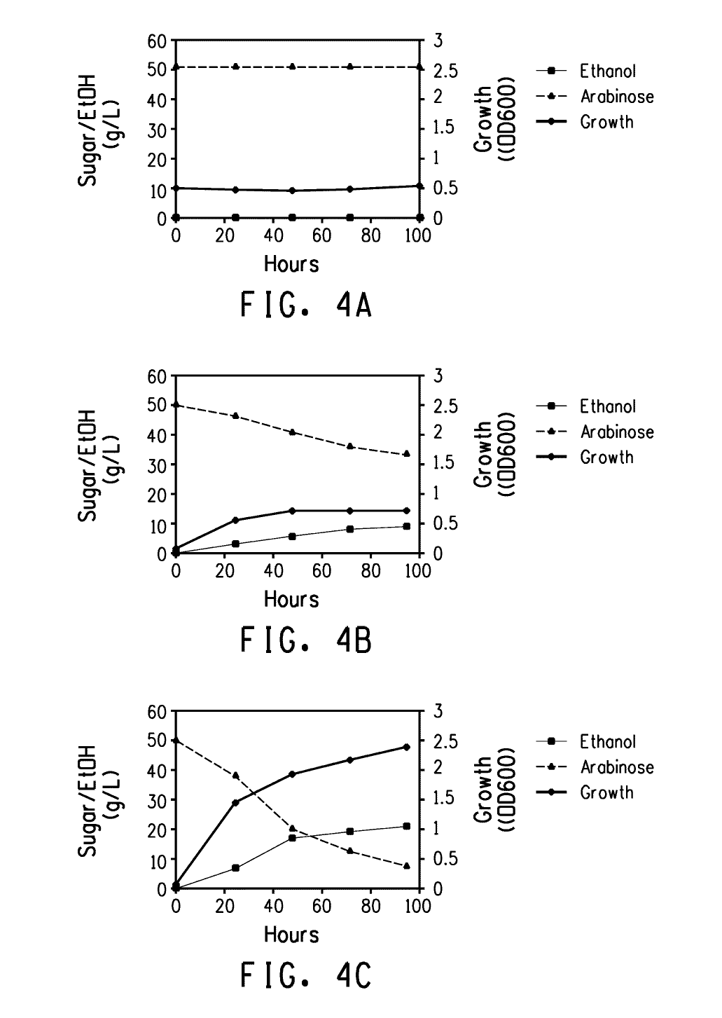

The P_{gap}-araBAD integration strains were further characterized for their ability to utilize arabinose to support cell growth and ethanol production in media containing arabinose as the sole carbon source and in media containing mixed sugars. To characterize these strains in medium containing arabinose as the sole carbon source, first ZW705-ara354 and ZW705-ara354A7 cells were grown overnight in 2 mL MRM3G5-Spec250 in a 30° C. 150 rpm shaker. Cells were harvested, washed with MRM3A5, and resuspended in MRM3A5-Spec250 at a starting OD₆₀₀ of 0.1. Twenty mL of the suspension were placed in a 50 mL screw capped VWR centrifuge tube and grown at 30° C. with 150 rpm shaking for a 96-hour time course. During the time course, OD₆₀₀ was measured at 0-, 24-, 48-, 72-, and 96-hour, respectively. At each time point, 1 mL of culture was to removed and centrifuged at 10,000×g to remove cells. The supernatant was filtered through a 0.22 μm Costar Spin-X Centrifuge Tube Filter and analyzed for ethanol and sugar concentrations by running through a BioRad Aminex HPX-A7H ion exclusion column with 0.01 N H₂SO₄ using a speed of 0.6 mL/min at 55° C. on an Agilent 1100 HPLC system. In parallel, ZW705 was grown in media without antibiotics and analyzed as a control. The results are given in FIG. 4. These results indicate that, without P_{gap}-araBAD, ZW705 could not metabolize arabinose and could not grow when arabinose was the sole carbon source (FIG. 4A). After integration of P_{gap}-araBAD, ZW705-ara354 was able to utilize arabinose to support growth and produce ethanol (FIG. 4B). The maximum rate of arabinose consumption was 0.2 g/L/hr. At the end of the time course, arabinose concentration in the medium was reduced by 32.8%, to 34 g/L. Adaptation greatly improved arabinose utilization, cell growth and ethanol production in ZW705-ara354A7. The maximum rate of arabinose consumption was 0.73 g/L/hr. At the end of time the course, arabinose concentration in the medium was reduced by 83.4%, to 8.4 g/L.

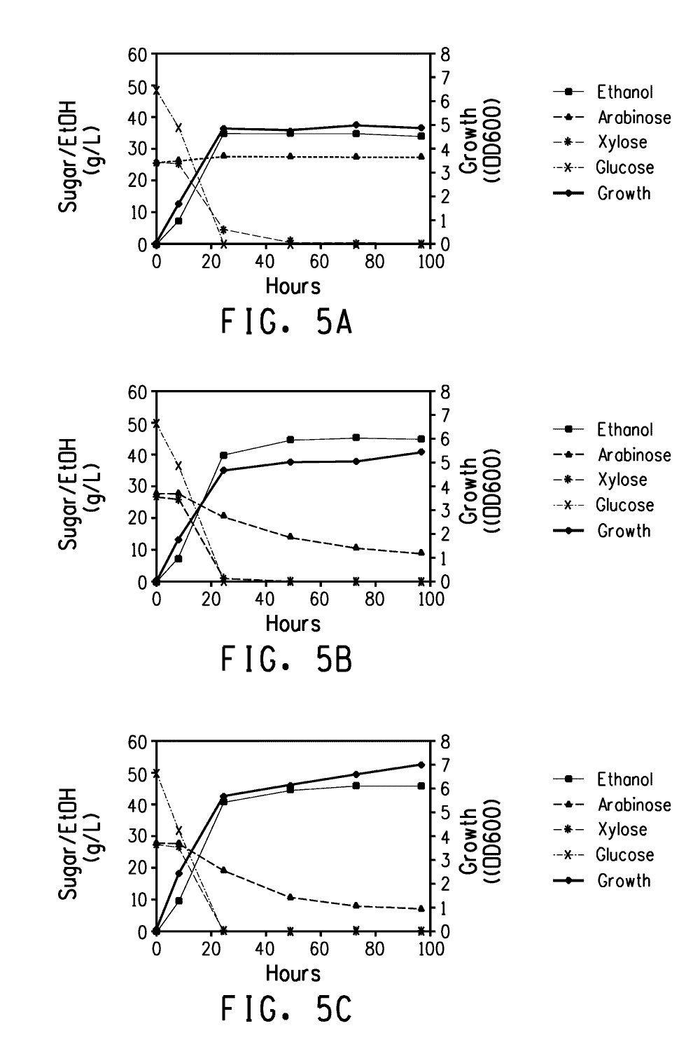

To characterize the strains in a medium containing mixed sugars, ZW705, ZW705-ara354, and ZW705-ara354A7 were grown and analyzed as described above, but the MRM3A5 media used in the previous experiment was replaced by MRM3A2.5X2.5G5 media(MRM3 with 25 g/L arabinose, 25 g/L xylose, and 50 g/L glucose). Due to fast growth in MRM3A2.5X2.5G5, a time point at 10 hour was added. Analysis was as described above for the experiment using arabinose medium. The results are given in FIG. 5. These results show that ZW705 efficiently utilized glucose and xylose to support strong cell growth and ethanol production, but it could not metabolize arabinose (FIG. 5A). After integration of P_{gap}-ara BAD, ZW705-ara354 was able to utilize arabinose to enhance cell growth and ethanol production (FIG. 5B). The maximum rate of arabinose consumption was 0.3 g/L/hr. At the end of the time course, arabinose concentration in the medium was reduced by 67.9%, to 8.8 g/L. In the adapted strain ZW705-ara354A7 there was some improvement over the ZW705-ara354 strain in arabinose utilization, which supported better growth and ethanol production. The maximum speed of arabinose consumption was 0.36 g/L/hr. At the end of the time course, arabinose concentration in the medium was reduced by 74.1%, to 7.1 g/L.

### Example 3

### Constructs for Expression of Two Arabinose Transport Systems from E. Coli in Zymomonas

Each of the two arabinose transport systems that are present in E. coli, encoded by araE or by araFGH, was expressed in Zymomonas and arabinose utilization analyzed. araE encodes an arabinose-proton symporter while araFGH encodes three proteins that form an ABC transporter.

1. Construction of Chimeric araE Gene and araFGH Operon for Expression in Zymomonas

E. coli araE and araFGH coding sequence DNA fragments were prepared by standard 30-cycle PCR, as described in Example 1, using E. coli MG1655 (a K12 strain: ATCC #700926) DNA as template. Each cycle included 45 sec denaturing at 94° C., 45 sec annealing at 60° C., and 4 min extension at 72° C. A forward primer ara135 (SEQ ID NO:54) and a reverse primer ara136 (SEQ ID NO:55) were used in PCR to synthesize a 1,550-bp araE fragment, including the araE coding sequence (1,419 bp) and its 3′UTR (121 bp), adding an NcoI site at the 5′ end and an EcoRI site at the 3′ end (SEQ ID NO:56). A forward primer ara137 (SEQ ID NO:57) and a reverse primer ara138 (SEQ ID NO:58) were used in PCR to synthesize a 3,744-bp araFGH fragment (SEQ ID NO:59). This fragment was identical to the E. coli araFGH operon but lacking the promoter. It included the araF coding sequence, araG coding sequence, araH coding sequence, araH 3′UTR, and intact intergenic regions. The primers added a 5′ NcoI site and a 3′ EcoRI site.

The Actinoplanes missouriensis GI promoter (P_{gi}) was chosen to direct the expression of araE and araFGH. It is the promoter of the xylose isomerase gene and has been demonstrated to function in Z. mobilis as a weak constitutive promoter. To clone A. missouriensis P_{gi}, a pair of oligonucleotide primers was designed. Primer ara12 (SEQ ID NO:60) was the forward primer for PCR of P_{gi}, which added a SacI and an SpeI site at the 5′ end of the promoter. Primer ara13 (SEQ ID NO:61) was the reverse primer for PCR of P_{gi}, which added an NcoI site at the 3′ end of the promoter. These two primers were used in a standard PCR reaction and a plasmid containing the Actinoplanes missouriensis GI promoter (SEQ ID NO:62) was used as template DNA. The PCR reaction produced a 201-bp P_{gi} DNA fragment (SEQ ID NO:63) with the 5′ SacI and SpeI sites and a 3′ NcoI site that was cloned into TOPO Blunt Zero Vector (Invitrogen, Calsbad, Calif.) by following the manufacturer's instructions. The resulting plasmid pTP-P_{g}, was propagated in E. coli DH5a and plasmid DNA prepared using a Qiagen DNA Miniprep Kit.

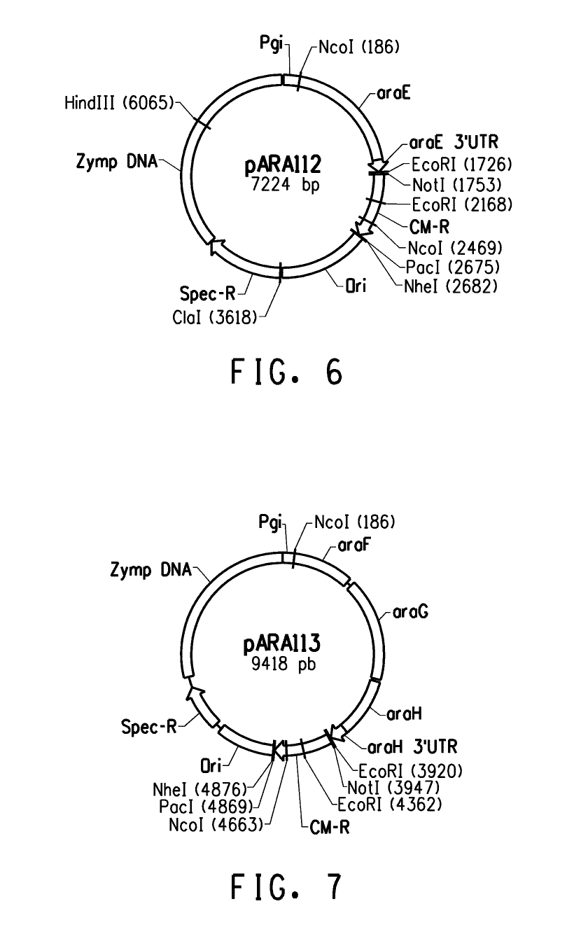

The SpeI-NcoI P_{gi}, fragment from pTP-P_{gi} and the NcoI-EcoRI araE PCR fragment were combined in a pZB188/aada vector along with a chloramphenicol resistance marker (CM-R; SEQ ID NO:64) creating pARA112 (FIG. 6; SEQ ID NO:65). pARA112 contains a P_{gi}-araE chimeric gene in the pZB188 derived E. coli/Zymomonas shuttle vector. The SpeI-NcoI P_{gi} fragment from pTP-P_{gi} and the NcoI-EcoRI araFGH PCR fragment were combined in a pZB188/aada vector along with a chloramphenicol resistance marker creating pARA113 (FIG. 7; SEQ ID NO:66). The resulting shuttle vectors were propagated in E. coli DH5a and plasmid DNA was prepared using a Qiagen DNA Miniprep Kit. The P_{gi}-araE gene and P_{gi}-araFGH operon were confirmed by sequencing.

### Example 4

### Expression of E. coli Arabinose Transport Systems in Zymomonas

### ZW705-ara354A7

Effects of the two arabinose transport systems of E. coli on arabinose utilizing Zymomonas cells were tested by expressing the constructed P_{gi}-araE gene and P_{gi}-araFGH operon.

1. Transforming ZW705-ara354A with pARA112 and pARA113.

pARA112 containing the P_{gi}-araE gene and pARA113 containing the P_{gi}-araFGH operon, both prepared in Example 3, were transformed into cells of ZW705-ara354A7 (prepared in Examples 1 and 2). Competent cells of the ZW705-ara354A7 strain were prepared as described in Example 1. Since tranformation of Z. mobilis requires non-methylated DNA, pARA112 and pARA113 were each transformed into E. coli SCS110 competant cells and non-methylated plasmid DNA was prepared from a 10 mL-culture of a single colony using a Qiagen DNA Miniprep Kit. Approximately 500 ng of each plasmid DNA was separately mixed with 50 μL ZW705-ara354A7 competant cells in a 1 MM VWR Electroporation Cuvette and electroporated into the cells at 2.0 KV using a BT720 Transporater Plus.

The pARA112 or pARA113 transformed cells (ZW705-ara354A7-ara112 and ZW705-ara354A7-ara113) were recovered in 1 mL MMG5 medium for 4 hours at 30° C. and then grown on MMG5-CM120 plates (MMG5 with 120 mg/L chloramphenicol and 15 g/L agar) for 2 days at 30° C. inside an anaerobic jar with an AnaeroPack. Individual colonies were streaked onto a new MMG5-CM120 plate and allowed to grow under the same conditions as in the last step. The streaks grew well on the chloramphenicol-containing plates, indicating successful transformation.

2. Expressing P_{gi}-araE and P_{gi}-araFGH in the Transformed Strains.

Several streaks of the transformed strains were selected from the MMG5-CM120 plates to represent ZW705-ara354A7-ara112 and ZW705-ara354A7-ara113. Expression of P_{gi}-araE or P_{gi}-araFGH was inspected by the 72-hour growth assay described in Example 1. In this assay, cells from each streak were grown overnight in 2 mL MRM3G5-CM120 (MRM3G5 with 120 mg/L chloramphenicol) at 30° C. with 150 rpm shaking. Cells were harvested, washed with MRM3A5, and resuspended in MRM3A5-CM120 (MRM3A5 containing 120 mg/L chloramphenicol) at a starting OD₆₀₀ of 0.1. Four mL of the suspension were grown for 72 hours at 30° C. with 150 rpm shaking. At the end of growth, OD₆₀₀ was measured and metabolite profiles were analyzed by using a BioRad Aminex HPX-A7H ion exclusion column on an Agilent 1100 HPLC system as described in Example 1. As a control, ZW705-ara354A7 strain was grown and analyzed in parallel with Spec250 replacing CM120. Results for 3 strains in each transformation are given in Table 4.

TABLE 4
72-hour growth assay for ZW705-ara354A7-ara112 and ZW705-
ara354A7-ara113 in MRM3A5.

| | | Ethanol | Arabinose |
|---|---|---|---|
| Strain | Growth (OD600) | (g/L) | (g/L) |
| ZW705-ara354A7 | 3.01 | 18.57 | 5.98 |
| ZW705-ara354A7-ara112-1 | 3.28 | 19.22 | 0.43 |
| ZW705-ara354A7-ara112-2 | 3.33 | 21.38 | 0.34 |
| ZW705-ara354A7-ara112-3 | 3.20 | 19.65 | 0.40 |
| ZW705-ara354A7-ara113-5 | 2.51 | 16.64 | 11.95 |
| ZW705-ara354A7-ara113-6 | 2.12 | 15.65 | 15.97 |
| ZW705-ara354A7-ara113-7 | 2.17 | 15.32 | 13.91 |

Comparing to their parent, all ZW705-ara354A7-ara112 strains utilized more arabinose during 72 hours growth, which supported a higher level of growth and ethanol production. In fact, these ZW705-ara354A7-ara112 strains had consumed almost all available arabinose in the medium. This indicates that araE facilitated arabinose utilization in the engineered strains. On the other hand, expression of araFGH appeared to have a negative impact. It resulted in less arabinose utilization, a lower level of growth and lower ethanol production in ZW705-ara354A7-ara113 strains during 72 hour growth.

3. Characterizing Growth and Metabolite Profiles of ZW705-ara354A7-ara112 Strain.

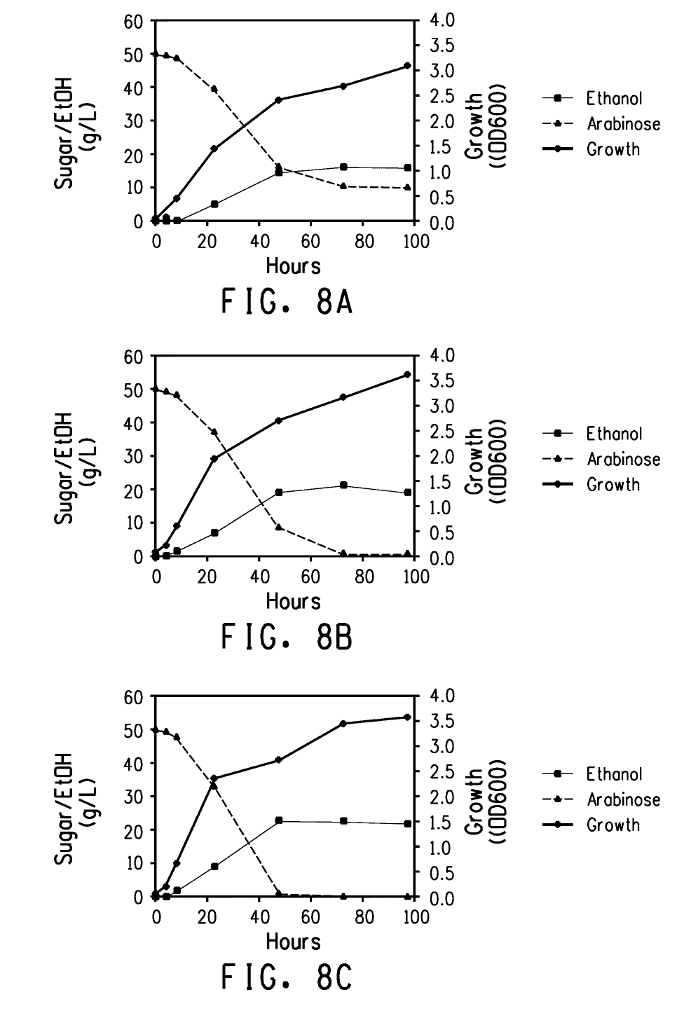

Since ZW705-ara354A7-ara112 strains showed facilitated arabinose metabolism, these strains were analyzed further. Characterization was preformed by following the procedure described in Example 2.3. Because araE was expressed from a shuttle vector, the expression level could vary between different strains. Therefore, two strains (ZW705-ara354A7-ara112-2 and ZW705-ara354A7-ara112-3) were examined side by side. To characterize strains in the single sugar (arabinose) medium, overnight grown ZW705-ara354A7-ara112-2 and ZW705-ara354A7-ara112-3 cultures were harvested, washed with MRM3A5, and resuspended in MRM3A5-CM120 to a starting OD₆₀₀ of 0.1. Twenty mL of the suspensions were grown at 30° C. with 150 rpm shaking for a 96-hour time course. OD₆₀₀ was measured at 0, 6, 12, 24, 48, 72, and 96 hour. At each time point, metabolite profiles were analyzed by using a BioRad Aminex HPX-A7H ion exclusion column on an Agilent 1100 HPLC system. In parallel, the parent strain ZW705-ara354A7 was grown in 250 mg/L spectinomycin instead 120 mg/L chloramphenicol and analyzed as a control. The results are given in FIG. 8. These results indicate that, without P_{gi}-araE, ZW705-ara354A7 utilized arabinose with a maximum speed of 0.93 g/L/hr. At the end of the time course, arabinose concentration in the medium was reduced by 80.4%, to 9.81 g/L. With expression of araE, ZW705-ara354A7-ara112-2 and ZW705-ara354A7-ara112-3 utilized arabinose more efficiently, which supported higher levels of growth and ethanol production. The maximum speeds of arabinose consumption increased to 1.18 g/L/hr and 1.28 g/L/hr in the 112-2 and 112-3 strains, respectively. At the end of the time course, arabinose concentration in the medium was reduced by 98%, to 1.02 g/L for ZW705-ara354A7-ara112-2 and by 99.2%, to 0.41 g/L for ZW705-ara354A7-ara112-3. In fact, ZW705-ara354A7-ara112-2 and ZW705-ara354A7-ara112-3 had almost exhausted all available arabinose after 72 hour and 48 hour culture, respectively.

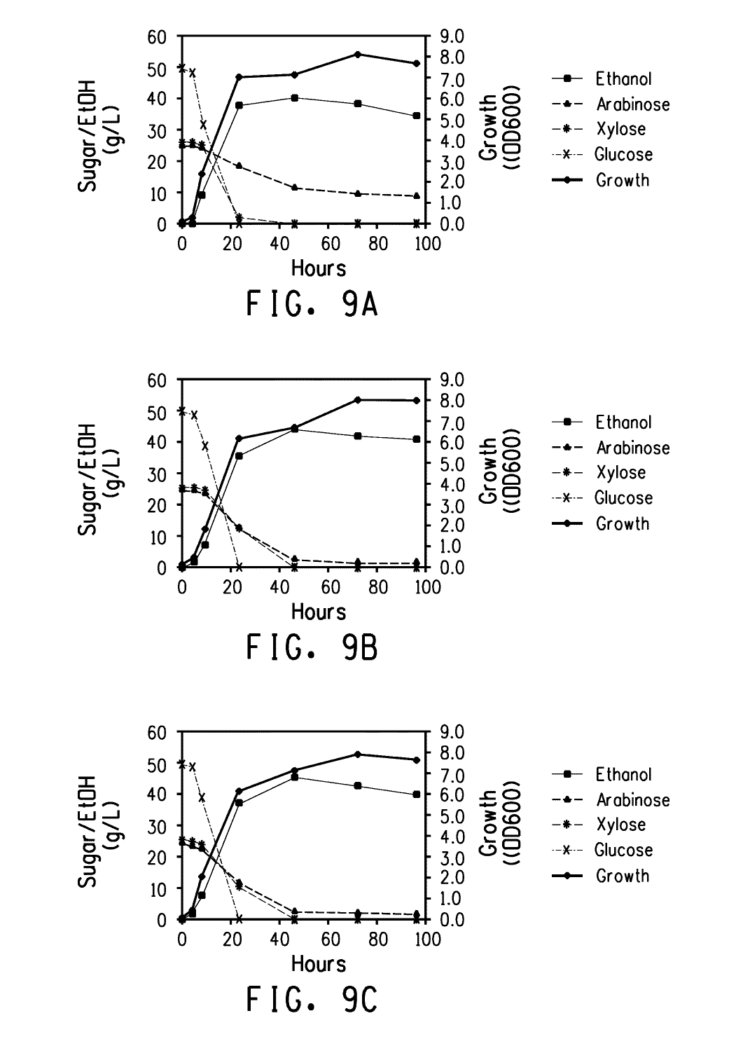

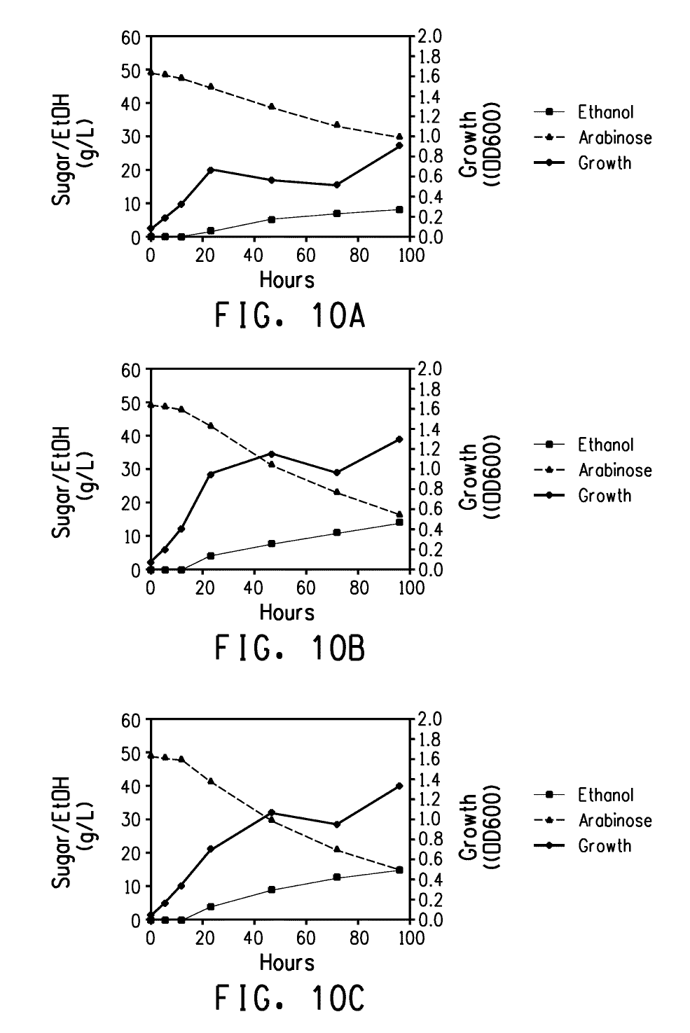

To characterize the strains in a medium containing mixed sugars, ZW705-ara354A7, ZW705-ara354A7-ara112-2, and ZW705-ara354A7-ara112-3 were grown and analyzed as described above but using MRM3A2.5X2.5G5 media. Results are given in FIG. 9. These results show that ZW705-ara354A7 efficiently exhausted all glucose and xylose within 24 hours to support strong growth and ethanol production. Its arabinose metabolism was relatively slower and incomplete. The maximum speed of arabinose consumption was 0.43 g/L/hr. At the end of time the course, arabinose concentration in the medium was reduced by 62.4%, to 9 g/L. However, ZW705-ara354A7-ara112-2 and ZW705-ara354A7-ara112-3 utilized arabinose much more efficiently. The maximum speeds of arabinose consumption increased to 0.73 g/L/hr and 0.78 g/L/hr, respectively. At the end of the time course, arabinose concentration in the medium was reduced by 90.3%, to 2.33 g/L for ZW705-ara354A7-ara112-2 and by 90.1%, to 2.38 g/L for ZW705-ara354A7-ara112-3. It had actually been reduced to near this level within 48 hours in both strains. Therefore, expression of araE had also facilitated arabinose utilization in the mixed sugar medium, which contributed to ethanol production as shown in FIG. 9. The expression had no significant effect on glucose metabolism, but it slowed down xylose metabolism so that both ZW705-ara354A7-ara112 strains took 48 hours to exhaust all xylose in the medium while the ZW705-ara354A7 strain took only 24 hours.

### Example 5

### Expression of araE in Zymomonas ZW705-ara354 and ZW801-ara354

In this example, effects of araE expression in non-adapted arabinose utilizing Z. mobilis strains ZW705-ara354 and ZW801-ara354 are analyzed.

1. Transforming ZW705-ara354 and ZW801-ara354 with pARA112.

As described in Example 2, ZW705-ara354 and ZW801-ara354 are engineered Z. mobilis strains developed from ZW705 and ZW801-4 by introducing P_{gap}-araBAD into the IdhA locus. ZW705-ara354 is the parental strain of ZW705-ara354A7 that was not adaptated in MRM3A5. Competent cells of both strains were prepared. Non-methylated DNA of pARA112 was electroporated into the competent cells as described in the previous examples.

The pARA112-transformed ZW705-ara354 (ZW705-ara354-ara112) and ZW801-ara354 ((ZW801-ara354-ara112) were recovered in 1 mL MMG5 medium for 4 hours at 30° C. and then grown on MMG5-CM120 plates for 2 days at 30° C. inside an anaerobic jar with an AnaeroPack. Individual colonies were streaked onto a new MMG5-CM120 plate and grown under the same conditions as in the last step. The streaks grew well on the chloramphenicol-containing plates, indicating successful transformation.

2. Expressing P_{gi}-araE in the Transformed Strains.

Several streaks of the transformed strains were selected from the MMG5-CM120 plates to represent ZW705-ara354-ara112 and ZW801-ara354-ara112, respectively. Expression of P_{gi}-araE was inspected by the 72-hour growth assay in MRM3A5. The details of assay were the same as in previous examples. As controls, ZW705-ara354 and ZW801-ara354 strains were grown and analyzed in parallel with 250 mg/L spectrinomycin replacing 120 mg/L chloramphenicol in the growth medium. The results for 3 strains from each transformation are given in Table 5. Compared to their parental strains, all ZW705-ara354-ara112 and ZW801-ara354-ara112 strains utilized significantly more arabinose during 72 hours growth, which supported a higher level of growth and ethanol production. Therefore, araE also facilitated arabinose utilization in the both ZW705-ara354-ara112 and ZW801-ara354-ara112 strains.

TABLE 5
72-hour growth assay for ZW705-ara354-ara112 and ZW801-
ara354-ara112 in MRM3A5

| | Growth | Ethanol | Arabinose |
|---|---|---|---|
| Strain | (OD600) | (g/L) | (g/L) |
| ZW705-ara354 | 1.15 | 9.56 | 27.88 |
| ZW705-ara354-ara112-1 | 1.56 | 14.18 | 17.24 |
| ZW705-ara354-ara112-2 | 1.67 | 16.71 | 10.93 |
| ZW705-ara354-ara112-3 | 1.47 | 13.76 | 19.06 |
| ZW801-ara354 | 1.39 | 9.65 | 27.08 |
| ZW801-ara354-ara112-4 | 1.95 | 15.01 | 15.12 |
| ZW801-ara354-ara112-5 | 2.07 | 15.51 | 12.94 |
| ZW801-ara354-ara112-5 | 2.29 | 15.79 | 13.05 |

3. Characterizing Growth and Metabolite Profiles of ZW705-ara354-ara112 and ZW801-ara354-ara112 Strains.

ZW705-ara354-ara112 and ZW801-ara354-ara112 strains were further characterized for their growth and metabolite profiles during a 96-hour time course. Characterization was performed by following the same procedure described in Example 4.3. ZW705-ara354-ara112-1 and ZW705-ara354-ara112-2 were examined and compared to their parent ZW705-ara354, while ZW801-ara354-ara112-5 and ZW801-ara354-ara112-6 were examined and compared to their parent ZW801-ara354. Measurement and analysis were done at 0, 6, 12, 24, 48, 72, and 96 hour time points.

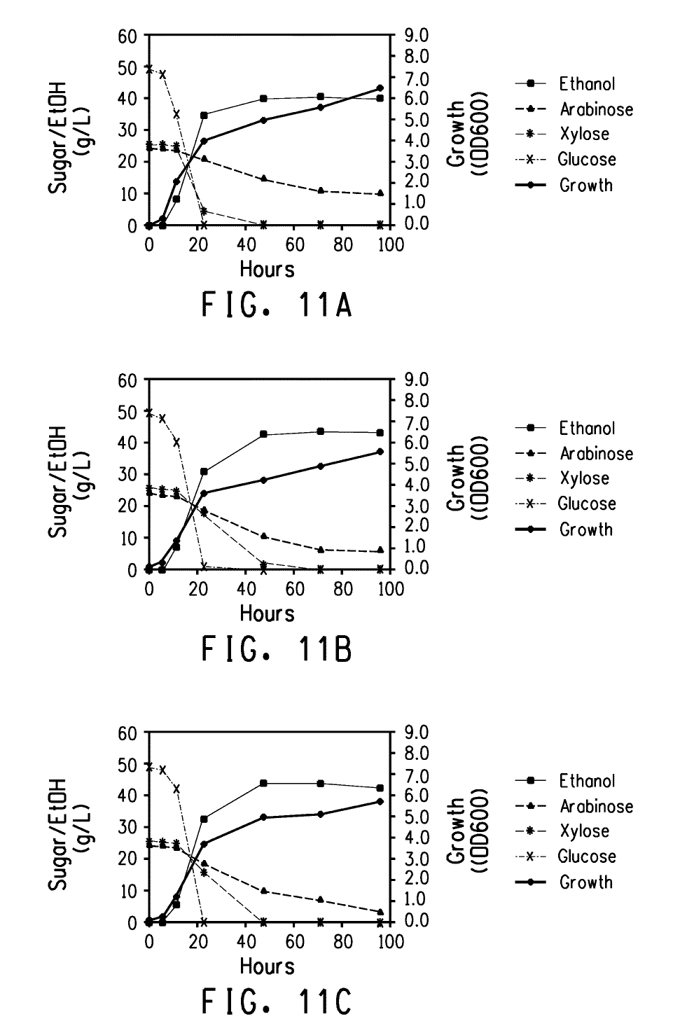

FIG. 10 shows the results obtained from ZW705-ara354 and ZW705-ara354-ara112 strains grown in MRM3A5. The results show that, without P_{gi}-araE, ZW705-ara354 utilized arabinose poorly, with a maximum rate of 0.25 g/L/hr. At the end of the time course, arabinose concentration in the medium was reduced by only 38.19%, to 30.22 g/L. With expression of araE, ZW705-ara354-ara112-1 and ZW705-ara354-ara112-2 utilized arabinose more efficiently, which supported higher levels of growth and ethanol production. The maximum rate of arabinose consumption increased to 0.46 g/L/hr and 0.48 g/L/hr, respectively. At the end of the time course, arabinose concentration in the medium was reduced by 65.8%, to 16.73 g/L for ZW705-ara354-ara112-1 and by 69.61%, to 14.86 g/L for ZW705-ara354-ara112-2.

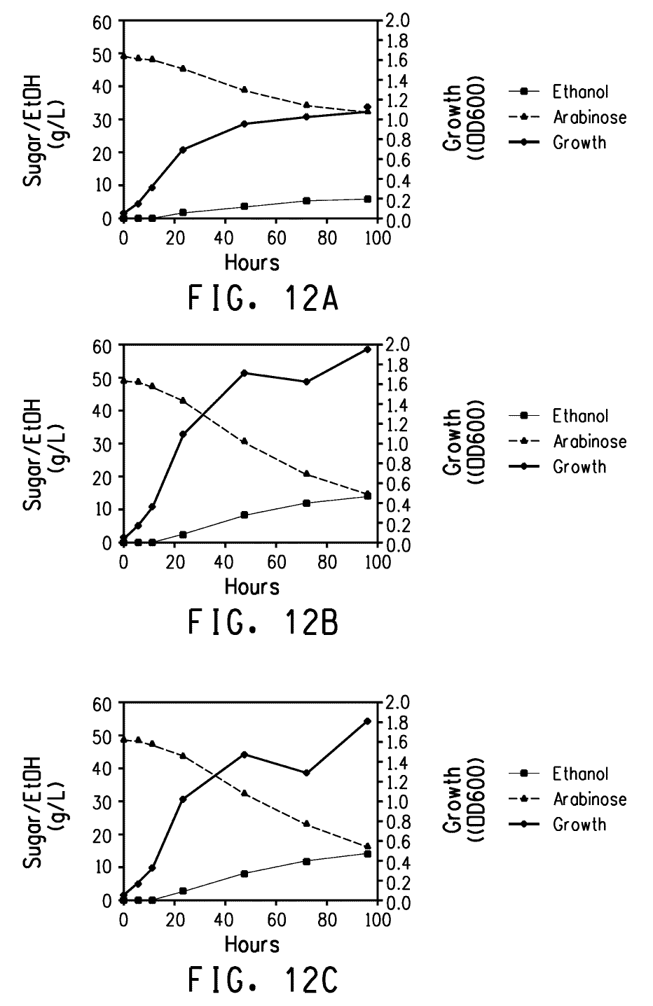

FIG. 11 shows the results obtained from ZW705-ara354 and

ZW705-ara354-ara112 strains grown in the mixed sugars medium MRM3A2.5X2.5G5. The results show that ZW705-ara354 efficiently used glucose and xylose to support strong growth and ethanol production. Its arabinose metabolism was slow and incomplete. The maximum rate of arabinose consumption was 0.29 g/L/hr. At the end of the time course, arabinose concentration in the medium was reduced by 57.32%, to 10.21 g/L. However, ZW705-ara354-ara112-1 and ZW705-ara354-ara112-2 utilized arabinose more efficiently. The maximum rate of arabinose consumption increased to 0.32 g/L/hr and 0.35 g/L/hr, respectively. At the end of the time course, arabinose concentration in the medium was reduced by 86.33%, to 3.27 g/L for ZW705-ara354-ara112-1 and by 85.2%, to 3.54 g/L for ZW705-ara354-ara112-2. These results demonstrated that expression of araE facilitated arabinose utilization in ZW705-ara354-ara112 strains in both single sugar medium (arabinose) and mixed sugar medium. Therefore, the araE effect did not require a genetic background acquired during the adaptation of ZW705-ara354A7. Similar to results in ZW705-ara354A7-ara112, the expression of araE slightly slowed down xylose metabolism in ZW705-ara354-ara112 grown in the mixed sugar medium.

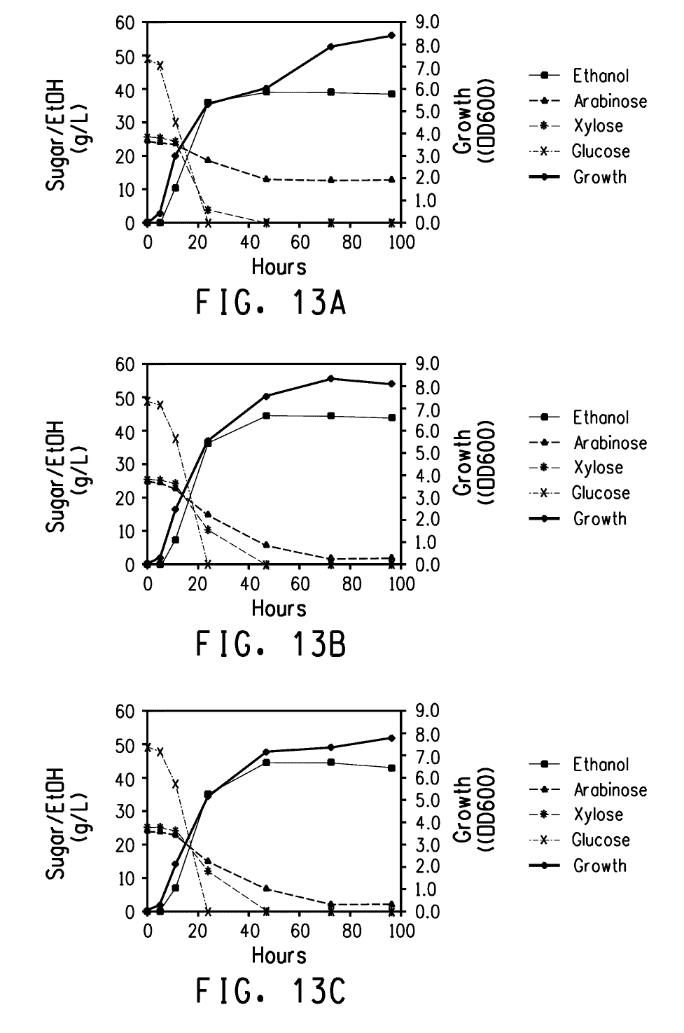

FIG. 12 shows the results obtained from ZW801-ara354 and

ZW801-ara354-ara112 strains growing in MRM3A5. The results indicate that, without P_{gi}-araE, ZW801-ara354 utilized arabinose poorly, with a maximum rate of 0.25 g/L/hr. At the end of the time course, arabinose concentration in the medium was reduced by only 32.99%, to 32.76 g/L. With expression of araE, ZW801-ara354-ara112-5 and ZW801-ara354-ara112-6 utilized arabinose more efficiently, which supported higher levels of growth and ethanol production. The maximum rate of arabinose consumption increased to 0.49 g/L/hr and 0.47 g/L/hr, respectively. At the end of the time course, arabinose concentration in the medium was reduced by 69.52%, to 14.90 g/L for ZW801-ara354-ara112-5 and by 65.92%, to 16.66 g/L for ZW801-ara354-ara112-6. FIG. 13 shows the results obtained from ZW801-ara354 and ZW801-ara354-ara112 strains grown in mixed sugar medium MRM3A2.5X2.5G5. It shows that ZW801-ara354 efficiently used glucose and xylose to support strong growth and ethanol production. Its arabinose metabolism was slow and incomplete. The maximum rate of arabinose consumption was 0.22 g/L/hr. At the end of the time course, arabinose concentration in the medium was reduced by 45.48%, to 13.04 g/L. However, ZW801-ara354-ara112-5 and ZW801-ara354-ara112-6 utilized arabinose more efficiently. The maximum rate of arabinose consumption increased to 0.35 g/L/hr and 0.36 g/L/hr, respectively. At the end of the time course, arabinose concentration in the medium was reduced by 89.92%, to 2.41 g/L for ZW801-ara354-ara112-5 and by 88.38%, to 2.78 g/L for ZW801-ara354-ara112-6. These results further demonstrated that expression of araE facilitated arabinose utilization in ZW801-ara354-ara112 strains in both single sugar medium and mixed sugar medium. Therefore, the araE effect was not limited to ZW705-ara354 and the derived strains. Similar to that in ZW705-ara354A7-ara112 and ZW705-ara354-ara112, the expression of araE slightly slowed down xylose metabolism in ZW801-ara354-ara112 grown in the mixed sugar medium.

What is claimed is:

1. A recombinant microorganism of the genus Zymomonas or Zymobacter that utilizes arabinose to produce ethanol, said microorganism comprising at least one heterologous gene encoding an arabinose-proton symporter.

2. The recombinant microorganism of claim 1 wherein the arabinose-proton symporter is encoded by the coding region of an araE gene.

3. The recombinant microorganism of claim 1 wherein arabinose utilization is improved by at least about 10% as compared to a parental microorganism wherein said parental microorganism is lacking the at least one heterologous gene encoding an arabinose-proton symporter.

4. The recombinant microorganism of claim 1 wherein the strain additionally utilizes xylose to produce ethanol.

5. A process for generating a recombinant microorganism of the genus Zymomonas or Zymobacter that has increased arabinose utilization comprising:
a) providing a recombinant Zymomonas or Zymobacter strain that utilizes arabinose to produce ethanol under suitable conditions; and
b) introducing at least one heterologous gene encoding an arabinose-proton symporter to the strain of (a).

6. The process according to claim 5, further comprising adapting the strain either before or after step (b), or both before and after step (b), by serial growth in media containing arabinose as the sole carbon source whereby an adapted strain is produced and wherein said stain has further improved arabinose utilization as compared to the strain with no adaptation.

7. The process according to claim 6, wherein the adapted strain additionally utilizes xylose and glucose for ethanol production in mixed sugars media comprising arabinose, xylose, and glucose.

8. A process for producing ethanol comprising:
a) providing a recombinant Zymomonas or Zymobacter strain that utilizes arabinose to produce ethanol, said strain comprising at least one heterologous gene encoding an arabinose-proton symporter; and
b) culturing the strain of (a) in a medium comprising arabinose whereby arabinose is converted to ethanol.

9. The process according to claim 9 wherein the arabinose-proton symporter is encoded by the coding region of an araE gene.

10. The process according to claim 8 wherein arabinose utilization is improved by at least about 10% as compared to a parental microorganism wherein said parental microorganism lacks a heterologous gene encoding an arabinose-proton symporter.

11. The process according to claim 8 wherein the strain of (a) is further capable of utilizing xylose and glucose to produce ethanol.

12. The process according to claim 8 wherein the strain of (a) has been adapted by serial growth in media containing arabinose as the sole carbon source whereby an arabinose-adapted strain is produced wherein said arabinose-adapted strain has increased ethanol production as compared to the strain of (a) that has not been adapted.

13. The process according to claim 8 wherein conversion of arabinose to ethanol is increased relative to conversion of arabinose to ethanol by a recombinant parental strain without at least one heterologous gene encoding an arabinose-proton symporter.

14. The process according to claim 13 wherein conversion of arabinose to ethanol is increased by at least about 10% as compared to a recombinant parental strain without at least one heterologous gene encoding an arabinose-proton symporter.

15. The process of claim 8 wherein the medium comprises either a mixture of sugars comprising arabinose or arabinose as a sole sugar.

16. A method for improving arabinose utilization by an arabinose-utilizing microorganism comprising:
(a) providing an arabinose-utilizing microorganism wherein said microorganism is selected from the group consisting of a recombinant Zymomonas or Zymobacter strain that utilizes arabinose to produce ethanol;
(b) introducing into the genome of said microorganism at least one heterologous gene encoding an arabinose-proton symporter wherein said symporter is expressed by said microorganism; and
(c) contacting the microorganism of (b) with a medium comprising arabinose, wherein said microorganism metabolizes said arabinose at an increased rate as compared to said microorganism that is lacking the arabinose-proton symporter.


SEQUENCE LISTING (66 sequences)

SEQ ID NO: 1 - DNA, 1416 bp, Escherichia coli
atggttactatcaatacggaatctgctttaacgccacgttctttgcgggatacgcggcgtatgaatatgtttgtttcggtagctgctgcggtcgcaggattgttatttggtcttgatatcggcgtaatcgccggagcgttgccgttcattaccgatcactttgtgctgaccagtcgtttgcaggaatgggtggttagtagcatgatgctcggtgcagcaattggtgcgctgtttaatggttggctgtcgttccgcctggggcgtaaatacagcctgatggcgggggccatcctgtttgtactcggttctatagggtccgcttttgcgaccagcgtagagatgttaatcgccgctcgtgtggtgctgggcattgctgtcgggatcgcgtcttacaccgctcctctgtatctttctgaaatggcaagtgaaaacgttcgcggtaagatgatcagtatgtaccagttgatggtcacactcggcatcgtgctggcgtttttatccgatacagcgttcagttatagcggtaactggcgcgcaatgttgggggttcttgctttaccagcagttctgctgattattctggtagtcttcctgccaaatagcccgcgctggctggcggaaaaggggcgtcatattgaggcggaagaagtattgcgtatgctgcgcgatacgtcggaaaaagcgcgagaagaactcaacgaaattcgtgaaagcctgaagttaaaacagggcggttgggcactgtttaagatcaaccgtaacgtccgtcgtgctgtgtttctcggtatgttgttgcaggcgatgcagcagtttaccggtatgaacatcatcatgtactacgcgccgcgtatcttcaaaatggcgggctttacgaccacagaacaacagatgattgcgactctggtcgtagggctgacctttatgttcgccacctttattgcggtgtttacggtagataaagcagggcgtaaaccggctctgaaaattggtttcagcgtgatggcgttaggcactctggtgctgggctattgcctgatgcagtttgataacggtacggcttccagtggcttgtcctggctctctgttggcatgacgatgatgtgtattgccggttatgcgatgagcgccgcgccagtggtgtggatcctgtgctctgaaattcagccgctgaaatgccgcgatttcggtattacctgttcgaccaccacgaactgggtgtcgaatatgattatcggcgcgaccttcctgacactgcttgatagcattggcgctgccggtacgttctggctctacactgcgctgaacattgcgtttgtgggcattactttctggctcattccggaaaccaaaaatgtcacgctggaacatatcgaacgcaaactgatggcaggcgagaagttgagaaatatcggcgtc

SEQ ID NO: 2 - protein, 472 aa, Escherichia coli
MVTINTESALTPRSLRDTRRMNMFVSVAAAVAGLLFGLDIGVIAGALPFITDHFVLTSRLQEWVVSSMMLGAAIGALFNGWLSFRLGRKYSLMAGAILFVLGSIGSAFATSVEMLIAARVVLGIAVGIASYTAPLYLSEMASENVRGKMISMYQLMVTLGIVLAFLSDTAFSYSGNWRAMLGVLALPAVLLIILVVFLPNSPRWLAEKGRHIEAEEVLRMLRDTSEKAREELNEIRESLKLKQGGWALFKINRNVRRAVFLGMLLQAMQQFTGMNIIMYYAPRIFKMAGFTTTEQQMIATLVVGLTFMFATFIAVFTVDKAGRKPALKIGFSVMALGTLVLGYCLMQFDNGTASSGLSWLSVGMTMMCIAGYAMSAAPVVWILCSEIQPLKCRDFGITCSTTTNWVSNMIIGATFLTLLDSIGAAGTFWLYTALNIAFVGITFWLIPETKNVTLEHIERKLMAGEKLRNIGV

SEQ ID NO: 3 - DNA, 1416 bp, Shigella flexneri
atggttactatcaatacggaatctgctttaacgccacgttctttgcgtgatacgcggcgtatgaatatgtttgtttcggtagctgctgcggtcgcaggattgttatttggtcttgatatcggcgtaatcgccggagcgttgccgttcattaccgatcactttgtgctgaccagtcgtttgcaggaatgggtggttagtagcatgatgctcggcgcagcaattggtgcgctgtttaatggttggctgtcgttccgcctggggcgtaaatacagcctgatggcgggggccatcctgtttgtactcggttctatagggtccgcttttgcgaccagcgtagagatgttaatcgccgctcgtgtggtgctgggcattgctgtcgggatcgcgtcttacaccgctcctctgtatctttctgaaatggcaagtgaaaacgttcgcggtaagatgatcagtatgtaccagttgatggtcacactcggcatcgtgctggcgtttttatccgatacagcgttcagttatagcggtaactggcgcgcaatgttgggggttcttgctttaccagcagttctgctgattattctggtggtcttcctgccaaatagcccgcgctggctggcggaaaaggggcgtcatattgaggcggaagaagtgttgcgtatgctgcgcgatacgtcggaaaaagcgcgagaagaactcaacgaaattcgtgaaagcctgaagttaaaacagggcggttgggcactgtttaagatcaaccgtaacgtccgtcgtgctgtgtttctcggtatgttgttgcaggcgatgcagcagtttaccggtatgaacatcatcatgtactacgcgccgcgtatcttcaaaatggcgggctttacgaccacagaacaacagatgattgcgactctggtcgtgggactgacctttatgttcgcgaccttcattgcggtctttacggtagataaagcaggtcgtaaaccggctctgaaaattggtttcagcgtgatggcgttaggcactctggtgctgggctattgcctgatgcagtttgataacggtacggcttccagtggcttgtcctggctctctgttggcatgacgatgatgtgtattgccggttatgcgatgagcgccgcgccagtggtgtggatcctgtgctctgaaattcagccgctgaaatgccgcgatttcggtattacctgttcgacgacgacaaactgggtgtcgaatatgattatcggcgcggccttcctgacactgcttgatagcattggcgctgccggtacgttctggctctacactgcgctgaacattgcgtttgtgggtattactttctggctcattccggaaaccaaaaatgtcacgctggaacatatcgaacgcaaactgatggcaggcgagaagttgagaaatatcggcgtc

SEQ ID NO: 4 - protein, 472 aa, Shigella flexneri
MVTINTESALTPRSLRDTRRMNMFVSVAAAVAGLLFGLDIGVIAGALPFITDHFVLTSRLQEWVVSSMMLGAAIGALFNGWLSFRLGRKYSLMAGAILFVLGSIGSAFATSVEMLIAARVVLGIAVGIASYTAPLYLSEMASENVRGKMISMYQLMVTLGIVLAFLSDTAFSYSGNWRAMLGVLALPAVLLIILVVFLPNSPRWLAEKGRHIEAEEVLRMLRDTSEKAREELNEIRESLKLKQGGWALFKINRNVRRAVFLGMLLQAMQQFTGMNIIMYYAPRIFKMAGFTTTEQQMIATLVVGLTFMFATFIAVFTVDKAGRKPALKIGFSVMALGTLVLGYCLMQFDNGTASSGLSWLSVGMTMMCIAGYAMSAAPVVWILCSEIQPLKCRDFGITCSTTTNWVSNMIIGAAFLTLLDSIGAAGTFWLYTALNIAFVGITFWLIPETKNVTLEHIERKLMAGEKLRNIGV

SEQ ID NO: 5 - DNA, 1416 bp, Shigella boydii
atggttactatcaatacggaatctgctttaacgccacgttctttgcgggatacgcggcgtatgaatatgtttgtttcggtagctgctgcggtcgcaggattgttatttggtcttgatatcggcgtaatcgccggagcgttgccgttcattaccgatcactttgtgctgaccagtcatttgcaggaatgggtggttagtagcatgatgctcggcgcagcaattggtgcgctgtttaatggttggctgtcgttccgcctggggcgtaaatacagcctgatggcgggggccatcctgtttgtactcggttctatagggtccgcttttgcgaccagcgtagagatgttaatcgccgctcgtgtggtgctgggcattgctgtcgggatcgcgtcttacaccgctcctctgtatctttctgaaatggcaagtgaaaacgttcgcggtaagatgatcagtatgtaccagttgatggtcacactcggcatcgtgctggcgtttttatccgatacagcgttcagttatagcggtaactggcgcgcaatgttgggggttcttgctttaccagcagttctgctgattattctggtggtcttcctgccaaatagcccgcgctggttggcggaaaaggggcgtcatattgaggcggaagaagtattgcgtatgctgcgcgatacgtcggaaaaagcgcgagaagaactcaacgaaattcgtgaaagcctgaagttaaaacagggcggttgggcactgtttaagatcaaccgtaacgtccgtcgtgctgtgtttctcggtatgttgttgcaggcgatgcagcagtttaccggtatgaacatcatcatgtactacgcgccgcgtatcttcaaaatggcgggctttacgaccacagaacaacagatgattgcgactctggtcgtagggctgacctttatgttcgccacctttattgcggtgtttacggtagataaagcagggcgtaaaccggctctgaaaattggtttcagcgtgatggcgttaggcactctggtgctgggctattgcctgatgcagtttgataacggtacggcttccagtggcttgtcctggctctctgttggcatgacgatgatgtgtattgccggttatgcgatgagcgccgcgccagtggtgtggatcctgtgctctgaaattcagccgctgaaatgccgcgatttcggtattacctgttcgaccaccacgaactgggtgtcgaatatgattatcggcgcgaccttcctgacgctgctcgacagcattggcgctgccggtacgttctggctctacactgcgctgaacattgcgtttgtgggcatcactttctggctcattccggaaaccaaaaatgtcacgctggaacatatcgaacgcaaactgatggcaggcgagaagttgagaaatatcggcatc

SEQ ID NO: 6 - protein, 472 aa, Shigella boydii
MVTINTESALTPRSLRDTRRMNMFVSVAAAVAGLLFGLDIGVIAGALPFITDHFVLTSHLQEWVVSSMMLGAAIGALFNGWLSFRLGRKYSLMAGAILFVLGSIGSAFATSVEMLIAARVVLGIAVGIASYTAPLYLSEMASENVRGKMISMYQLMVTLGIVLAFLSDTAFSYSGNWRAMLGVLALPAVLLIILVVFLPNSPRWLAEKGRHIEAEEVLRMLRDTSEKAREELNEIRESLKLKQGGWALFKINRNVRRAVFLGMLLQAMQQFTGMNIIMYYAPRIFKMAGFTTTEQQMIATLVVGLTFMFATFIAVFTVDKAGRKPALKIGFSVMALGTLVLGYCLMQFDNGTASSGLSWLSVGMTMMCIAGYAMSAAPVVWILCSEIQPLKCRDFGITCSTTTNWVSNMIIGATFLTLLDSIGAAGTFWLYTALNIAFVGITFWLIPETKNVTLEHIERKLMAGEKLRNIGI

SEQ ID NO: 7 - DNA, 1416 bp, Shigella dysenteriae
atggttactatcaatacggaatctgctttaacgccacgttctttgcgtgatacgcggcgtatgaatatgtttgtttcggtagctgctgcggtcgcaggattgttatttggtcttgatatcggcgtaatcgccggagcgttgccgttcattaccgatcactttgtgctgaccagtcgtttgcaggaatgggtggttagtagcatgatgctcggcgcagcaattggtgcgctgtttaatggttggctgtcgttccgcctggggcgtaaatacagcctgatggcgggggccatcctgtttgtactcggttctatagggtccgcttttgctaccagcgtagagatgttaatcgccgctcgtgtggtgctgggcattgctgtcgggatcgcgtcttacaccgctcctctgtatctttctgaaatggcaagtgaaaacgttcgcggtaagatgatcagtatgtaccagttgatggtcacactcggcatcgtgctggcgtttttatccgatacagcgttcagttatagcggtaactggcgcgcaatgttgggggttcttgctttaccagcagtcctgctgattattctggtggtcttcctgccaaatagcccgcgctggctggcggaaaaggggcgtcatattgaggcggaagaagtgttgcgtatgctgcgcgatacgtcggaaaaagcgcgagaagaactcaacgaaattcgtgaaagcctgaagttaaaacaaggcggttgggcactgtttaagatcaaccgtaacgtccgtcgtgctgtgtttctcggtatgttgttgcaggcgatgcagcagtttaccggtatgaacatcatcatgtactatgcgccgcgtatcttcaaaatggcgggctttacgaccacagaacaacagatgattgcgactctggtcgtgggactgacctttatgttcgcgaccttcattgcggtctttacggtagataaagcaggtcgtaaaccggctctgaaaattggtttcagcgtgatggcgttaggcactctggtgctgggctattgcctgatgcagtttgataacggtacggcttccagtggcttgtcctggctctctgttggcatgacgatgatgtgtattgccggttatgcgatgagcgccgcgccagtggtgtggatcctgtgctctgaaattcagccgctgaaatgccacgatttcggtattacctgttcgacgacgacaaactgggtgtcgaatatgattatcggcgcgaccttcctgacactgcttgatagcattggcgctgccggtacgttctggctctacactgcgctgaacattgcgtttgtgggcatcactttctggctcattccggaaaccaaaaatgtcacgctggaacatatcgaacgcaaactgatggcaggcgagaagttgagaaatatcggcgtc

SEQ ID NO: 8 - protein, 472 aa, Shigella dysenteriae
MVTINTESALTPRSLRDTRRMNMFVSVAAAVAGLLFGLDIGVIAGALPFITDHFVLTSRLQEWVVSSMMLGAAIGALFNGWLSFRLGRKYSLMAGAILFVLGSIGSAFATSVEMLIAARVVLGIAVGIASYTAPLYLSEMASENVRGKMISMYQLMVTLGIVLAFLSDTAFSYSGNWRAMLGVLALPAVLLIILVVFLPNSPRWLAEKGRHIEAEEVLRMLRDTSEKAREELNEIRESLKLKQGGWALFKINRNVRRAVFLGMLLQAMQQFTGMNIIMYYAPRIFKMAGFTTTEQQMIATLVVGLTFMFATFIAVFTVDKAGRKPALKIGFSVMALGTLVLGYCLMQFDNGTASSGLSWLSVGMTMMCIAGYAMSAAPVVWILCSEIQPLKCHDFGITCSTTTNWVSNMIIGATFLTLLDSIGAAGTFWLYTALNIAFVGITFWLIPETKNVTLEHIERKLMAGEKLRNIGV

SEQ ID NO: 9 - DNA, 1416 bp, Salmonella typhimurium
atggtctctattaatcatgactctgctttaacgccgcgttcgcttcgcgacacacgacgtatgaatatgtttgtttcggtttctgcagcggtagcgggactgttatttggtctggatatcggcgttatcgccggggcgctgccttttattaccgaccatttcgtactgaccagccggctgcaggaatgggtcgtcagcagcatgatgcttggcgcggcaattggcgcattatttaacggctggctttcattccggctggggcgtaagtatagcctgatggctggcgcgattttgttcgtgctcggctcgctggggtcggcgtttgcttccagcgtggaagtattgattggcgcccgcgtgatactgggcgtagcagtagggattgcctcctataccgcgccgctttatctctctgaaatggcaagtgaaaatgttcgcggcaaaatgatcagtatgtatcaactgatggtgacgttaggcattgtgctggcttttttatccgatacggcattcagctacagcggcaactggcgcgcgatgttgggcgtgctggcgctgcctgcggtgttgctcattattctggtggtattcctgccgaatagtccgcgttggctggcgcaaaaaggtcgccatattgaagcggaagaggtgctgcgtatgctgcgcgatacctcggaaaaagcccgtgatgaactgaatgagattcgggaaagcctcaaactcaagcagggagggtgggcattatttaaagctaaccgcaatgttcgccgcgccgtgttcctcggtatgctgctacaggcaatgcagcagttcaccggcatgaacatcattatgtactatgcgccgcgcatttttaaaatggccggctttaccaccacggaacagcaaatgatcgccacgctggtggtcggactgacttttatgttcgcgacgtttatcgccgtctttacggtcgataaggccgggcgtaaaccggcgttaaaaatcggtttcagcgtaatggcgttagggacattggtgttgggctactgcctgatgcagtttgataacggtacggcatcaagcggtctctcctggctttccgttgggatgacgatgatgtgtatcgccggttacgcgatgagcgccgctccggtggtgtggatactgtgttcggaaatccagccgctgaaatgccgtgattttggcattacctgttcaaccacgacaaactgggtatcgaacatgatcatcggcgcgacattcctgacactgttggacagcattggcgcggcaggtacattctggctctacaccgcgctgaatatcgcttttatcggcatcactttctggctgattccggaaaccaaaaatgtcaccctggagcacatcgaacgcaagctgatggcgggcgagaagctaagaaatattggcgtg

SEQ ID NO: 10 - protein, 472 aa, Salmonella typhimurium
MVSINHDSALTPRSLRDTRRMNMFVSVSAAVAGLLFGLDIGVIAGALPFITDHFVLTSRLQEWVVSSMMLGAAIGALFNGWLSFRLGRKYSLMAGAILFVLGSLGSAFASSVEVLIGARVILGVAVGIASYTAPLYLSEMASENVRGKMISMYQLMVTLGIVLAFLSDTAFSYSGNWRAMLGVLALPAVLLIILVVFLPNSPRWLAQKGRHIEAEEVLRMLRDTSEKARDELNEIRESLKLKQGGWALFKANRNVRRAVFLGMLLQAMQQFTGMNIIMYYAPRIFKMAGFTTTEQQMIATLVVGLTFMFATFIAVFTVDKAGRKPALKIGFSVMALGTLVLGYCLMQFDNGTASSGLSWLSVGMTMMCIAGYAMSAAPVVWILCSEIQPLKCRDFGITCSTTTNWVSNMIIGATFLTLLDSIGAAGTFWLYTALNIAFIGITFWLIPETKNVTLEHIERKLMAGEKLRNIGV

SEQ ID NO: 11 - DNA, 1431 bp, Salmonella enterica
ttgtggcaggaaaatatggtctctattaatcatgactctgctttaacgccgcgttcgcttcgcgacacacgacgtatgaatatgtttgtttcggtttctgcagcggtagcgggactgttatttggtctggatatcggcgttatcgccggggcgctgccttttattaccgaccatttcgtactgaccagccggctgcaggaatgggtcgtcagcagtatgatgcttggcgcggcaattggcgcattatttaacggctggctttcattccggctggggcgtaagtatagcctgatggctggcgcgattttgttcgtgctcggctcgctggggtcggcgtttgcttccagcgtggaagtattgattggcgcccgcgtgatactgggcgtagcagtagggattgcgtcctataccgcgccgctttatctctctgaaatggcaagtgaaaatgttcgcggcaaaatgatcagtatgtatcaactgatggtgacgttaggcattgtgctggcttttttatccgatacggcattcagctacagcggcaactggcgcgcgatgttgggcgtgctggcgctgcctgcggtgttgctcattattctcgtggtattcctgccgaatagtccgcgttggctggcgcaaaaaggtcgccatattgaagcggaagaggtgctgcgtatgctgcgcgatacctcggaaaaagcccgtgatgaactgaatgagattcgggaaagcctcaaactcaagcagggcgggtgggcattatttaaagctaaccgcaatgttcgccgcgccgtgttcctcggtatgctgctacaggcaatgcagcagttcaccggcatgaacatcattatgtactatgcgccgcgcatttttaaaatggccggctttaccaccacggaacagcaaatgatcgccacgctggtggtcggactgacctttatgttcgcgacgtttatcgccgtctttacggtcgataaggccgggcgtaaaccggcgttaaaaatcggtttcagcgtaatggcgttagggacattggtgttgggctactgcctgatgcagtttgataacggtacggcatcaagcggtctctcctggctttccgttgggatgacgatgatgtgtatcgccggttacgcgatgagcgccgctccggtggtgtggatactgtgttcggaaatccagccgctgaaatgccgtgattttggcattacctgttcaaccacgacaaactgggtatcgaacatgatcatcggcgcgacattcctgacactgttggacagtattggcgcggcaggtacattctggctctacaccgcgctgaatatcgcttttatcggcatcactttctggctgattccggaaaccaaaaatgtcaccctggagcatatcgaacgcaagctaatggcgggcgagaagctaagaaatattggcgtg

SEQ ID NO: 12 - protein, 477 aa, Salmonella enterica
MWQENMVSINHDSALTPRSLRDTRRMNMFVSVSAAVAGLLFGLDIGVIAGALPFITDHFVLTSRLQEWVVSSMMLGAAIGALFNGWLSFRLGRKYSLMAGAILFVLGSLGSAFASSVEVLIGARVILGVAVGIASYTAPLYLSEMASENVRGKMISMYQLMVTLGIVLAFLSDTAFSYSGNWRAMLGVLALPAVLLIILVVFLPNSPRWLAQKGRHIEAEEVLRMLRDTSEKARDELNEIRESLKLKQGGWALFKANRNVRRAVFLGMLLQAMQQFTGMNIIMYYAPRIFKMAGFTTTEQQMIATLVVGLTFMFATFIAVFTVDKAGRKPALKIGFSVMALGTLVLGYCLMQFDNGTASSGLSWLSVGMTMMCIAGYAMSAAPVVWILCSEIQPLKCRDFGITCSTTTNWVSNMIIGATFLTLLDSIGAAGTFWLYTALNIAFIGITFWLIPETKNVTLEHIERKLMAGEKLRNIGV

SEQ ID NO: 13 - DNA, 1419 bp, Klebsiella pneumoniae
atgacttcaatcagtaacgactctgcattaacgccgcggacacaacgtgacacccggcggatgaactggtttgtttctatcgctgcggcggtagcggggttgctctttggcctggatatcggcgtgatatccggggcgctgccctttattaccgaccacttcaccttatccagccagcttcaggagtgggtggtcagcagtatgatgttgggggcggcgatcggtgcgctgtttaacggctggctgtcgttccgcctcggccgtaaatacagcctgatggcgggggctgtgctctttgttgccggctctatcggctccgcttttgccgccagcgtggaggtgctgctgatagcccgcgtggtgttgggggtggccgtcgggatcgcttcctataccgcgccgttgtacctctccgagatggccagtgagaacgtgcgcgggaaaatgatcagtatgtaccagctgatggtgaccctcggcattgtgctggcgtttctttccgatactgcctttagctacagcggtaactggcgcgccatgttaggcgtgctggcactgccggcggtgatcctgattattctggtcgtctttttgccgaacagcccgcgctggctggcggagaaaggacgccatatcgaagcggaagaggtgctgcggatgctgcgcgatacctcggaaaaggcgcgcgacgagcttaacgagatccgtgagagcctgaagctgaagcagggcggctgggcgttgtttaaggtcaatcgtaacgtgcgccgggcggtgttccttggcatgctgctgcaggcgatgcagcagttcaccggcatgaacatcatcatgtactacgcgccgcgtatctttaaaatggcgggctttaccactaccgaacagcagatgatcgccaccctggtggtgggcctgacctttatgtttgccacctttattgcggtgttcacggtggataaagcgggtcgtaagccggcgctaaaaatcggctttagcgtgatggcgctgggcaccctggtgctgggctactgcctgatgcagttcgacaatggcaccgcctccagcggtctctcctggctttccgtcggcatgaccatgatgtgtattgccgggtatgcgatgagcgcggcgccggtggtgtggatcctctgctccgagatccagccgctgaaatgccgcgacttcggtatcacctgctcgaccaccaccaactgggtgtcgaacatgatcatcggcgccaccttcctgacgctgcttgacgcgattggcgccgccggcaccttctggctctacacggtgctcaacgtggcctttatcggcgtcaccttctggctgatcccggaaaccaagaatgtcaccctcgagcacattgagcgcaacctgatggcgggcgagaagctgcgcaacatcggtaaccgt

SEQ ID NO: 14 - protein, 473 aa, Klebsiella pneumoniae
MTSISNDSALTPRTQRDTRRMNWFVSIAAAVAGLLFGLDIGVISGALPFITDHFTLSSQLQEWVVSSMMLGAAIGALFNGWLSFRLGRKYSLMAGAVLFVAGSIGSAFAASVEVLLIARVVLGVAVGIASYTAPLYLSEMASENVRGKMISMYQLMVTLGIVLAFLSDTAFSYSGNWRAMLGVLALPAVILIILVVFLPNSPRWLAEKGRHIEAEEVLRMLRDTSEKARDELNEIRESLKLKQGGWALFKVNRNVRRAVFLGMLLQAMQQFTGMNIIMYYAPRIFKMAGFTTTEQQMIATLVVGLTFMFATFIAVFTVDKAGRKPALKIGFSVMALGTLVLGYCLMQFDNGTASSGLSWLSVGMTMMCIAGYAMSAAPVVWILCSEIQPLKCRDFGITCSTTTNWVSNMIIGATFLTLLDAIGAAGTFWLYTVLNVAFIGVTFWLIPETKNVTLEHIERNLMAGEKLRNIGNR

SEQ ID NO: 15 - DNA, 1416 bp, Klebsiella oxytoca
atgaccactctcagtcacgactctacaaccatgccgcgtacgcagcgcgatacccggcgcatgaatcagtttgtctccattgccgccgcggtggcagggttgctgtttggcctcgatatcggggtgattgccggggcgctgccctttattaccgaccattttgttttatccagccgcctgcaggagtgggtggtgagcagcatgatgctgggagccgccatcggcgcgttatttaacggctggctctctttccgcctcgggcgcaaatacagcctgatggtgggcgcggtgctgttcgttgccggctccgtgggctccgcgtttgcgaccagcgtcgaaatgctgctggtggcaaggatcgttctcggggtcgccgtggggatcgcctcttataccgcgccgctgtacctgtcggaaatggcgagcgaaaacgtgcgcggcaagatgatcagcatgtatcagctgatggtgacgctgggtatcgtgatggcgtttctctccgacaccgcgttcagctacagcggcaactggcgggcgatgcttggcgtactggcgctgccggcggtggtgctgattattctggtgatcttcctgccgaacagcccgcgctggctggcggaaaaagggcgtcacgtggaagcggaagaggtgctgcggatgctgcgcgacacgtcagaaaaagcccgtgacgagctcaacgagatccgcgaaagcctgaagctgaagcagggcggctgggcgctgtttaaggtcaaccgcaacgtgcggcgggcggtattcctcggcatgctgttgcaggcgatgcagcagtttaccggtatgaatatcatcatgtactacgcgccgcgcatctttaaaatggcgggcttcaccaccaccgaacagcagatggtcgcgaccctggtggttggcctgacctttatgttcgccacctttatcgccgtctttaccgtcgataaggccggacgtaagccggcgctgaaaatcggttttagcgtgatggccatcggcacgctggtgctgggctactgtctgatgcagtttgataacggcaccgcctccagcggtctctcctggctgtcggtggggatgaccatgatgtgtatcgccggctatgcgatgagcgccgcgccggtggtgtggatcctgtgttcggaaattcagccgctgaagtgccgcgatttcggcatcacctgctcaaccaccaccaactgggtgtcgaacatgattatcggcgcgaccttcctgacgctgctggacgcgatcggcgcggcaggaaccttctggctttataccgcgctgaacgtcgcctttatcggcgtgacgttctggctgatcccggaaaccaaaaacgtcaccctggagcatattgaacgcaggctgatgtccggcgagaagctgcgcaatatcggcaat

SEQ ID NO: 16 - protein, 472 aa, Klebsiella oxytoca
MTTLSHDSTTMPRTQRDTRRMNQFVSIAAAVAGLLFGLDIGVIAGALPFITDHFVLSSRLQEWVVSSMMLGAAIGALFNGWLSFRLGRKYSLMVGAVLFVAGSVGSAFATSVEMLLVARIVLGVAVGIASYTAPLYLSEMASENVRGKMISMYQLMVTLGIVMAFLSDTAFSYSGNWRAMLGVLALPAVVLIILVIFLPNSPRWLAEKGRHVEAEEVLRMLRDTSEKARDELNEIRESLKLKQGGWALFKVNRNVRRAVFLGMLLQAMQQFTGMNIIMYYAPRIFKMAGFTTTEQQMVATLVVGLTFMFATFIAVFTVDKAGRKPALKIGFSVMAIGTLVLGYCLMQFDNGTASSGLSWLSVGMTMMCIAGYAMSAAPVVWILCSEIQPLKCRDFGITCSTTTNWVSNMIIGATFLTLLDAIGAAGTFWLYTALNVAFIGVTFWLIPETKNVTLEHIERRLMSGEKLRNIGN

SEQ ID NO: 17 - DNA, 1413 bp, Enterobacter cancerogenus
atgacatctctcaatgactctaccctcatgcccgcggcgctgcgcgacacccgccgcatgaaccagtttgtctccgtcgcggcggccgtagcgggtctgctgtttgggctggatatcggcgttatcgccggtgcgctgccgtttatcaccgatcatttcacgttaagtcatcgcctgcaggagtgggtggtgagcagcatgatgctgggcgccgcaattggggcgttgttcaacggctggctctcgttccgcctgggacgaaagtacagcctgatggtcggggcgatcctgtttgtggccggttcactggggtcggcgtttgccacaagcgttgaggtgctgttgctctcccgcgtgctgcttggcgtggcggtggggatcgcctcctacaccgcgccgctgtatctctccgaaatggcgagcgagaacgtgcgcggcaagatgatcagcatgtatcagctgatggtgacgctcggcatcgtgctggcgtttctttccgatacctggttcagctacaccggtaactggcgcgccatgctcggcgtgctggcgttgcccgcgctgttgctgatggtgctggtgattttcctgccgaacagcccgcgctggctggcgcaaaaaggccgccacgtcgaggcggaagaagtgctgcgaatgctgcgtgacacctctgaaaaagcgcgtgaagagttgaacgagatccgcgaaagcctgaagctgaagcagggcggctgggcgctgtttaaggtcaaccgcaacgtgcgccgcgccgtgtttctgggaatgctcttgcaggcgatgcagcagtttacgggcatgaacatcatcatgtactacgccccgcgcatctttaaaatggcgggcttcaccacgaccgagcagcagatgatcgccaccctggtggtcgggctgacctttatgttcgccacctttattgccgtatttaccgtcgataaagccggacgtaaaccggcgctgaaaattggctttagcgtgatggcgctcggtacgctgatcctcggctactgcctgatgcagtttgatcagggcacggcatcgagcgggctttcctggctctccgtcggtatgaccatgatgtgcattgccggttatgcaatgagcgccgcgccggtggtgtggatcctgtgctctgaaattcagccgctaaaatgccgcgactttggtatcacctgttccaccaccaccaactgggtgtcgaacatgattatcggtgcgaccttcctgacgctgctggatgccattggtgcagcgggaacattctggctctacacggtgctgaacgtggcgtttattggcgtaacgttctggctgatcccagaaaccaaaggggtgacgctggagcacattgaacgcaagctgatggcgggggagaagttaaaaaacataggcgtg

SEQ ID NO: 18 - protein, 471 aa, Enterobacter cancerogenus
MTSLNDSTLMPAALRDTRRMNQFVSVAAAVAGLLFGLDIGVIAGALPFITDHFTLSHRLQEWVVSSMMLGAAIGALFNGWLSFRLGRKYSLMVGAILFVAGSLGSAFATSVEVLLLSRVLLGVAVGIASYTAPLYLSEMASENVRGKMISMYQLMVTLGIVLAFLSDTWFSYTGNWRAMLGVLALPALLLMVLVIFLPNSPRWLAQKGRHVEAEEVLRMLRDTSEKAREELNEIRESLKLKQGGWALFKVNRNVRRAVFLGMLLQAMQQFTGMNIIMYYAPRIFKMAGFTTTEQQMIATLVVGLTFMFATFIAVFTVDKAGRKPALKIGFSVMALGTLILGYCLMQFDQGTASSGLSWLSVGMTMMCIAGYAMSAAPVVWILCSEIQPLKCRDFGITCSTTTNWVSNMIIGATFLTLLDAIGAAGTFWLYTVLNVAFIGVTFWLIPETKGVTLEHIERKLMAGEKLKNIGV

SEQ ID NO: 19 - DNA, 1392 bp, Bacillus amyloliquefaciens
atgaagaatcacccggcaccaattggctcaaatgtacctgtcactcggcagcattccaagtggtttgtcattctcatctcatgcgcggccggactgggagggcttttgtacggttatgacacggcggtgatttccggcgctatcggtttcctgaaagatttgtaccgcttaagtccttttatggaagggctcgtgatttcaagcattatgatcggcggtgttttcggcgtcgggatttccggatttttgagtgaccgtttcggacggagaaagattttgatggcagcggcgctgttgtttgcggtgtcagcggttgtctctgcgctttctcaaagtgtgtcttccttagtgatcgccagagtcatcggcggtctgggaatcggcatgggctcctcgctttctgtcacgtatattaccgaagccgctccgccggccatacgcggcagtctgtcttcactgtatcagctgtttacgatattagggatatccggcacttattttattaaccttgccgtccagcagtccggctcgtatgaatggggagtgcacaccggctggcggtggatgctcgcttacggcatgattccgtccgtcatcttttttatcgtgctgcttatcgtgccggaaagtccgcgctggcttgcgaaagcggggcgccggaatgaagccctcgccgtgctgacgcgcattaacggcgagcagaccgcgaaagaagaaatcaaacaaatcgaaacgtctttacaattagaaaaaatgggttcattgtctcagctgtttaagccggggctgagaaaagcgcttgtgatcgggattctgctggctttattcaatcaggtcatcggcatgaacgcaattacgtattacgggccggaaattttcaaaatgatgggcttcggacagaatgcggggtttatcacgacatgcatcgtcggtgtcgttgaagtgattttcaccattatcgcggttcttttagtcgataaggtaggccggaaaaaactgatgggggtcggatctgcctttatggcgctgttcatgatcttaatcggggcatccttttattttcagctggcgagcggtccggctttagtcgtcatcatattgggattcgtcgccgctttctgcgtatcagtcgggccgattacatggatcatgatttcggaaatctttccgaaccacctccgcgcacgcgccgccggtattgcgacgatattcttatggggggcgaactgggcgatcggccagttcgtgccgatgatgatcagcgggttagggcttgcgtacaccttctggatattcgccgtcattaatattctctgtttcttgtttgtcgtgacgatctgccctgagacgaaaaataaatcattagaagaaatagaaaaactctggataaaa

SEQ ID NO: 20 - protein, 464 aa, Bacillus amyloliquefaciens
MKNHPAPIGSNVPVTRQHSKWFVILISCAAGLGGLLYGYDTAVISGAIGFLKDLYRLSPFMEGLVISSIMIGGVFGVGISGFLSDRFGRRKILMAAALLFAVSAVVSALSQSVSSLVIARVIGGLGIGMGSSLSVTYITEAAPPAIRGSLSSLYQLFTILGISGTYFINLAVQQSGSYEWGVHTGWRWMLAYGMIPSVIFFIVLLIVPESPRWLAKAGRRNEALAVLTRINGEQTAKEEIKQIETSLQLEKMGSLSQLFKPGLRKALVIGILLALFNQVIGMNAITYYGPEIFKMMGFGQNAGFITTCIVGVVEVIFTIIAVLLVDKVGRKKLMGVGSAFMALFMILIGASFYFQLASGPALVVIILGFVAAFCVSVGPITWIMISEIFPNHLRARAAGIATIFLWGANWAIGQFVPMMISGLGLAYTFWIFAVINILCFLFVVTICPETKNKSLEEIEKLWIK

SEQ ID NO: 21 - DNA, 1500 bp, Escherichia coli
atgacgatttttgataattatgaagtgtggtttgtcattggcagccagcatctgtatggcccggaaaccctgcgtcaggtcacccaacatgccgagcacgtcgttaatgcgctgaatacggaagcgaaactgccctgcaaactggtgttgaaaccgctgggcaccacgccggatgaaatcaccgctatttgccgcgacgcgaattacgacgatcgttgcgctggtctggtggtgtggctgcacaccttctccccggccaaaatgtggatcaacggcctgaccatgctcaacaaaccgttgctgcaattccacacccagttcaacgcggcgctgccgtgggacagtatcgatatggactttatgaacctgaaccagactgcacatggcggtcgcgagttcggcttcattggcgcgcgtatgcgtcagcaacatgccgtggttaccggtcactggcaggataaacaagcccatgagcgtatcggctcctggatgcgtcaggcggtctctaaacaggatacccgtcatctgaaagtctgccgatttggcgataacatgcgtgaagtggcggtcaccgatggcgataaagttgccgcacagatcaagttcggtttctccgtcaatacctgggcggttggcgatctggtgcaggtggtgaactccatcagcgacggcgatgttaacgcgctggtcgatgagtacgaaagctgctacaccatgacgcctgccacacaaatccacggcaaaaaacgacagaacgtgctggaagcggcgcgtattgagctggggatgaagcgtttcctggaacaaggtggcttccacgcgttcaccaccacctttgaagatttgcacggtctgaaacagcttcctggtctggccgtacagcgtctgatgcagcagggttacggctttgcgggcgaaggcgactggaaaactgccgccctgcttcgcatcatgaaggtgatgtcaaccggtctgcagggcggcacctcctttatggaggactacacctatcacttcgagaaaggtaatgacctggtgctcggctcccatatgctggaagtctgcccgtcgatcgccgcagaagagaaaccgatcctcgacgttcagcatctcggtattggtggtaaggacgatcctgcccgcctgatcttcaatacccaaaccggcccagcgattgtcgccagcttgattgatctcggcgatcgttaccgtctactggttaactgcatcgacacggtgaaaacaccgcactccctgccgaaactgccggtggcgaatgcgctgtggaaagcgcaaccggatctgccaactgcttccgaagcgtggatcctcgctggtggcgcgcaccataccgtcttcagccatgcactgaacctcaacgatatgcgccaattcgccgagatgcacgacattgaaatcacggtgattgataacgacacacgcctgccagcgtttaaagacgcgctgcgctggaacgaagtgtattacggatttcgtcgc

SEQ ID NO: 22 - protein, 500 aa, Escherichia coli
MTIFDNYEVWFVIGSQHLYGPETLRQVTQHAEHVVNALNTEAKLPCKLVLKPLGTTPDEITAICRDANYDDRCAGLVVWLHTFSPAKMWINGLTMLNKPLLQFHTQFNAALPWDSIDMDFMNLNQTAHGGREFGFIGARMRQQHAVVTGHWQDKQAHERIGSWMRQAVSKQDTRHLKVCRFGDNMREVAVTDGDKVAAQIKFGFSVNTWAVGDLVQVVNSISDGDVNALVDEYESCYTMTPATQIHGKKRQNVLEAARIELGMKRFLEQGGFHAFTTTFEDLHGLKQLPGLAVQRLMQQGYGFAGEGDWKTAALLRIMKVMSTGLQGGTSFMEDYTYHFEKGNDLVLGSHMLEVCPSIAAEEKPILDVQHLGIGGKDDPARLIFNTQTGPAIVASLIDLGDRYRLLVNCIDTVKTPHSLPKLPVANALWKAQPDLPTASEAWILAGGAHHTVFSHALNLNDMRQFAEMHDIEITVIDNDTRLPAFKDALRWNEVYYGFRR

SEQ ID NO: 23 - DNA, 1698 bp, Escherichia coli
atggcgattgcaattggcctcgattttggcagtgattctgtgcgagctttggcggtggactgcgctaccggtgaagagatcgccaccagcgtagagtggtatccccgttggcagaaagggcaattttgtgatgccccgaataaccagttccgtcatcatccgcgtgactacattgagtcaatggaagcggcactgaaaaccgtgcttgcagagcttagcgtcgaacagcgcgcagctgtggtcgggattggcgttgacagtaccggctcgacgcccgcaccgattgatgccgacggaaacgtgctggcgctgcgcccggagtttgccgaaaacccgaacgcgatgttcgtattgtggaaagaccacactgcggttgaagaagcggaagagattacccgtttgtgccacgcgccgggcaacgttgactactcccgctacattggtggtatttattccagcgaatggttctgggcaaaaatcctgcatgtgactcgccaggacagcgccgtggcgcaatctgccgcatcgtggattgagctgtgcgactgggtgccagctctgctttccggtaccacccgcccgcaggatattcgtcgcggacgttgcagcgccgggcataaatctctgtggcacgaaagctggggcggcctgccgccagccagtttctttgatgagctggacccgatcctcaatcgccatttgccttccccgctgttcactgacacttggactgccgatattccggtgggcaccttatgcccggaatgggcgcagcgtctcggcctgcctgaaagcgtggtgatttccggcggcgcgtttgactgccatatgggcgcagttggcgcaggcgcacagcctaacgcactggtaaaagttatcggtacttccacctgcgacattctgattgccgacaaacagagcgttggcgagcgggcagttaaaggtatttgcggtcaggttgatggcagcgtggtgcctggatttatcggtctggaagcaggccaatcggcgtttggtgatatctacgcctggtttggtcgcgtactcggctggccgctggaacagcttgccgcccagcatccggaactgaaaacgcaaatcaacgccagccagaaacaactgcttccggcgctgaccgaagcatgggccaaaaatccgtctctggatcacctgccggtggtgctcgactggtttaacggccgccgcacaccgaacgctaaccaacgcctgaaaggggtgattaccgatcttaacctcgctaccgacgctccgctgctgttcggcggtttgattgctgccaccgcctttggcgcacgcgcaatcatggagtgctttaccgatcaggggatcgccgttaataacgtgatggcactgggcggcatcgcgcggaaaaaccaggtcattatgcaggcctgctgcgacgtgctgaatcgcccgctgcaaattgttgcctctgaccagtgctgtgcgctcggtgcggcgatttttgctgccgtcgccgcgaaagtgcacgcagacatcccatcagctcagcaaaaaatggccagtgcggtagagaaaaccctgcaaccgtgcagcgagcaggcacaacgctttgaacagctttatcgccgctatcagcaatgggcgatgagcgccgaacaacactatcttccaacttccgccccggcacaggctgcccaggccgttgcgactcta

SEQ ID NO: 24 - protein, 566 aa, Escherichia coli
MAIAIGLDFGSDSVRALAVDCATGEEIATSVEWYPRWQKGQFCDAPNNQFRHHPRDYIESMEAALKTVLAELSVEQRAAVVGIGVDSTGSTPAPIDADGNVLALRPEFAENPNAMFVLWKDHTAVEEAEEITRLCHAPGNVDYSRYIGGIYSSEWFWAKILHVTRQDSAVAQSAASWIELCDWVPALLSGTTRPQDIRRGRCSAGHKSLWHESWGGLPPASFFDELDPILNRHLPSPLFTDTWTADIPVGTLCPEWAQRLGLPESVVISGGAFDCHMGAVGAGAQPNALVKVIGTSTCDILIADKQSVGERAVKGICGQVDGSVVPGFIGLEAGQSAFGDIYAWFGRVLGWPLEQLAAQHPELKTQINASQKQLLPALTEAWAKNPSLDHLPVVLDWFNGRRTPNANQRLKGVITDLNLATDAPLLFGGLIAATAFGARAIMECFTDQGIAVNNVMALGGIARKNQVIMQACCDVLNRPLQIVASDQCCALGAAIFAAVAAKVHADIPSAQQKMASAVEKTLQPCSEQAQRFEQLYRRYQQWAMSAEQHYLPTSAPAQAAQAVATL

SEQ ID NO: 25 - DNA, 693 bp, Escherichia coli
atgttagaagatctcaaacgccaggtattagaagccaacctggcgctgccaaaacacaacctggtcacgctcacatggggcaacgtcagcgccgttgatcgcgagcgcggcgtctttgtgatcaaaccttccggcgtcgattacagcgtcatgaccgctgacgatatggtcgtggttagcatcgaaaccggtgaagtggttgaaggtacgaaaaagccctcctccgacacgccaactcaccggctgctctatcaggcattcccctccattggcggcattgtgcatacgcactcgcgccacgccaccatctgggcgcaggcgggtcagtcgattccagcaaccggcaccacccacgccgactatttctacggcaccattccctgtacccgcaaaatgaccgacgcagaaatcaacggcgaatatgagtgggaaaccggtaacgtcatcgtagaaacctttgaaaaacagggtatcgatgcagcgcaaatgcccggcgttctggtccattcccacggcccgtttgcatggggcaaaaatgccgaagatgcggtgcataacgccatcgtgctggaagaggtcgcttatatggggatattctgccgtcagttagcgccgcagttaccggatatgcagcaaacgctgctggataaacactatctgcgtaagcatggcgcgaaggcatattacgggcag

SEQ ID NO: 26 - protein, 231 aa, Escherichia coli
MLEDLKRQVLEANLALPKHNLVTLTWGNVSAVDRERGVFVIKPSGVDYSVMTADDMVVVSIETGEVVEGTKKPSSDTPTHRLLYQAFPSIGGIVHTHSRHATIWAQAGQSIPATGTTHADYFYGTIPCTRKMTDAEINGEYEWETGNVIVETFEKQGIDAAQMPGVLVHSHGPFAWGKNAEDAVHNAIVLEEVAYMGIFCRQLAPQLPDMQQTLLDKHYLRKHGAKAYYGQ

SEQ ID NO: 27 - DNA, 3226 bp, artificial sequence
  araA-araB PCR fragment
aaccatggcgattgcaattggcctcgattttggcagtgattctgtgcgagctttggcggtggactgcgctaccggtgaagagatcgccaccagcgtagagtggtatccccgttggcagaaagggcaattttgtgatgccccgaataaccagttccgtcatcatccgcgtgactacattgagtcaatggaagcggcactgaaaaccgtgcttgcagagcttagcgtcgaacagcgcgcagctgtggtcgggattggcgttgacagtaccggctcgacgcccgcaccgattgatgccgacggaaacgtgctggcgctgcgcccggagtttgccgaaaacccgaacgcgatgttcgtattgtggaaagaccacactgcggttgaagaagcggaagagattacccgtttgtgccacgcgccgggcaacgttgactactcccgctacattggtggtatttattccagcgaatggttctgggcaaaaatcctgcatgtgactcgccaggacagcgccgtggcgcaatctgccgcatcgtggattgagctgtgcgactgggtgccagctctgctttccggtaccacccgcccgcaggatattcgtcgcggacgttgcagcgccgggcataaatctctgtggcacgaaagctggggcggcctgccgccagccagtttctttgatgagctggacccgatcctcaatcgccatttgccttccccgctgttcactgacacttggactgccgatattccggtgggcaccttatgcccggaatgggcgcagcgtctcggcctgcctgaaagcgtggtgatttccggcggcgcgtttgactgccatatgggcgcagttggcgcaggcgcacagcctaacgcactggtaaaagttatcggtacttccacctgcgacattctgattgccgacaaacagagcgttggcgagcgggcagttaaaggtatttgcggtcaggttgatggcagcgtggtgcctggatttatcggtctggaagcaggccaatcggcgtttggtgatatctacgcctggtttggtcgcgtactcggctggccgctggaacagcttgccgcccagcatccggaactgaaaacgcaaatcaacgccagccagaaacaactgcttccggcgctgaccgaagcatgggccaaaaatccgtctctggatcacctgccggtggtgctcgactggtttaacggccgccgcacaccgaacgctaaccaacgcctgaaaggggtgattaccgatcttaacctcgctaccgacgctccgctgctgttcggcggtttgattgctgccaccgcctttggcgcacgcgcaatcatggagtgctttaccgatcaggggatcgccgttaataacgtgatggcactgggcggcatcgcgcggaaaaaccaggtcattatgcaggcctgctgcgacgtgctgaatcgcccgctgcaaattgttgcctctgaccagtgctgtgcgctcggtgcggcgatttttgctgccgtcgccgcgaaagtgcacgcagacatcccatcagctcagcaaaaaatggccagtgcggtagagaaaaccctgcaaccgtgcagcgagcaggcacaacgctttgaacagctttatcgccgctatcagcaatgggcgatgagcgccgaacaacactatcttccaacttccgccccggcacaggctgcccaggccgttgcgactctataaggacacgataatgacgatttttgataattatgaagtgtggtttgtcattggcagccagcatctgtatggcccggaaaccctgcgtcaggtcacccaacatgccgagcacgtcgttaatgcgctgaatacggaagcgaaactgccctgcaaactggtgttgaaaccgctgggcaccacgccggatgaaatcaccgctatttgccgcgacgcgaattacgacgatcgttgcgctggtctggtggtgtggctgcacaccttctccccggccaaaatgtggatcaacggcctgaccatgctcaacaaaccgttgctgcaattccacacccagttcaacgcggcgctgccgtgggacagtatcgatatggactttatgaacctgaaccagactgcacatggcggtcgcgagttcggcttcattggcgcgcgtatgcgtcagcaacatgccgtggttaccggtcactggcaggataaacaagcccatgagcgtatcggctcctggatgcgtcaggcggtctctaaacaggatacccgtcatctgaaagtctgccgatttggcgataacatgcgtgaagtggcggtcaccgatggcgataaagttgccgcacagatcaagttcggtttctccgtcaatacctgggcggttggcgatctggtgcaggtggtgaactccatcagcgacggcgatgttaacgcgctggtcgatgagtacgaaagctgctacaccatgacgcctgccacacaaatccacggcaaaaaacgacagaacgtgctggaagcggcgcgtattgagctggggatgaagcgtttcctggaacaaggtggcttccacgcgttcaccaccacctttgaagatttgcacggtctgaaacagcttcctggtctggccgtacagcgtctgatgcagcagggttacggctttgcgggcgaaggcgactggaaaactgccgccctgcttcgcatcatgaaggtgatgtcaaccggtctgcagggcggcacctcctttatggaggactacacctatcacttcgagaaaggtaatgacctggtgctcggctcccatatgctggaagtctgcccgtcgatcgccgcagaagagaaaccgatcctcgacgttcagcatctcggtattggtggtaaggacgatcctgcccgcctgatcttcaatacccaaaccggcccagcgattgtcgccagcttgattgatctcggcgatcgttaccgtctactggttaactgcatcgacacggtgaaaacaccgcactccctgccgaaactgccggtggcgaatgcgctgtggaaagcgcaaccggatctgccaactgcttccgaagcgtggatcctcgctggtggcgcgcaccataccgtcttcagccatgcactgaacctcaacgatatgcgccaattcgccgagatgcacgacattgaaatcacggtgattgataacgacacacgcctgccagcgtttaaagacgcgctgcgctggaacgaagtgtattacggatttcgtcgctaagtctagag

SEQ ID NO: 28 - DNA, 25 bp, artificial sequence
  primer
aaccatggcgattgcaattggcctc

SEQ ID NO: 29 - DNA, 32 bp, artificial sequence
  primer
ctctagacttagcgacgaaatccgtaatacac

SEQ ID NO: 30 - DNA, 889 bp, artificial sequence
  araD PCR fragment
gtctagagaaggagtcaacatgttagaagatctcaaacgccaggtattagaagccaacctggcgctgccaaaacacaacctggtcacgctcacatggggcaacgtcagcgccgttgatcgcgagcgcggcgtctttgtgatcaaaccttccggcgtcgattacagcgtcatgaccgctgacgatatggtcgtggttagcatcgaaaccggtgaagtggttgaaggtacgaaaaagccctcctccgacacgccaactcaccggctgctctatcaggcattcccctccattggcggcattgtgcatacgcactcgcgccacgccaccatctgggcgcaggcgggtcagtcgattccagcaaccggcaccacccacgccgactatttctacggcaccattccctgtacccgcaaaatgaccgacgcagaaatcaacggcgaatatgagtgggaaaccggtaacgtcatcgtagaaacctttgaaaaacagggtatcgatgcagcgcaaatgcccggcgttctggtccattcccacggcccgtttgcatggggcaaaaatgccgaagatgcggtgcataacgccatcgtgctggaagaggtcgcttatatggggatattctgccgtcagttagcgccgcagttaccggatatgcagcaaacgctgctggataaacactatctgcgtaagcatggcgcgaaggcatattacgggcagtaatgactgtataaaaccacagccaatcaaacgaaaccaggctatactcaagcctggttttttgatggattttcagcgtggcgcaggcaggttttatcttaacccgacactggcgggacaccccgcaagggacagaagtctccttctggctggcgacggacaacgggccaagcttgg

SEQ ID NO: 31 - DNA, 32 bp, artificial sequence
  primer
gtctagagaaggagtcaacatgttagaagatc

SEQ ID NO: 32 - DNA, 28 bp, artificial sequence
  primer
ccaagcttggcccgttgtccgtcgccag

SEQ ID NO: 33 - DNA, 303 bp, Zymomonas mobilis
tcgatcaacaacccgaatcctatcgtaatgatgttttgcccgatcagcctcaatcgacaattttacgcgtttcgatcgaagcagggacgacaattggctgggaacggtatactggaataaatggtcttcgttatggtattgatgtttttggtgcatcggccccggcgaatgatctatatgctcatttcggcttgaccgcagtcggcatcacgaacaaggtgttggccgcgatcgccggtaagtcggcacgttaaaaaatagctatggaatataatagctacttaataagttaggagaataaac

SEQ ID NO: 34 - DNA, 34 bp, artificial sequence
  primer
gggagctcactagttcgatcaacaacccgaatcc

SEQ ID NO: 35 - DNA, 29 bp, artificial sequence
  primer
agccatggttattctcctaacttattaag

SEQ ID NO: 36 - DNA, 323 bp, artificial sequence
  Pgap PCR fragment
gggagctcactagttcgatcaacaacccgaatcctatcgtaatgatgttttgcccgatcagcctcaatcgacaattttacgcgtttcgatcgaagcagggacgacaattggctgggaacggtatactggaataaatggtcttcgttatggtattgatgtttttggtgcatcggccccggcgaatgatctatatgctcatttcggcttgaccgcagtcggcatcacgaacaaggtgttggccgcgatcgccggtaagtcggcacgttaaaaaatagctatggaatataatagctacttaataagttaggagaataaccatggct

SEQ ID NO: 37 - DNA, 35 bp, artificial sequence
  primer
ctactcatttatcgatggagcacaggatgacgcct

SEQ ID NO: 38 - DNA, 34 bp, artificial sequence
  primer
catcttactacgcgttggcaggtcagcaagtgcc

SEQ ID NO: 39 - DNA, 36 bp, artificial sequence
  mutagenesis oligo
aagttaggagaataaacatggcgattgcaattggcc

SEQ ID NO: 40 - DNA, 36 bp, artificial sequence
  mutagenesis oligo
ggccaattgcaatcgccatgtttattctcctaactt

SEQ ID NO: 41 - DNA, 9884 bp, artificial sequence
  constructed plasmid
ctagttcgatcaacaacccgaatcctatcgtaatgatgttttgcccgatcagcctcaatcgacaattttacgcgtttcgatcgaagcagggacgacaattggctgggaacggtatactggaataaatggtcttcgttatggtattgatgtttttggtgcatcggccccggcgaatgatctatatgctcatttcggcttgaccgcagtcggcatcacgaacaaggtgttggccgcgatcgccggtaagtcggcacgttaaaaaatagctatggaatataatagctacttaataagttaggagaataaacatggcgattgcaattggcctcgattttggcagtgattctgtgcgagctttggcggtggactgcgctaccggtgaagagatcgccaccagcgtagagtggtatccccgttggcagaaagggcaattttgtgatgccccgaataaccagttccgtcatcatccgcgtgactacattgagtcaatggaagcggcactgaaaaccgtgcttgcagagcttagcgtcgaacagcgcgcagctgtggtcgggattggcgttgacagtaccggctcgacgcccgcaccgattgatgccgacggaaacgtgctggcgctgcgcccggagtttgccgaaaacccgaacgcgatgttcgtattgtggaaagaccacactgcggttgaagaagcggaagagattacccgtttgtgccacgcgccgggcaacgttgactactcccgctacattggtggtatttattccagcgaatggttctgggcaaaaatcctgcatgtgactcgccaggacagcgccgtggcgcaatctgccgcatcgtggattgagctgtgcgactgggtgccagctctgctttccggtaccacccgcccgcaggatattcgtcgcggacgttgcagcgccgggcataaatctctgtggcacgaaagctggggcggcctgccgccagccagtttctttgatgagctggacccgatcctcaatcgccatttgccttccccgctgttcactgacacttggactgccgatattccggtgggcaccttatgcccggaatgggcgcagcgtctcggcctgcctgaaagcgtggtgatttccggcggcgcgtttgactgccatatgggcgcagttggcgcaggcgcacagcctaacgcactggtaaaagttatcggtacttccacctgcgacattctgattgccgacaaacagagcgttggcgagcgggcagttaaaggtatttgcggtcaggttgatggcagcgtggtgcctggatttatcggtctggaagcaggccaatcggcgtttggtgatatctacgcctggtttggtcgcgtactcggctggccgctggaacagcttgccgcccagcatccggaactgaaaacgcaaatcaacgccagccagaaacaactgcttccggcgctgaccgaagcatgggccaaaaatccgtctctggatcacctgccggtggtgctcgactggtttaacggccgccgcacaccgaacgctaaccaacgcctgaaaggggtgattaccgatcttaacctcgctaccgacgctccgctgctgttcggcggtttgattgctgccaccgcctttggcgcacgcgcaatcatggagtgctttaccgatcaggggatcgccgttaataacgtgatggcactgggcggcatcgcgcggaaaaaccaggtcattatgcaggcctgctgcgacgtgctgaatcgcccgctgcaaattgttgcctctgaccagtgctgtgcgctcggtgcggcgatttttgctgccgtcgccgcgaaagtgcacgcagacatcccatcagctcagcaaaaaatggccagtgcggtagagaaaaccctgcaaccgtgcagcgagcaggcacaacgctttgaacagctttatcgccgctatcagcaatgggcgatgagcgccgaacaacactatcttccaacttccgccccggcacaggctgcccaggccgttgcgactctataaggacacgataatgacgatttttgataattatgaagtgtggtttgtcattggcagccagcatctgtatggcccggaaaccctgcgtcaggtcacccaacatgccgagcacgtcgttaatgcgctgaatacggaagcgaaactgccctgcaaactggtgttgaaaccgctgggcaccacgccggatgaaatcaccgctatttgccgcgacgcgaattacgacgatcgttgcgctggtctggtggtgtggctgcacaccttctccccggccaaaatgtggatcaacggcctgaccatgctcaacaaaccgttgctgcaattccacacccagttcaacgcggcgctgccgtgggacagtatcgatatggactttatgaacctgaaccagactgcacatggcggtcgcgagttcggcttcattggcgcgcgtatgcgtcagcaacatgccgtggttaccggtcactggcaggataaacaagcccatgagcgtatcggctcctggatgcgtcaggcggtctctaaacaggatacccgtcatctgaaagtctgccgatttggcgataacatgcgtgaagtggcggtcaccgatggcgataaagttgccgcacagatcaagttcggtttctccgtcaatacctgggcggttggcgatctggtgcaggtggtgaactccatcagcgacggcgatgttaacgcgctggtcgatgagtacgaaagctgctacaccatgacgcctgccacacaaatccacggcaaaaaacgacagaacgtgctggaagcggcgcgtattgagctggggatgaagcgtttcctggaacaaggtggcttccacgcgttcaccaccacctttgaagatttgcacggtctgaaacagcttcctggtctggccgtacagcgtctgatgcagcagggttacggctttgcgggcgaaggcgactggaaaactgccgccctgcttcgcatcatgaaggtgatgtcaaccggtctgcagggcggcacctcctttatggaggactacacctatcacttcgagaaaggtaatgacctggtgctcggctcccatatgctggaagtctgcccgtcgatcgccgcagaagagaaaccgatcctcgacgttcagcatctcggtattggtggtaaggacgatcctgcccgcctgatcttcaatacccaaaccggcccagcgattgtcgccagcttgattgatctcggcgatcgttaccgtctactggttaactgcatcgacacggtgaaaacaccgcactccctgccgaaactgccggtggcgaatgcgctgtggaaagcgcaaccggatctgccaactgcttccgaagcgtggatcctcgctggtggcgcgcaccataccgtcttcagccatgcactgaacctcaacgatatgcgccaattcgccgagatgcacgacattgaaatcacggtgattgataacgacacacgcctgccagcgtttaaagacgcgctgcgctggaacgaagtgtattacggatttcgtcgctaagtctagagaaggagtcaacatgttagaagatctcaaacgccaggtattagaagccaacctggcgctgccaaaacacaacctggtcacgctcacatggggcaacgtcagcgccgttgatcgcgagcgcggcgtctttgtgatcaaaccttccggcgtcgattacagcgtcatgaccgctgacgatatggtcgtggttagcatcgaaaccggtgaagtggttgaaggtacgaaaaagccctcctccgacacgccaactcaccggctgctctatcaggcattcccctccattggcggcattgtgcatacgcactcgcgccacgccaccatctgggcgcaggcgggtcagtcgattccagcaaccggcaccacccacgccgactatttctacggcaccattccctgtacccgcaaaatgaccgacgcagaaatcaacggcgaatatgagtgggaaaccggtaacgtcatcgtagaaacctttgaaaaacagggtatcgatgcagcgcaaatgcccggcgttctggtccattcccacggcccgtttgcatggggcaaaaatgccgaagatgcggtgcataacgccatcgtgctggaagaggtcgcttatatggggatattctgccgtcagttagcgccgcagttaccggatatgcagcaaacgctgctggataaacactatctgcgtaagcatggcgcgaaggcatattacgggcagtaatgactgtataaaaccacagccaatcaaacgaaaccaggctatactcaagcctggttttttgatggattttcagcgtggcgcaggcaggttttatcttaacccgacactggcgggacaccccgcaagggacagaagtctccttctggctggcgacggacaacgggccaagcttggaagggcgaattctgcagatatccatcacactggcggccgctaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgtttgacagcttatcatcgatggagcacaggatgacgcctaacaattcattcaagccgacaccgcttcgcggcgcggcttaattcaggagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgactgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaggtagtcggcaaataatgtctaacaattcgttcaagccgacgccgcttcgcggcgcggcttaactcaagcgttagagagctggggaagactatgcgcgatctgttgaaggtggttctaagcctcgtacttgcgatggcatcggggcaggcacttgctgacctgccaacgcgcctttgtagtcttggcctgttgtgtgcatgagcaaatcaatggcaccaccccctcctttttgagctgaatggtcataaaatttataattatctatcgtaattcggaatctatgttcagggtctcgccattgctttttgtctgctgggtcaagttccatgcctaaggtttttaagacatcagaaagaggtattgcacgcatgctatcagcttttcttctagctaatgacagggcttcctctgctctatctgctcgttttttttcttccacatatctcgccgctttgtcagccagcggctgtattacggaaagtgccgatttttgggcttttaggcgttctttttctgcccattcttccttatttgtaaaaattgagggtgggatgggtgcctgaatcttgggatctagctgtaaagttttgttgatatttccgtaatgtctttggactctttgatgcgttgcttttgaaccttttacgcctctggccagccctagaggctccatagaagccgcataatccgtctggagggcagaaagggcttttcgaccatcaaaccatctcgatgcgtttaaacggcctgtatcggggtctctaggcaccataaagccggttaagtggggtgttgtttcatcagcatgtagctgaagagatacaaggttgttttctccaaaggtttgttccgcccattgctgggtgattgttttccagtgttcgagtttttcaggagtggcctgttttgaccattctggagacataccaaagaacagttctatggcctgcacaccgttttttctaagaggctttcccgtttctttctgaattttattcagcatagatttaacatctgctgatgggtcagtagagcctttgagtatttcgtttagttcttttctatctgggtcagcgttttgtgtttcgcggcctcgcgtcatatgcaggctcgcggctttaatcgtgccaactgttttatgtttttcaaacctaaagattgcatagttcggcatgttttaactgctttaatttgagaaaagaccagaggaaataatccagcctatatttctttccctagtagcgaactggaattgtttttccgaaggaaaaaagcaattccgtagtgagtactgaatttattctgattcgtcttgcttttggagcgtctttttgcgttctataactgttgtgaaagctacgcggtcgccattgaaaacgaaattaggattaataaaataccatccttggcgaacatgctttgcaatgattttagctttttctaattcggctagacctcttgcaaaggtagcttgagatagtgccagttttttttcttgtgcgttaagaaagtcctctaaaacgaatttgtctaaagggacgaggtctttgctgatgcctttgtcttgaagtatccaaaccagaacgctgaaagcttttattccagcggctcctagttcaaaagttagcgcgatattggtgctaaataattttacaaattcttcactatcaacacgtctgtaagtcgtcacatgagtgccttgcatctcaccagtggcttgattgaccagaatgttatcatctcgtcctaatcgagataactgaaccctctgacttttaactggcacaaccataccttcgatgaaaggattctcgtcatatctgattggctgctttctcaattttgtcgccatatttgataaacctttaatcaaaaaaaccacattttttgattatacctattcatcgaatgaggcaaggtctatcaattttacccctttttttgatagacggtttaatcaatattgatagaccccttcacagattctgaaaatcgacttccctattttagggatattttcacgattccctttcttagttcttcctagtggggaaattcgttgaatcctgcctcggaaaaaccatgagaaagctgttggttatatacacgggcaaagccaccctatttttagctactggggaaagagataaggcagggtatttgtaaaattaaaaccggatttttcgctttacggtttgtttaggcgcaactgtctttttaagaccgcgtttaaccatcaaaagatcgttccaatcttttccgtgtatcatctgttctttaggtgggagccagttttcaactttttttgttggaaacgcggctttaatcgctccgactaatagcgatgctgctctttgtcctacagcatcccaatcataggcaatatggacagaagatgccttttcaacgatttttcggagagttttagtaagagacgttcttacgccgctggtgcttaataattttacgccagctttaattttttctgggcttaaaaagccgactactgaaatcgcgtctatcgcactttcagcgatataaagatcatacttttcgtcattttttacattgatgctgccagtaaaatgggcttcgcgactgcttcccaaggctaaccctttaaaaccactgcttgttccgcgtaattctgcgccctgaagtgtatctttatcgtcatacatcaagaaggctacattaccgcgatcatctgttcggatagagtcaggaatattgttaaatgatattcctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcccctacgtgctgctgaagttgcccgcaacagagagtggaaccaaccggtgataccacgatactatgactgagagtcaacgccatgggagctca

SEQ ID NO: 42 - DNA, 34 bp, artificial sequence
  primer
atgggagctcgtttttctatccccatcacctcgg

SEQ ID NO: 43 - DNA, 35 bp, artificial sequence
  primer
atcgactagtgggtcataatatgggcaaagacgct

SEQ ID NO: 44 - DNA, 895 bp, artificial sequence
  LDH-L PCR fragment
atgggagctcgtttttctatccccatcacctcggttttgttgacaaaaaaaggtggccactaaattggctttccgcaccgatgggatgatttttattctttgctattcttcgctctttgcccaattcattaaaagcggaaatcatcaccaaagatagaagacgcagccttcaccatttcagattgcccttctcgggcattttctgctgctagaatcctcttaaaaatattaaattccactctattggtaatatgtttccctctttagggaacaaataaagcccttctttgttctataaaagttagcttaccgattttacaaaaaataataccgcttcattcaatcggtaatacatatcttttttcttcaaaaaacttttcaagagggtgtctatgcgcgtcgcaatattcagttccaaaaactatgaccatcattctattgaaaaagaaaatgaacattatggccatgaccttgtttttctgaatgagcggcttaccaaagagacagcagaaaaagccaaagacgcagaagctgtttgtatctttgtgaatgacgaagccaatgccgaagtgctggaaattttggcaggcttaggcatcaagttggttgctcttcgttgcgccggttataacaatgtcgatctcgatgcggccaaaaagctgaatatcaaggttgtgcgcgtgcctgcctattcgccctattcggttgccgaatatgcagtagggatgttgctcaccctgaatcggcaaatttcacgcggtttgaagcgggttcgggaaaataacttctccttggaaggtttgattggccttgatgtgcatgacaaaacagtcggcattatcggtgttggtcatatcgggagcgtctttgcccatattatgacccactagtcgat

SEQ ID NO: 45 - DNA, 33 bp, artificial sequence
  primer
gcgaattcatggttttggtgccaatgttatcgc

SEQ ID NO: 46 - DNA, 35 bp, artificial sequence
  primer
ttaggcggccgcgcggctgacatacatcttgcgaa

SEQ ID NO: 47 - DNA, 1169 bp, artificial sequence
  LDH-R PCR fragment
gcgaattcatggttttggtgccaatgttatcgcctataaaccgcatccagaccccgaattggcgaaaaaggtcggtttccgcttcacctctctcgatgaagtgatcgagaccagcgacatcatttcgcttcactgtccgctcacgccagaaaatcatcacatgattaatgaagaaacactggcaagggcaaaaaaaggcttttacctcgtcaataccagtcgcggcggcttggttgataccaaggcggtgattaaatcgctgaaagccaaacatctcggcggttatgcggcggatgtttacgaagaggaggggcctttattcttcgaaaatcacgctgacgatattatcgaagatgatattctcgaaaggttgatcgctttcccgaatgtggttttcacgggacatcaggcctttttgacgaaagaggccttatcaaacattgctcacagtattctacaagatatcagcgatgccgaagctggaaaagaaatgccggatgcgcttgtttagtagacaagcgacaattaaccttttgaagatcataatgatcaaatttttgggttaattcggtagttatggcataggctattacgcgctaattgatatcaaaaaaaagcatagccggacatcataccggctatgttttttattaggaaaaaatttcctttcaccttgcttagccatcgccgcattatttaatcaatatgccgagtttttcttgaaatccctatcttacaccaaggccaacaagggaatcatccatactcggtgtcctatcctatgactttttaaattttctccaaatttactaaaatcacgccatctcagcggctgctattttcaaaaagcgcctctcaaaaccgctttttcctgctcaaatatcggatcccaaaattccctcaaaaaaggcagggtattttttacaaaatcgcccctaatatctctcaatccgctgccttgttcatatgtttttgcaaatgatttttattaaacttttttaggcgtatttttatcaagaaaatttaaataatcacatttttattattttagatttaagtattgatacaagtgatatctataaatgtttttataactttctggatcgtaatcggctggcaatcgttttccctatattcgcaagatgtatgtcagccgcgcggccgcctaa

SEQ ID NO: 48 - DNA, 1098 bp, artificial sequence
  LoxPw-aadA-LoxPw PCR fragment
ataacttcgtataatgtatgctatacgaagttatgcggccgcagcacaggatgacgcctaacaattcattcaagccgacaccgcttcgcggcgcggcttaattcaggagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgactgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaggtagtcggcaaataatgtctaacaattcgttcaagccgacgccgcttcgcggcgcggcttaactcaagcgttagagagctggggaagactatgcgcgatctgttgaaggtggttctaagcctcgtacttgcgatggcatcggggcaggcacttgctgacctgccttaattaaataacttcgtataatgtatgctatacgaagttat

SEQ ID NO: 49 - DNA, 10441 bp, artificial sequence
  constructed plasmid
ctagttcgatcaacaacccgaatcctatcgtaatgatgttttgcccgatcagcctcaatcgacaattttacgcgtttcgatcgaagcagggacgacaattggctgggaacggtatactggaataaatggtcttcgttatggtattgatgtttttggtgcatcggccccggcgaatgatctatatgctcatttcggcttgaccgcagtcggcatcacgaacaaggtgttggccgcgatcgccggtaagtcggcacgttaaaaaatagctatggaatataatagctacttaataagttaggagaataaacatggcgattgcaattggcctcgattttggcagtgattctgtgcgagctttggcggtggactgcgctaccggtgaagagatcgccaccagcgtagagtggtatccccgttggcagaaagggcaattttgtgatgccccgaataaccagttccgtcatcatccgcgtgactacattgagtcaatggaagcggcactgaaaaccgtgcttgcagagcttagcgtcgaacagcgcgcagctgtggtcgggattggcgttgacagtaccggctcgacgcccgcaccgattgatgccgacggaaacgtgctggcgctgcgcccggagtttgccgaaaacccgaacgcgatgttcgtattgtggaaagaccacactgcggttgaagaagcggaagagattacccgtttgtgccacgcgccgggcaacgttgactactcccgctacattggtggtatttattccagcgaatggttctgggcaaaaatcctgcatgtgactcgccaggacagcgccgtggcgcaatctgccgcatcgtggattgagctgtgcgactgggtgccagctctgctttccggtaccacccgcccgcaggatattcgtcgcggacgttgcagcgccgggcataaatctctgtggcacgaaagctggggcggcctgccgccagccagtttctttgatgagctggacccgatcctcaatcgccatttgccttccccgctgttcactgacacttggactgccgatattccggtgggcaccttatgcccggaatgggcgcagcgtctcggcctgcctgaaagcgtggtgatttccggcggcgcgtttgactgccatatgggcgcagttggcgcaggcgcacagcctaacgcactggtaaaagttatcggtacttccacctgcgacattctgattgccgacaaacagagcgttggcgagcgggcagttaaaggtatttgcggtcaggttgatggcagcgtggtgcctggatttatcggtctggaagcaggccaatcggcgtttggtgatatctacgcctggtttggtcgcgtactcggctggccgctggaacagcttgccgcccagcatccggaactgaaaacgcaaatcaacgccagccagaaacaactgcttccggcgctgaccgaagcatgggccaaaaatccgtctctggatcacctgccggtggtgctcgactggtttaacggccgccgcacaccgaacgctaaccaacgcctgaaaggggtgattaccgatcttaacctcgctaccgacgctccgctgctgttcggcggtttgattgctgccaccgcctttggcgcacgcgcaatcatggagtgctttaccgatcaggggatcgccgttaataacgtgatggcactgggcggcatcgcgcggaaaaaccaggtcattatgcaggcctgctgcgacgtgctgaatcgcccgctgcaaattgttgcctctgaccagtgctgtgcgctcggtgcggcgatttttgctgccgtcgccgcgaaagtgcacgcagacatcccatcagctcagcaaaaaatggccagtgcggtagagaaaaccctgcaaccgtgcagcgagcaggcacaacgctttgaacagctttatcgccgctatcagcaatgggcgatgagcgccgaacaacactatcttccaacttccgccccggcacaggctgcccaggccgttgcgactctataaggacacgataatgacgatttttgataattatgaagtgtggtttgtcattggcagccagcatctgtatggcccggaaaccctgcgtcaggtcacccaacatgccgagcacgtcgttaatgcgctgaatacggaagcgaaactgccctgcaaactggtgttgaaaccgctgggcaccacgccggatgaaatcaccgctatttgccgcgacgcgaattacgacgatcgttgcgctggtctggtggtgtggctgcacaccttctccccggccaaaatgtggatcaacggcctgaccatgctcaacaaaccgttgctgcaattccacacccagttcaacgcggcgctgccgtgggacagtatcgatatggactttatgaacctgaaccagactgcacatggcggtcgcgagttcggcttcattggcgcgcgtatgcgtcagcaacatgccgtggttaccggtcactggcaggataaacaagcccatgagcgtatcggctcctggatgcgtcaggcggtctctaaacaggatacccgtcatctgaaagtctgccgatttggcgataacatgcgtgaagtggcggtcaccgatggcgataaagttgccgcacagatcaagttcggtttctccgtcaatacctgggcggttggcgatctggtgcaggtggtgaactccatcagcgacggcgatgttaacgcgctggtcgatgagtacgaaagctgctacaccatgacgcctgccacacaaatccacggcaaaaaacgacagaacgtgctggaagcggcgcgtattgagctggggatgaagcgtttcctggaacaaggtggcttccacgcgttcaccaccacctttgaagatttgcacggtctgaaacagcttcctggtctggccgtacagcgtctgatgcagcagggttacggctttgcgggcgaaggcgactggaaaactgccgccctgcttcgcatcatgaaggtgatgtcaaccggtctgcagggcggcacctcctttatggaggactacacctatcacttcgagaaaggtaatgacctggtgctcggctcccatatgctggaagtctgcccgtcgatcgccgcagaagagaaaccgatcctcgacgttcagcatctcggtattggtggtaaggacgatcctgcccgcctgatcttcaatacccaaaccggcccagcgattgtcgccagcttgattgatctcggcgatcgttaccgtctactggttaactgcatcgacacggtgaaaacaccgcactccctgccgaaactgccggtggcgaatgcgctgtggaaagcgcaaccggatctgccaactgcttccgaagcgtggatcctcgctggtggcgcgcaccataccgtcttcagccatgcactgaacctcaacgatatgcgccaattcgccgagatgcacgacattgaaatcacggtgattgataacgacacacgcctgccagcgtttaaagacgcgctgcgctggaacgaagtgtattacggatttcgtcgctaagtctagagaaggagtcaacatgttagaagatctcaaacgccaggtattagaagccaacctggcgctgccaaaacacaacctggtcacgctcacatggggcaacgtcagcgccgttgatcgcgagcgcggcgtctttgtgatcaaaccttccggcgtcgattacagcgtcatgaccgctgacgatatggtcgtggttagcatcgaaaccggtgaagtggttgaaggtacgaaaaagccctcctccgacacgccaactcaccggctgctctatcaggcattcccctccattggcggcattgtgcatacgcactcgcgccacgccaccatctgggcgcaggcgggtcagtcgattccagcaaccggcaccacccacgccgactatttctacggcaccattccctgtacccgcaaaatgaccgacgcagaaatcaacggcgaatatgagtgggaaaccggtaacgtcatcgtagaaacctttgaaaaacagggtatcgatgcagcgcaaatgcccggcgttctggtccattcccacggcccgtttgcatggggcaaaaatgccgaagatgcggtgcataacgccatcgtgctggaagaggtcgcttatatggggatattctgccgtcagttagcgccgcagttaccggatatgcagcaaacgctgctggataaacactatctgcgtaagcatggcgcgaaggcatattacgggcagtaatgactgtataaaaccacagccaatcaaacgaaaccaggctatactcaagcctggttttttgatggattttcagcgtggcgcaggcaggttttatcttaacccgacactggcgggacaccccgcaagggacagaagtctccttctggctggcgacggacaacgggccaagcttggaagggcgaattcgcgatcgcataacttcgtataatgtatgctatacgaagttatgcggccgcagcacaggatgacgcctaacaattcattcaagccgacaccgcttcgcggcgcggcttaattcaggagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgactgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaggtagtcggcaaataatgtctaacaattcgttcaagccgacgccgcttcgcggcgcggcttaactcaagcgttagagagctggggaagactatgcgcgatctgttgaaggtggttctaagcctcgtacttgcgatggcatcggggcaggcacttgctgacctgccttaattaaataacttcgtataatgtatgctatacgaagttatggccggccaattcatggttttggtgccaatgttatcgcctataaaccgcatccagaccccgaattggcgaaaaaggtcggtttccgcttcacctctctcgatgaagtgatcgagaccagcgacatcatttcgcttcactgtccgctcacgccagaaaatcatcacatgattaatgaagaaacactggcaagggcaaaaaaaggcttttacctcgtcaataccagtcgcggcggcttggttgataccaaggcggtgattaaatcgctgaaagccaaacatctcggcggttatgcggcggatgtttacgaagaggaggggcctttattcttcgaaaatcacgctgacgatattatcgaagatgatattctcgaaaggttgatcgctttcccgaatgtggttttcacgggacatcaggcctttttgacgaaagaggccttatcaaacattgctcacagtattctacaagatatcagcgatgccgaagctggaaaagaaatgccggatgcgcttgtttagtagacaagcgacaattaaccttttgaagatcataatgatcaaatttttgggttaattcggtagttatggcataggctattacgcgctaattgatatcaaaaaaaagcatagccggacatcataccggctatgttttttattaggaaaaaatttcctttcaccttgcttagccatcgccgcattatttaatcaatatgccgagtttttcttgaaatccctatcttacaccaaggccaacaagggaatcatccatactcggtgtcctatcctatgactttttaaattttctccaaatttactaaaatcacgccatctcagcggctgctattttcaaaaagcgcctctcaaaaccgctttttcctgctcaaatatcggatcccaaaattccctcaaaaaaggcagggtattttttacaaaatcgcccctaatatctctcaatccgctgccttgttcatatgtttttgcaaatgatttttattaaacttttttaggcgtatttttatcaagaaaatttaaataatcacatttttattattttagatttaagtattgatacaagtgatatctataaatgtttttataactttctggatcgtaatcggctggcaatcgttttccctatattcgcaagatgtatgtcagccgcgcggccgctggtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctcgtttttctatccccatcacctcggttttgttgacaaaaaaaggtggccactaaattggctttccgcaccgatgggatgatttttattctttgctattcttcgctctttgcccaattcattaaaagcggaaatcatcaccaaagatagaagacgcagccttcaccatttcagattgcccttctcgggcattttctgctgctagaatcctcttaaaaatattaaattccactctattggtaatatgtttccctctttagggaacaaataaagcccttctttgttctataaaagttagcttaccgattttacaaaaaataataccgcttcattcaatcggtaatacatatcttttttcttcaaaaaacttttcaagagggtgtctatgcgcgtcgcaatattcagttccaaaaactatgaccatcattctattgaaaaagaaaatgaacattatggccatgaccttgtttttctgaatgagcggcttaccaaagagacagcagaaaaagccaaagacgcagaagctgtttgtatctttgtgaatgacgaagccaatgccgaagtgctggaaattttggcaggcttaggcatcaagttggttgctcttcgttgcgccggttataacaatgtcgatctcgatgcggccaaaaagctgaatatcaaggttgtgcgcgtgcctgcctattcgccctattcggttgccgaatatgcagtagggatgttgctcaccctgaatcggcaaatttcacgcggtttgaagcgggttcgggaaaataacttctccttggaaggtttgattggccttgatgtgcatgacaaaacagtcggcattatcggtgttggtcatatcgggagcgtctttgcccatattatgaccca

SEQ ID NO: 50 - DNA, 20 bp, artificial sequence
  primer
gccttgggcttttaaagcct

SEQ ID NO: 51 - DNA, 22 bp, artificial sequence
  primer
tcaatccacgatgcggcagatt

SEQ ID NO: 52 - DNA, 20 bp, artificial sequence
  primer
ccagtatcagcccgtcatac

SEQ ID NO: 53 - DNA, 26 bp, artificial sequence
  primer
tctcggagagatagaggtcagtcgac

SEQ ID NO: 54 - DNA, 27 bp, artificial sequence
  primer
aaccatggttactatcaatacggaatc

SEQ ID NO: 55 - DNA, 27 bp, artificial sequence
  primer
ttgaattcctgatgtgtgttaccgcaa

SEQ ID NO: 56 - DNA, 1550 bp, artificial sequence
  araE PCR fragment
aaccatggttactatcaatacggaatctgctttaacgccacgttctttgcgggatacgcggcgtatgaatatgtttgtttcggtagctgctgcggtcgcaggattgttatttggtcttgatatcggcgtaatcgccggagcgttgccgttcattaccgatcactttgtgctgaccagtcgtttgcaggaatgggtggttagtagcatgatgctcggtgcagcaattggtgcgctgtttaatggttggctgtcgttccgcctggggcgtaaatacagcctgatggcgggggccatcctgtttgtactcggttctatagggtccgcttttgcgaccagcgtagagatgttaatcgccgctcgtgtggtgctgggcattgctgtcgggatcgcgtcttacaccgctcctctgtatctttctgaaatggcaagtgaaaacgttcgcggtaagatgatcagtatgtaccagttgatggtcacactcggcatcgtgctggcgtttttatccgatacagcgttcagttatagcggtaactggcgcgcaatgttgggggttcttgctttaccagcagttctgctgattattctggtagtcttcctgccaaatagcccgcgctggctggcggaaaaggggcgtcatattgaggcggaagaagtattgcgtatgctgcgcgatacgtcggaaaaagcgcgagaagaactcaacgaaattcgtgaaagcctgaagttaaaacagggcggttgggcactgtttaagatcaaccgtaacgtccgtcgtgctgtgtttctcggtatgttgttgcaggcgatgcagcagtttaccggtatgaacatcatcatgtactacgcgccgcgtatcttcaaaatggcgggctttacgaccacagaacaacagatgattgcgactctggtcgtagggctgacctttatgttcgccacctttattgcggtgtttacggtagataaagcagggcgtaaaccggctctgaaaattggtttcagcgtgatggcgttaggcactctggtgctgggctattgcctgatgcagtttgataacggtacggcttccagtggcttgtcctggctctctgttggcatgacgatgatgtgtattgccggttatgcgatgagcgccgcgccagtggtgtggatcctgtgctctgaaattcagccgctgaaatgccgcgatttcggtattacctgttcgaccaccacgaactgggtgtcgaatatgattatcggcgcgaccttcctgacactgcttgatagcattggcgctgccggtacgttctggctctacactgcgctgaacattgcgtttgtgggcattactttctggctcattccggaaaccaaaaatgtcacgctggaacatatcgaacgcaaactgatggcaggcgagaagttgagaaatatcggcgtctgatttcacgggccggatgtgctgtacatccggcccttttttcgttaatagagattgggcacttggccgttgaggcgtttgtctcgttccttattcagccttgttgcggtaacacacatcaggaattcaa

SEQ ID NO: 57 - DNA, 32 bp, artificial sequence
  primer
aaccatggcgcacaaatttactaaagccctgg

SEQ ID NO: 58 - DNA, 30 bp, artificial sequence
  primer
ccgaattccttctcttttcttattgtgttg

SEQ ID NO: 59 - DNA, 3744 bp, artificial sequence
  araFGH PCR fragment
aaccatggcgcacaaatttactaaagccctggcagccattggtctggcagccgttatgtcacaatccgctatggcggagaacctgaagctcggttttctggtgaagcaaccggaagagccgtggttccagaccgaatggaagtttgccgataaagccgggaaggatttagggtttgaggttattaagattgccgtgccggatggcgaaaaaacattgaacgcgatcgacagcctggctgccagtggcgcaaaaggtttcgttatttgtactccggaccccaaactcggctctgccatcgtcgcgaaagcgcgtggctacgatatgaaagtcattgccgtggatgaccagtttgttaacgccaaaggtaagccaatggataccgttccgctggtgatgatggcggcgactaaaattggcgaacgtcagggccaggaactgtataaagagatgcagaaacgtggctgggatgtcaaagaaagcgcggtgatggcgattaccgccaacgaactggataccgcccgccgccgtactacgggatctatggatgcgctgaaagcggccggattcccggaaaaacaaatttatcaggtacctaccaaatctaacgacatcccgggggcatttgacgctgccaactcaatgctggttcaacatccggaagttaaacattggctgatcgtcggtatgaacgacagcaccgtgctgggcggcgtacgcgcgacggaaggtcagggctttaaagcggccgatatcatcggcattggcattaacggtgtggatgcggtgagcgaactgtctaaagcacaggcaaccggcttctacggttccctgctgccaagcccggacgtacatggctataaatccagcgaaatgctttacaactgggtagcaaaagacgttgaaccgccaaaatttaccgaagttaccgacgtggtactgatcacgcgtgacaactttaaagaagaactggagaaaaaaggtttaggcggtaagtaatttgccggaaaaattcccctctgcatgatgcagagggggtgtgaacgaccagtgattcacggagacgttatgcaacagtctaccccgtatctctcatttcgcggcatcggtaaaacgtttcccggcgttaaggcgctgacggatattagttttgactgctatgccggtcaggttcatgcgttgatgggtgaaaatggcgcaggaaaatcaactctcttaaaaatcctcagcggcaactatgcgccaaccacgggttctgtagtgattaatgggcaggaaatgtccttttccgacacgaccgcagcacttaacgcgggcgtggcgattatttaccaggaactgcatctcgtgccggaaatgaccgtcgcggaaaacatctatctcggccagctgccgcataaaggcggcattgtgaatcgctcattgctgaattatgaggcgggtttacaacttaaacatcttggtatggatattgacccggacacgccgctgaaatatctctccattggtcagtggcagatggttgaaatcgccaaagcgctggcgcgtaacgccaaaattatcgcctttgatgagccaaccagctccctctctgcccgtgaaatcgacaatcttttccgcgttattcgtgaactgcgaaaagaggggcgggtaatcttatacgtttctcaccgtatggaagaaatatttgccctcagcgatgccattactgtctttaaagatggacgttatgtcaaaacctttaccgatatgcagcaggttgaccacgacgcgctggtgcaggcgatggtcgggcgcgacattggcgatatctacggctggcaaccgcgtagttatggcgaggagcgcctacgtcttgatgctgtgaaagcaccaggcgtgcgtacgccaataagtctggcggttcgcagtggtgaaattgttgggctgtttggtctggtaggggcggggcgtagcgaattaatgaaaggcatgtttggcgggacgcaaatcaccgccggtcaggtttatatcgaccaacagccgatcgatattcgtaaaccgagccacgccattgccgcaggcatgatgctctgcccggaagatcgcaaagcggaaggcattattcccgtgcactccgttcgcgacaatatcaacatcagtgccagacgtaaacatgtgctcggcggttgtgtaatcaacaacggttgggaagaaaacaatgccgatcaccacattcgttcgctcaacatcaaaacgccgggcgcggagcaactgatcatgaatctctcaggcggaaatcagcaaaaagccattctgggccgctggttatcggaagagatgaaggtcattttgctggatgaacctacgcgcggcattgatgttggcgctaagcacgaaatatataacgtaatttatgcgctggcggcgcagggcgtggcggtgctgtttgcctccagcgacttacctgaagtcctcggcgttgccgaccggattgtggtgatgcgggaaggtgaaatcgccggtgaattgttacacgagcaggcagatgagcgtcaggcactgagccttgcgatgcctaaagtcagccaggctgttgcctgagtaaggagagtatgatgtcttctgtttctacatcggggtctggcgcacctaagtcgtcattcagcttcgggcgtatctgggatcagtacggcatgctggtggtgtttgcggtgctctttatcgcctgtgccatttttgtcccaaattttgccaccttcattaatatgaaagggttgggcctggcaatttccatgtcggggatggtggcttgtggcatgttgttctgcctcgcttccggtgactttgacctttctgtcgcctccgtaattgcctgtgcgggtgtcaccacggcggtggttattaacctgactgaaagcctgtggattggcgtggcagcggggttgttgctgggcgttctctgtggcctggtcaatggctttgttatcgccaaactgaaaataaatgctctgatcacgacattggcaacgatgcagattgttcgaggtctggcgtacatcatttcagacggtaaagcggtcggtatcgaagatgaaagcttctttgcccttggttacgccaactggttcggtctgcctgcgccaatctggctcaccgtcgcgtgtctgattatctttggtttgctgctgaataaaaccacctttggtcgtaacaccctggcgattggcgggaacgaagaggccgcgcgtctggcgggtgtaccggttgttcgcaccaaaattattatctttgttctctcaggcctggtatcagcgatagccggaattattctggcttcacgtatgaccagtgggcagccaatgacgtcgattggttatgagctgattgttatctccgcctgcgttttaggtggcgtttctctgaaaggtggcatcggaaaaatctcatatgtggtggcgggtatcttaattttaggcaccgtggaaaacgccatgaacctgcttaatatttctcctttcgcgcagtacgtggttcgcggcttaatcctgctggcagcggtgatcttcgaccgttacaagcaaaaagcgaaacgcactgtctgatgcttttttctgcaacaatttagcgttttttcccaccatagccaaccgccataacggttggctgttcttcgttgcaaatggcgacccccgtcacactgtctatacttacatgtctgtaaagcgcgttctgcgcaacacaataagaaaagagaaggaattcgg

SEQ ID NO: 60 - DNA, 27 bp, artificial sequence
  primer
gggagctcactagtcgatctgtgctgt

SEQ ID NO: 61 - DNA, 23 bp, artificial sequence
  primer
agccatggttacctccgggaaac

SEQ ID NO: 62 - DNA, 181 bp, Actinoplanes missouriensis
cgatctgtgctgtttgccacggtatgcagcaccagcgcgagattatgggctcgcacgctcgactgtcggacgggggcactggaacgagaagtcaggcgagccgtcacgcccttgacaatgccacatcctgagcaaataattcaaccactaaacaaatcaaccgcgtttcccggaggtaacc

SEQ ID NO: 63 - DNA, 201 bp, artificial sequence
  Pgi PCR fragment
gggagctcactagtcgatctgtgctgtttgccacggtatgcagcaccagcgcgagattatgggctcgcacgctcgactgtcggacgggggcactggaacgagaagtcaggcgagccgtcacgcccttgacaatgccacatcctgagcaaataattcaaccactaaacaaatcaaccgcgtttcccggaggtaaccatggct

SEQ ID NO: 64 - DNA, 911 bp, artificial sequence
  chloramphenicol resistance marker
gtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggttgctacgcctgaataag

SEQ ID NO: 65 - DNA, 7224 bp, artificial sequence
  constructed plasmid
ggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgtttgacagcttatcatcgatggagcacaggatgacgcctaacaattcattcaagccgacaccgcttcgcggcgcggcttaattcaggagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgactgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaggtagtcggcaaataatgtctaacaattcgttcaagccgacgccgcttcgcggcgcggcttaactcaagcgttagagagctggggaagactatgcgcgatctgttgaaggtggttctaagcctcgtacttgcgatggcatcggggcaggcacttgctgacctgccaacgcgcctttgtagtcttggcctgttgtgtgcatgagcaaatcaatggcaccaccccctcctttttgagctgaatggtcataaaatttataattatctatcgtaattcggaatctatgttcagggtctcgccattgctttttgtctgctgggtcaagttccatgcctaaggtttttaagacatcagaaagaggtattgcacgcatgctatcagcttttcttctagctaatgacagggcttcctctgctctatctgctcgttttttttcttccacatatctcgccgctttgtcagccagcggctgtattacggaaagtgccgatttttgggcttttaggcgttctttttctgcccattcttccttatttgtaaaaattgagggtgggatgggtgcctgaatcttgggatctagctgtaaagttttgttgatatttccgtaatgtctttggactctttgatgcgttgcttttgaaccttttacgcctctggccagccctagaggctccatagaagccgcataatccgtctggagggcagaaagggcttttcgaccatcaaaccatctcgatgcgtttaaacggcctgtatcggggtctctaggcaccataaagccggttaagtggggtgttgtttcatcagcatgtagctgaagagatacaaggttgttttctccaaaggtttgttccgcccattgctgggtgattgttttccagtgttcgagtttttcaggagtggcctgttttgaccattctggagacataccaaagaacagttctatggcctgcacaccgttttttctaagaggctttcccgtttctttctgaattttattcagcatagatttaacatctgctgatgggtcagtagagcctttgagtatttcgtttagttcttttctatctgggtcagcgttttgtgtttcgcggcctcgcgtcatatgcaggctcgcggctttaatcgtgccaactgttttatgtttttcaaacctaaagattgcatagttcggcatgttttaactgctttaatttgagaaaagaccagaggaaataatccagcctatatttctttccctagtagcgaactggaattgtttttccgaaggaaaaaagcaattccgtagtgagtactgaatttattctgattcgtcttgcttttggagcgtctttttgcgttctataactgttgtgaaagctacgcggtcgccattgaaaacgaaattaggattaataaaataccatccttggcgaacatgctttgcaatgattttagctttttctaattcggctagacctcttgcaaaggtagcttgagatagtgccagttttttttcttgtgcgttaagaaagtcctctaaaacgaatttgtctaaagggacgaggtctttgctgatgcctttgtcttgaagtatccaaaccagaacgctgaaagcttttattccagcggctcctagttcaaaagttagcgcgatattggtgctaaataattttacaaattcttcactatcaacacgtctgtaagtcgtcacatgagtgccttgcatctcaccagtggcttgattgaccagaatgttatcatctcgtcctaatcgagataactgaaccctctgacttttaactggcacaaccataccttcgatgaaaggattctcgtcatatctgattggctgctttctcaattttgtcgccatatttgataaacctttaatcaaaaaaaccacattttttgattatacctattcatcgaatgaggcaaggtctatcaattttacccctttttttgatagacggtttaatcaatattgatagaccccttcacagattctgaaaatcgacttccctattttagggatattttcacgattccctttcttagttcttcctagtggggaaattcgttgaatcctgcctcggaaaaaccatgagaaagctgttggttatatacacgggcaaagccaccctatttttagctactggggaaagagataaggcagggtatttgtaaaattaaaaccggatttttcgctttacggtttgtttaggcgcaactgtctttttaagaccgcgtttaaccatcaaaagatcgttccaatcttttccgtgtatcatctgttctttaggtgggagccagttttcaactttttttgttggaaacgcggctttaatcgctccgactaatagcgatgctgctctttgtcctacagcatcccaatcataggcaatatggacagaagatgccttttcaacgatttttcggagagttttagtaagagacgttcttacgccgctggtgcttaataattttacgccagctttaattttttctgggcttaaaaagccgactactgaaatcgcgtctatcgcactttcagcgatataaagatcatacttttcgtcattttttacattgatgctgccagtaaaatgggcttcgcgactgcttcccaaggctaaccctttaaaaccactgcttgttccgcgtaattctgcgccctgaagtgtatctttatcgtcatacatcaagaaggctacattaccgcgatcatctgttcggatagagtcaggaatattgttaaatgatattcctcggctagtcgatctgtgctgtttgccacggtatgcagcaccagcgcgagattatgggctcgcacgctcgactgtcggacgggggcactggaacgagaagtcaggcgagccgtcacgcccttgacaatgccacatcctgagcaaataattcaaccactaaacaaatcaaccgcgtttcccggaggtaaccatggttactatcaatacggaatctgctttaacgccacgttctttgcgggatacgcggcgtatgaatatgtttgtttcggtagctgctgcggtcgcaggattgttatttggtcttgatatcggcgtaatcgccggagcgttgccgttcattaccgatcactttgtgctgaccagtcgtttgcaggaatgggtggttagtagcatgatgctcggtgcagcaattggtgcgctgtttaatggttggctgtcgttccgcctggggcgtaaatacagcctgatggcgggggccatcctgtttgtactcggttctatagggtccgcttttgcgaccagcgtagagatgttaatcgccgctcgtgtggtgctgggcattgctgtcgggatcgcgtcttacaccgctcctctgtatctttctgaaatggcaagtgaaaacgttcgcggtaagatgatcagtatgtaccagttgatggtcacactcggcatcgtgctggcgtttttatccgatacagcgttcagttatagcggtaactggcgcgcaatgttgggggttcttgctttaccagcagttctgctgattattctggtagtcttcctgccaaatagcccgcgctggctggcggaaaaggggcgtcatattgaggcggaagaagtattgcgtatgctgcgcgatacgtcggaaaaagcgcgagaagaactcaacgaaattcgtgaaagcctgaagttaaaacagggcggttgggcactgtttaagatcaaccgtaacgtccgtcgtgctgtgtttctcggtatgttgttgcaggcgatgcagcagtttaccggtatgaacatcatcatgtactacgcgccgcgtatcttcaaaatggcgggctttacgaccacagaacaacagatgattgcgactctggtcgtagggctgacctttatgttcgccacctttattgcggtgtttacggtagataaagcagggcgtaaaccggctctgaaaattggtttcagcgtgatggcgttaggcactctggtgctgggctattgcctgatgcagtttgataacggtacggcttccagtggcttgtcctggctctctgttggcatgacgatgatgtgtattgccggttatgcgatgagcgccgcgccagtggtgtggatcctgtgctctgaaattcagccgctgaaatgccgcgatttcggtattacctgttcgaccaccacgaactgggtgtcgaatatgattatcggcgcgaccttcctgacactgcttgatagcattggcgctgccggtacgttctggctctacactgcgctgaacattgcgtttgtgggcattactttctggctcattccggaaaccaaaaatgtcacgctggaacatatcgaacgcaaactgatggcaggcgagaagttgagaaatatcggcgtctgatttcacgggccggatgtgctgtacatccggcccttttttcgttaatagagattgggcacttggccgttgaggcgtttgtctcgttccttattcagccttgttgcggtaacacacatcaggaattctgcagatatccatcacactggcggccgcgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggttgctacgcctgaataagttaattaatgcgctagcggagtgtatact

SEQ ID NO: 66 - DNA, 9418 bp, artificial sequence
  constructed plasmid
ctagtcgatctgtgctgtttgccacggtatgcagcaccagcgcgagattatgggctcgcacgctcgactgtcggacgggggcactggaacgagaagtcaggcgagccgtcacgcccttgacaatgccacatcctgagcaaataattcaaccactaaacaaatcaaccgcgtttcccggaggtaaccatggcgcacaaatttactaaagccctggcagccattggtctggcagccgttatgtcacaatccgctatggcggagaacctgaagctcggttttctggtgaagcaaccggaagagccgtggttccagaccgaatggaagtttgccgataaagccgggaaggatttagggtttgaggttattaagattgccgtgccggatggcgaaaaaacattgaacgcgatcgacagcctggctgccagtggcgcaaaaggtttcgttatttgtactccggaccccaaactcggctctgccatcgtcgcgaaagcgcgtggctacgatatgaaagtcattgccgtggatgaccagtttgttaacgccaaaggtaagccaatggataccgttccgctggtgatgatggcggcgactaaaattggcgaacgtcagggccaggaactgtataaagagatgcagaaacgtggctgggatgtcaaagaaagcgcggtgatggcgattaccgccaacgaactggataccgcccgccgccgtactacgggatctatggatgcgctgaaagcggccggattcccggaaaaacaaatttatcaggtacctaccaaatctaacgacatcccgggggcatttgacgctgccaactcaatgctggttcaacatccggaagttaaacattggctgatcgtcggtatgaacgacagcaccgtgctgggcggcgtacgcgcgacggaaggtcagggctttaaagcggccgatatcatcggcattggcattaacggtgtggatgcggtgagcgaactgtctaaagcacaggcaaccggcttctacggttccctgctgccaagcccggacgtacatggctataaatccagcgaaatgctttacaactgggtagcaaaagacgttgaaccgccaaaatttaccgaagttaccgacgtggtactgatcacgcgtgacaactttaaagaagaactggagaaaaaaggtttaggcggtaagtaatttgccggaaaaattcccctctgcatgatgcagagggggtgtgaacgaccagtgattcacggagacgttatgcaacagtctaccccgtatctctcatttcgcggcatcggtaaaacgtttcccggcgttaaggcgctgacggatattagttttgactgctatgccggtcaggttcatgcgttgatgggtgaaaatggcgcaggaaaatcaactctcttaaaaatcctcagcggcaactatgcgccaaccacgggttctgtagtgattaatgggcaggaaatgtccttttccgacacgaccgcagcacttaacgcgggcgtggcgattatttaccaggaactgcatctcgtgccggaaatgaccgtcgcggaaaacatctatctcggccagctgccgcataaaggcggcattgtgaatcgctcattgctgaattatgaggcgggtttacaacttaaacatcttggtatggatattgacccggacacgccgctgaaatatctctccattggtcagtggcagatggttgaaatcgccaaagcgctggcgcgtaacgccaaaattatcgcctttgatgagccaaccagctccctctctgcccgtgaaatcgacaatcttttccgcgttattcgtgaactgcgaaaagaggggcgggtaatcttatacgtttctcaccgtatggaagaaatatttgccctcagcgatgccattactgtctttaaagatggacgttatgtcaaaacctttaccgatatgcagcaggttgaccacgacgcgctggtgcaggcgatggtcgggcgcgacattggcgatatctacggctggcaaccgcgtagttatggcgaggagcgcctacgtcttgatgctgtgaaagcaccaggcgtgcgtacgccaataagtctggcggttcgcagtggtgaaattgttgggctgtttggtctggtaggggcggggcgtagcgaattaatgaaaggcatgtttggcgggacgcaaatcaccgccggtcaggtttatatcgaccaacagccgatcgatattcgtaaaccgagccacgccattgccgcaggcatgatgctctgcccggaagatcgcaaagcggaaggcattattcccgtgcactccgttcgcgacaatatcaacatcagtgccagacgtaaacatgtgctcggcggttgtgtaatcaacaacggttgggaagaaaacaatgccgatcaccacattcgttcgctcaacatcaaaacgccgggcgcggagcaactgatcatgaatctctcaggcggaaatcagcaaaaagccattctgggccgctggttatcggaagagatgaaggtcattttgctggatgaacctacgcgcggcattgatgttggcgctaagcacgaaatatataacgtaatttatgcgctggcggcgcagggcgtggcggtgctgtttgcctccagcgacttacctgaagtcctcggcgttgccgaccggattgtggtgatgcgggaaggtgaaatcgccggtgaattgttacacgagcaggcagatgagcgtcaggcactgagccttgcgatgcctaaagtcagccaggctgttgcctgagtaaggagagtatgatgtcttctgtttctacatcggggtctggcgcacctaagtcgtcattcagcttcgggcgtatctgggatcagtacggcatgctggtggtgtttgcggtgctctttatcgcctgtgccatttttgtcccaaattttgccaccttcattaatatgaaagggttgggcctggcaatttccatgtcggggatggtggcttgtggcatgttgttctgcctcgcttccggtgactttgacctttctgtcgcctccgtaattgcctgtgcgggtgtcaccacggcggtggttattaacctgactgaaagcctgtggattggcgtggcagcggggttgttgctgggcgttctctgtggcctggtcaatggctttgttatcgccaaactgaaaataaatgctctgatcacgacattggcaacgatgcagattgttcgaggtctggcgtacatcatttcagacggtaaagcggtcggtatcgaagatgaaagcttctttgcccttggttacgccaactggttcggtctgcctgcgccaatctggctcaccgtcgcgtgtctgattatctttggtttgctgctgaataaaaccacctttggtcgtaacaccctggcgattggcgggaacgaagaggccgcgcgtctggcgggtgtaccggttgttcgcaccaaaattattatctttgttctctcaggcctggtatcagcgatagccggaattattctggcttcacgtatgaccagtgggcagccaatgacgtcgattggttatgagctgattgttatctccgcctgcgttttaggtggcgtttctctgaaaggtggcatcggaaaaatctcatatgtggtggcgggtatcttaattttaggcaccgtggaaaacgccatgaacctgcttaatatttctcctttcgcgcagtacgtggttcgcggcttaatcctgctggcagcggtgatcttcgaccgttacaagcaaaaagcgaaacgcactgtctgatgcttttttctgcaacaatttagcgttttttcccaccatagccaaccgccataacggttggctgttcttcgttgcaaatggcgacccccgtcacactgtctatacttacatgtctgtaaagcgcgttctgcgcaacacaataagaaaagagaaggaattctgcagatatccatcacactggcggccgcgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggttgctacgcctgaataagttaattaatgcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgtttgacagcttatcatcgatggagcacaggatgacgcctaacaattcattcaagccgacaccgcttcgcggcgcggcttaattcaggagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgactgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaggtagtcggcaaataatgtctaacaattcgttcaagccgacgccgcttcgcggcgcggcttaactcaagcgttagagagctggggaagactatgcgcgatctgttgaaggtggttctaagcctcgtacttgcgatggcatcggggcaggcacttgctgacctgccaacgcgcctttgtagtcttggcctgttgtgtgcatgagcaaatcaatggcaccaccccctcctttttgagctgaatggtcataaaatttataattatctatcgtaattcggaatctatgttcagggtctcgccattgctttttgtctgctgggtcaagttccatgcctaaggtttttaagacatcagaaagaggtattgcacgcatgctatcagcttttcttctagctaatgacagggcttcctctgctctatctgctcgttttttttcttccacatatctcgccgctttgtcagccagcggctgtattacggaaagtgccgatttttgggcttttaggcgttctttttctgcccattcttccttatttgtaaaaattgagggtgggatgggtgcctgaatcttgggatctagctgtaaagttttgttgatatttccgtaatgtctttggactctttgatgcgttgcttttgaaccttttacgcctctggccagccctagaggctccatagaagccgcataatccgtctggagggcagaaagggcttttcgaccatcaaaccatctcgatgcgtttaaacggcctgtatcggggtctctaggcaccataaagccggttaagtggggtgttgtttcatcagcatgtagctgaagagatacaaggttgttttctccaaaggtttgttccgcccattgctgggtgattgttttccagtgttcgagtttttcaggagtggcctgttttgaccattctggagacataccaaagaacagttctatggcctgcacaccgttttttctaagaggctttcccgtttctttctgaattttattcagcatagatttaacatctgctgatgggtcagtagagcctttgagtatttcgtttagttcttttctatctgggtcagcgttttgtgtttcgcggcctcgcgtcatatgcaggctcgcggctttaatcgtgccaactgttttatgtttttcaaacctaaagattgcatagttcggcatgttttaactgctttaatttgagaaaagaccagaggaaataatccagcctatatttctttccctagtagcgaactggaattgtttttccgaaggaaaaaagcaattccgtagtgagtactgaatttattctgattcgtcttgcttttggagcgtctttttgcgttctataactgttgtgaaagctacgcggtcgccattgaaaacgaaattaggattaataaaataccatccttggcgaacatgctttgcaatgattttagctttttctaattcggctagacctcttgcaaaggtagcttgagatagtgccagttttttttcttgtgcgttaagaaagtcctctaaaacgaatttgtctaaagggacgaggtctttgctgatgcctttgtcttgaagtatccaaaccagaacgctgaaagcttttattccagcggctcctagttcaaaagttagcgcgatattggtgctaaataattttacaaattcttcactatcaacacgtctgtaagtcgtcacatgagtgccttgcatctcaccagtggcttgattgaccagaatgttatcatctcgtcctaatcgagataactgaaccctctgacttttaactggcacaaccataccttcgatgaaaggattctcgtcatatctgattggctgctttctcaattttgtcgccatatttgataaacctttaatcaaaaaaaccacattttttgattatacctattcatcgaatgaggcaaggtctatcaattttacccctttttttgatagacggtttaatcaatattgatagaccccttcacagattctgaaaatcgacttccctattttagggatattttcacgattccctttcttagttcttcctagtggggaaattcgttgaatcctgcctcggaaaaaccatgagaaagctgttggttatatacacgggcaaagccaccctatttttagctactggggaaagagataaggcagggtatttgtaaaattaaaaccggatttttcgctttacggtttgtttaggcgcaactgtctttttaagaccgcgtttaaccatcaaaagatcgttccaatcttttccgtgtatcatctgttctttaggtgggagccagttttcaactttttttgttggaaacgcggctttaatcgctccgactaatagcgatgctgctctttgtcctacagcatcccaatcataggcaatatggacagaagatgccttttcaacgatttttcggagagttttagtaagagacgttcttacgccgctggtgcttaataattttacgccagctttaattttttctgggcttaaaaagccgactactgaaatcgcgtctatcgcactttcagcgatataaagatcatacttttcgtcattttttacattgatgctgccagtaaaatgggcttcgcgactgcttcccaaggctaaccctttaaaaccactgcttgttccgcgtaattctgcgccctgaagtgtatctttatcgtcatacatcaagaaggctacattaccgcgatcatctgttcggatagagtcaggaatattgttaaatgatattcctcgg